SEQUENCE LISTING (3 sequences)

SEQ ID NO: 1 - protein, 162 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 2 - protein, 253 aa, Homo sapiens
MWPPGSASQPPPSPAAATGLHPAARPVSLQCRLSMCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 3 - protein, 328 aa, Homo sapiens
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

(19) United States
(12) Patent Application Publication
(10) Pub. No.: US 2024/0082398 A1
(43) Pub. Date: Mar. 14, 2024

(54) METHODS OF PREPARING AND EXPANDING TYPE I INNATE LYMPHOID CELLS AND THERAPEUTIC USES THEREOF

(71) Applicant: City of Hope, Duart, CA (US)

(72) Inventors: Jianhua Yu, Duarte, CA (US); Michael A. Caligiuri, Pasadena, CA (US)

(21) Appl. No.: 18/272,403

(22) PCT Filed: Jan. 18, 2022
(86) PCT No.: PCT/US2022/012731

§ 371 Date: Jul. 14, 2023

Related U.S. Application Data
(60) Provisional application No. 63/138,376, filed on Jan. 15, 2021.

(51) Int. Cl. A61K 39/00; A61P 35/02; C07K 16/28; C12N 5/078

(52) U.S. Cl. CPC A61K 39/4611; A61K 39/4622; A61K 39/4637; A61K 39/4643; A61K 39/464411; A61K 39/464419; A61K 39/464429; A61K 39/46444; A61P 35/02; C07K 16/2827; C12N 5/0634; A61K 2239/48; C12N 2501/2302; C12N 2501/2307; C12N 2502/30

(57) ABSTRACT

Provided herein are, inter alia, compositions comprising ex vivo expanded ILC1 cells, methods of preparing the compositions, and methods useful for treating cancer and leukemia.

### CLAIM OF PRIORITY

This application claims the benefit of U.S. Provisional Application Ser. No. 63/138,376, filed on Jan. 15, 2021. The entire contents of the foregoing are incorporated herein by reference.

### BACKGROUND

Acute myeloid leukemia (AML) usually occurs following the successive acquisition of mutations in hematopoietic stem and progenitor cells. AML is a highly heterogeneous and aggressive malignancy characterized by morphologic and chromosomal aberrations with high mortality despite treatment. With the exception of those undergoing hematopoietic stem cell transplantation (HSCT), only a small fraction of patients are cured of the disease¹. However, older adult patients are often ineligible for HSCT due to co-morbid conditions². Due to the persistence of leukemia stem cells (LSCs) following standard chemotherapy, relapse is all but certain and highly fatal; 5-year overall survival is only 40-45% in pediatric and younger adult patients (<40 years of age) and 5-15% in older AML patients (>60 years of age)³⁻⁵. How immune cells interact with leukemia stem cells (LSCs) to prevent AML relapse is largely unknown. Therefore, identifying and exploiting the underlying mechanisms to prevent AML relapse is of significance and is an unmet medical need.

### SUMMARY

Type I innate lymphoid cells (ILC1s) play a critical role in regulating inflammation and immunity in mammalian tissues. However, their functional roles in cancer immunity and immunotherapy are less defined. This application is based in part on the surprising discovery that isolated ILC1s induce leukemia stem cell (LSC; Lin⁻Sca-1⁺c-Kit⁺) apoptosis, promote LSC differentiation into Lin⁻Sca-1⁺c-Kit⁻non-leukemic cells, suppress LSC differentiation into Lin⁻Sca-1⁻c-Kit⁺leukemia progenitor cells, and thereby block differentiation into terminal myeloid blasts. Without being bound by theory, ILC1s produce abundant interferon-γ (IFN-γ), particularly when stimulated by tumor cells, and ultimately suppress leukemogenesis. Also without being bound by theory, inhibition of JAK-STAT and PI3K-AKT signaling pathways in LSCs decrease the anti-leukemic effects of ILC1s. As described herein, inter alia, ILC act as anti-cancer immune cells suitable for immunotherapy. In some aspects, the ILC1s are used to treat a cancer or leukemia (e.g., acute lymphocytic leukemia (ALL), acute myeloid leukemia (AML), chronic lymphocytic leukemia (CLL), chronic myeloid leukemia (CML), hairy cell leukemia (HCL), or myelodysplastic syndromes (MDS)). Thus, the use of ILC1s provides a previously unknown strategy to treat cancer (e.g., AML) and/or prevent relapse of the disease.

ILC1s play critical roles in inflammation and in the early anti-viral response^{40,41}. However, the role of ILC1s in preventing and/or promoting cancer, including AML, has not been explored⁴². In particular, it is unknown whether ILC1s suppress or promote cancer development. Described herein are in vitro studies as well as three different mouse models that show that the progression of AML can be controlled by ILC1s. Without being bound by theory, this is accomplished by ILC1 directly interacting with LSCs. ILC1s play dual roles in regulating LSCs, particularly in AML: 1) ILC1s induce apoptosis of LSCs; and 2) ILC1s suppress differentiation of LSCs into leukemia progenitor cells, facilitate differentiation of LSCs into non-leukemic cells, and block differentiation of LSCs into myeloid blasts. Without being bound by theory, IFN-γ mediates ILC1-induced effects on LSCs via both the JAK-STAT and PI3K-AKT signaling pathways.

As shown herein, high concentration of normal murine ILC1s induced leukemia stem cell (LSC) apoptosis. At a lower concentration, ILC1s prevented LSCs from differentiating into leukemia progenitors and promoted their differentiation into non-leukemic cells, thus blocking the production of terminal myeloid blasts. Without being bound by theory, these effects are mediated by ILC1s' ability to produce interferon-γ after cell-cell contact with LSCs. ILC1s also displayed to suppress leukemogenesis in vivo, and thus in some embodiments, disclosed herein are methods of suppress leukemogenesis comprising administering to a patient in need thereof a therapeutically effect amount of a composition comprising a population of ICL1s. In some embodiments, disclosed herein are methods of using a population of ILC1s described herein to prolong relapse-free survival in AML, prevent relapse of AML, and/or reduce the chance of relapse of AML.

AML is a highly heterogeneous and aggressive malignancy. The most commonly used therapies are chemotherapy followed by allogeneic stem cell transplantation. However, among patients who relapse, there exists a small population of leukemia-initiating cells or LSCs that ultimately proved resistant to therapy^{4,43}. Thus, developing novel approaches to targeting LSCs offers a potential strategy to prolong relapse-free survival of AML patients. Chemotherapy and targeted therapy (e.g., tyrosine kinases inhibitors including the Food and Drug Administration (FDA)-approved drugs midostaurin and gilteritinib) can kill leukemic blasts but may also enrich LSCS^{44,45}. As described herein, ILC1s act directly on LSCs, resulting in reduced progression of AML in vivo. Thus, expanding ILC1 cells ex vivo during times of remission or combining expanded ILC1s with an FDA-approved drug that enriches LSCs, may have a positive impact on prolonging relapse-free survival of AML patients. The methods described herein can be used alone or in combination with other treatments and methods used and known in the art to treat AML, ameliorate a symptom of AML, prolong relapse-free survival in AML, prevent or reduce the chance of relapse of AML, or kill or reduce LSCs or leukemic blasts.

IFN-γ plays important roles in anti-viral and anti-tumor immunity and has been used clinically to treat several diseases⁴⁶. However, IFN-γ-based therapies have at least two limitations in the clinic that preclude routine use for the treatment of cancer patients. The first limitation is that IFN-γ cannot be delivered into local tumor sites and subsequently achieve effective concentrations in the TME (tumor microenvironment) without causing significant toxicities⁴⁷⁻⁴⁹; the second limitation is that IFN-γ is rapidly cleared from the blood after intravenous administration, further limiting the ability to achieve effective local concentrations. These disadvantages in the clinical use of IFN-γ necessitate the development of alternative methods to ensure its effectiveness in the local milieu of the marrow while limiting its toxicity. Thus, in some embodiments, the methods described herein increase the IFN-γ concentration in the TME.

Described herein, inter alia, are methods of treating AML by utilizing a cell-based source of IFN-γ to target LSCs. Although ILC1s are a minute cell population, they express abundant IFN-γ, especially when they interact with tumor cells in the TME. ILC1s also express high levels of chemokine receptors including CXCR3 and CXCR6, the receptors for CXCL9-11 and CXCL16, respectively, that are expressed by AM cells^{41,50}. Without being bound by theory, these receptor-ligand interactions may help recruit ILC to the bone marrow or tumor sites, where the majority of LSCs reside⁵¹. Also described herein, ILC1s rapidly and persistently produce IFN-γ locally (e.g. within the TME) after contacting LSCs or more mature tumor cells, yielding sufficient doses of the cytokine to target and kill AML blasts⁵². Also described herein, ILC1s induce apoptosis and differentiation of LSCs within the TME. Moreover, ILC1s are associated with reducing severe progression of graft-versus-host disease (GVHD) after allogeneic HSCT in AML patients⁵³. This suggests that ILC can control AML in different layers and at different settings through their multifaceted roles.

In some embodiments, provided herein are methods to rapidly and reproducibly expand ILC and the use of ILC for application as a cellular therapy (e.g., prolong relapse-free survival in AML patients who achieve complete remission but may carry quiescent LSCs, especially for patients ineligible for HSCT).

The IFN-γ signaling pathway is associated with several biological responses and plays an important role in innate and adaptive immunity. It can not only induce apoptosis of tumor cells⁵¹, but also activate immune cells, two processes that are crucial for defending against cancer^{46,55}. IFN-γ induces PD-L1 expression in tumor cells including AML blast cells⁵⁶ and immune cells^{57,58}; it regulates PD-L1 expression mainly through the JAK1/2-STAT1/3-IRF1 axis in melanoma cells⁵⁹. As described herein, ILC1s and recombinant IFN-γ block differentiation of LSCs into leukemia progenitor cells. The action of IFN-γ on tumors, tumor stem cells, and immune cells can induce PD-L1 expression, which can block T cell responses to tumor cells and their stem cells⁶⁰, differentiation of cancer stem cells, and activation of immune cells⁶¹. The use of IFN-γ should consider all of these effects, the ability of an anti-PD-L1 antibody to block the adverse effects of IFN-γ-upregulated PD-L1. In some embodiments, the methods described herein can be sued alone or in combination with IFN-γ, cells that produce this cytokine, or mimetics thereof. In some embodiments, the methods described herein (e.g., a method of treating AML using ILC1s) can be combined with administering anti-PD-L1 antibody. The innovative methods described herein (e.g. leukemia treatment) may bring new hope to patients with AML, especially relapsed older patients who otherwise may live only for several months.

Disclosed herein, inter alia, are compositions comprising ILC1s to treat AML and regulate LSCs by inducing apoptosis, inhibiting LSC differentiation into leukemia progenitors cells, promoting LSC differentiation into a non-leukemic lineage, blocking differentiation into myeloid blasts, and increasing and prolonging IFN-γ concentrations in the TME. Also described herein is are methods of treatment comprising ILC1 cell therapy (e.g., to prolong relapse-free survival of patients diagnosed with AML).

Described herein, inter alia, are method of preparing isolated ILC1 cells, methods of preparing ex vivo expanded ILC1 cells (i.e., human ILC1 cells), and compositions comprising each. In some embodiments, described herein is a method comprising:
(a) isolating a population of type I innate lymphoid cells (ILC1s); and
(b) culturing the population of ILC1s in growth media under conditions and for a time to expand the population of ILC1s.

In some embodiments, the population of ILC1s are human. In some embodiments, the population of ILC1s are from a mouse or other mammal. In some embodiments, the population of ILC1s are isolated from blood, peripheral blood, or peripheral blood mononuclear cells (PBMCs) and are autologous to patient that is to be administered the cells. In some embodiments, the population of ILC1s comprise 30%, 40% 50%, 60%, 70%, 80%, 85%, 90%, 95%, 96%, 97%, 98%, 99%, or 100% ILC1s. In some embodiments, the population of ILC1s comprise at least 50%, at least 60%, at least 70%, at least 80%, at least 85%, at least 90%, at least 95%, at least 96%, at least 97%, at least 98%, or at least 99% ILC1s. In some embodiments, the population of ILC1s comprise cells selected from:
Lin⁻CD56⁻CD127⁺c-Kit⁻CRTH2⁻,
Lin⁻CD56⁻CD127⁺,
Lin⁻CD56⁻CD127⁺c-Kit⁻,
Lin⁻CD56⁻CD127⁺c-Kit⁻CRTH2⁻EOMES⁻,
Lin⁻CD56⁻CD127⁺c-Kit⁻CRTH2⁻CXCR3⁺CXCR6⁺, or
Lin⁻CD56⁻CD127⁺c-Kit⁻CRTH2⁻EOMES⁻CXCR3⁺CXCR6⁺.

In some embodiments the population of cells comprises ILC1s that are:
at least 90%, 95% or 98% Lin⁻CD56⁻CD127⁺c-Kit⁻CRTH2⁻,
at least 90%, 95% or 98% Lin⁻CD56⁻CD127⁺,
at least 90%, 95% or 98% Lin⁻CD56⁻CD127⁺c-Kit⁻,
at least 90%, 95% or 98% Lin⁻CD56⁻CD127⁺c-Kit⁻CRTH2⁻EOMES⁻,
at least 90%, 95% or 98% Lin⁻CD56⁻CD127⁺c-Kit⁻CRTH2⁻CXCR3⁺CXCR6⁺, or
at least 90%, 95% or 98% Lin⁻CD56⁻CD127⁺c-Kit⁻CRTH2⁻EOMES⁻CXCR3⁺CXCR6⁺.

In some embodiments, the population of ILC1s is contacted with at least one of IL-2, IL-12, IL-15, or IL-7 (preferably human IL-2, IL-12, IL-15, or IL-7). In some embodiments, the isolated population of ILC1s is co-cultured with feeder cells. In some embodiments, the feeder cells comprise 721.221 cells or K562 cells. In some embodiments, the ILC1:feeder cell ratio is 1:1, 1:2, 1:3, 1:4, 1:5, 2:1, 3:1, 4:1, or 5:1.

Also described herein is an isolated population of ILC1 cells, wherein at least 50%, at least 60%, at least 70%, at least 80%, at least 85%, at least 90%, at least 95%, at least 96%, at least 97%, at least 98%, or at least 99% of the cells are selected from:
Lin⁻CD56⁻CD127⁺c-Kit⁻CRTH2⁻,
Lin⁻CD56⁻CD127 +,
Lin⁻CD56⁻CD127⁺c-Kit⁻,
Lin⁻CD56⁻CD127⁺c-Kit⁻CRTH2⁻EOMES⁻,
Lin⁻CD56⁻CD127⁺c-Kit⁻CRTH2⁻CXCR3⁺CXCR6⁺, or
Lin⁻CD56⁻CD127⁺c-Kit⁻CRTH2⁻EOMES⁻CXCR3⁺CXCR6⁺.

Also described herein are compositions comprising a population of isolated ILC1s, a population of ex vivo expanded ILC1s, or a population of ILC prepared by any of the methods described herein.

Provided herein, inter alia, is a method of treating a cancer or leukemia, the method comprising administering to a subject in need thereof a population of isolated ILC1s, a population of ex vivo expanded ILC1s, a population of ILC1s prepared by any of the methods described herein, a composition described herein, or a composition comprising any of the populations of ILC1 described herein.

Provided herein, inter alia, is a method of killing, eliminating, or reducing cancer cells, leukemia cells, leukemia stem cells (LSCs), leukemia progenitor cells, myeloid blasts, or cells expressing CXCL9-11 or CXCL16, the method comprising administering to a subject in need thereof a population of isolated ILC1s, a population of ex vivo expanded ILC1s, a population of ILC prepared by any of the methods described herein, a composition described herein, or a composition comprising any of the populations of ILC1 described herein.

Provided herein, inter alia, is a method of reducing or ameliorating a symptom associated with a cancer or leukemia, the method comprising administering to a subject in need thereof a population of isolated ILC1s, a population of ex vivo expanded ILC1s, a population of ILC prepared by any of the methods described herein, a composition described herein, or a composition comprising any of the populations of ILC1 described herein.

Provided herein, inter alia, is a method of inhibiting or reducing leukemogenesis, the method comprising administering to a subject in need thereof a population of isolated ILC1s, a population of ex vivo expanded ILC1s, a population of ILC1s prepared by any of the methods described herein, a composition described herein, or a composition comprising any of the populations of ILC1 described herein.

Provided herein, inter alia, is a method of inhibiting or reducing differentiation of LSCs into leukemia progenitor cells or myeloid blasts, the method comprising administering to a subject in need thereof a population of isolated ILC1s, a population of ex vivo expanded ILC1s, a population of ILC prepared by any of the methods described herein, a composition described herein, or a composition comprising any of the populations of ILC1 described herein.

Provided herein, inter alia, is a method of promoting or increasing differentiation of LSCs to non-leukemic cells, the method comprising administering to a subject in need thereof a population of isolated ILC1s, a population of ex vivo expanded ILC1s, a population of ILC prepared by any of the methods described herein, a composition described herein, or a composition comprising any of the populations of ILC1 described herein.

Provided herein, inter alia, is a method of prolonging relapse-free survival, preventing relapse, or decreasing the risk of relapse in a cancer or leukemia patient, the method comprising administering to a subject in need thereof a population of isolated ILC1s, a population of ex vivo expanded ILC1s, a population of ILC prepared by any of the methods described herein, a composition described herein, or a composition comprising any of the populations of ILC1 described herein.

Provided herein, inter alia, is a method of increasing prolonging INF-γ concentration or prolonging INF-γ presence in a tumor microenvironment, the method comprising administering to a subject in need thereof a population of isolated ILC1s, a population of ex vivo expanded ILC1s, a population of ILC1s prepared by any of the methods described herein, a composition described herein, or a composition comprising any of population of ILC1 described herein.

In some embodiments, the isolated ILC or ex vivo expanded ILC are human. In some embodiments, the isolated ILC1s or ex vivo expanded ILC1s are autologous or allogenic. In some embodiments, the autologous ILC1s are isolated from the patient during remission or any cancer free time. In some embodiments, the population of isolated ILC1s or ex vivo expanded ILC1 cells or a composition described herein is administered in single or repeat dosing. In some embodiments, an effective amount of the population of isolated ILC or ex vivo expanded ILC1 cells or a composition described herein is administered.

In some embodiment, the population of isolated ILC or ex vivo expanded ILC1 cells or a composition described herein is administered locally or systemically. In some embodiments, the population of isolated ILC1s or ex vivo expanded ILC1s or a composition described herein is infused or administered intravenously, locally or directly injected, injected into tumor microenvironment, or administered intratumorally. In some embodiments, at least one symptom of a cancer or leukemia is reduced, ameliorated, or relieved. In some embodiments, the leukemia is any of acute lymphocytic leukemia (ALL), acute myeloid leukemia (AML), chronic lymphocytic leukemia (CLL), chronic myeloid leukemia (CML), hairy cell leukemia (HCL), or myelodysplastic syndromes (MDS). In some embodiments, the population of isolated ILC1s or ex vivo expanded ILC1 cells or a composition described herein is administered before remission, during remission, or during relapse. In some embodiments, the population of isolated ILC1s or ex vivo expanded ILC1 cells or a composition described herein is administered before, after, or in combination with one or more of IFN-γ (or a nucleic acid encoding IFN-γ), a cytokine (or a nucleic acid encoding a cytokine), IL-15 (or a nucleic acid encoding IL-15), an anti-PD-L1 antibody or a PD-L1 inhibitor, an anti-PD-1 antibody or a PD-1 inhibitor, a chemotherapy, a kinase inhibitor (e.g., midostaurin and gilteritinib), or radiation therapy.

Also described herein are ILC1s harboring a recombinant nucleic acid molecule encoding a protein of interest.

For example, the recombinant nucleic acid can encode human IL-15 (Gene ID: 3600; GenBank® Accession: NP 000576.1). For example, it can encode amino acids 1-162, 30-162, 49-162 or a functional portion thereof of SEQ ID NO: 1

| (SEQ ID NO: 1) | |
|---|---|
| 1 | mriskphlrs isiqcylcll lnshflteag ihvfilgcfs aglpkteanw vnvisdlkki |
| 61 | edliqsmhid atlytesdvh psckvtamkc fllelqvisl esgdasihdt venliilann |
| 121 | slssngnvte sgckeceele eknikeflqs fvhivqmfin ts |

For example the recombinant nucleic acid can encode human IL-12 (IL-12 subunit A: Gene ID: 3592; GenBank® Accession: NP 000873 and IL-12 subunit B Gene ID: 3593; GenBank® Accession: NM 002187.2). For example, it can encode amino acids 1-253, 57-253 or a functional portion thereof of SEQ ID NO: 2

| (SEQ ID NO: 2) | |
|---|---|
| 1 | mwppgsasqp ppspaaatgl hpaarpvslq crlsmcpars lllvatlvll dhlslarnlp |
| 61 | vatpdpgmfp clhhsqnllr avsnmlqkar qtlefypcts eeidheditk dktstveacl |
| 121 | pleltknesc lnsretsfit ngsclasrkt simmalclss iyedlkmyqv efktmnakll |
| 181 | mdpkrqifld qnmlavidel mqalnfnset vpqkssleep dfyktkiklc illhafrira |
| 241 | vtidrvmsyl nas |

and amino acids 1-328, 23-328 or a functional portion thereof of SEQ ID NO: 3).

| (SEQ ID NO: 3) | |
|---|---|
| 1 | mchqqlvisw fslvflaspl vaiwelkkdv yvveldwypd apgemvvltc dtpeedgitw |
| 61 | tldqssevlg sgktltiqvk efgdagqytc hkggevlshs llllhkkedg iwstdilkdq |
| 121 | kepknktflr ceaknysgrf tcwwlttist dltfsvkssr gssdpqgvtc gaatlsaerv |
| 181 | rgdnkeyeys vecqedsacp aaeeslpiev mvdavhklky enytssffir diikpdppkn |
| 241 | lqlkplknsr qvevsweypd twstphsyfs ltfcvqvqgk skrekkdrvf tdktsatvic |
| 301 | rknasisvra qdryysssws ewasvpcs |

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-1I show ILC1 induces leukemia stem cell apoptosis. (A) shows pictures of mouse LSCs (Lin⁻Scα-1⁺c-Kit⁺) from the spleen of Mll^{PTD/WT:Flt}3^{ITD/ITD} were co-cultured with or without mouse ILC1s (magnification is 10, n=3). (B) shows flow cytometry plots and statistics of the percentage of apoptotic cells in LSCs co-cultured with or without ILC1s (n=3). (C) shows a bar graph of the percentage of caspase⁺apoptotic cells in LSCs co-cultured with or without ILC1s (n=3). (D) shows pictures of human LSCs (Lin⁻CD34⁺CD38⁻) from blood of AML patient co-cultured with or without human ILC (Lin⁻CD56⁻CD127⁺c-Kit⁻CRTH2⁻) (magnification is 10, n=3). (E) shows flow cytometry plots and statistics of the percentage of apoptotic cells in LSCs after human LSCs isolated from blood of AML patients co-cultured with or without human ILC1s (n=3). (F) shows a bar graph of the percentage of caspase +apoptotic cells of LSCs after human LSCs from AML patient blood labeled by Cell Trace™ Far Red dye were co-cultured with or without human ILC1s (n=3). (G) shows a bar graph of the percentage of apoptotic cells in LSCs after mouse LSCs were co-cultured with or without mouse ILC in the presence or absence of mouse anti-IFN-γ or TNF-α (n=3). (H) shows flow cytometry plots of the percentage of apoptotic cells of LSCs after human LSCs from blood of AML patients were co-cultured with or without human ILC in the presence or absence human anti-IFN-γ. (I) shows a bar graph of the percentage of anti-FN-γ⁺and anti-TNF-α⁺ILC1s (n=3). Mouse LSCs were transferred into the top well of a 96-well transwell plate. Bottom chambers of transwell plates were loaded with or without ILC1s in the presence or absence of IL-12+IL-15. BD GolgiPlug™ was added to the cultures at incubation onset. All data shown as mean±SD. P values were calculated by one-way ANOVA. *p<0.05, **p<0.01, ****p<0.0001.

FIGS. 2A-2J show ILC1 and ILC1-secreted-IFN-γ inhibits differentiation of LSCs. (A-C) show lines graphs of the percentage of Lin⁻Scα-1⁺c-Kit⁺, Lin⁻Scα-1⁻c-Kit⁺, and Lin⁻Scα-1⁺c-Kit⁻respectively following mouse LSCs labeled by CTV were co-cultured with or without mouse ILC1s. Statistics are shown (n=3). (D-E) shows flow cytometry plots and a bar graph of the percentage of Lin⁻Scα-1⁺c-Kit⁺, Lin⁻Scα-1⁻c-Kit⁺, and Lin⁻Sca-1⁺c-Kit⁻of mouse LSCs labeled by CTV that were co-cultured with or without mouse ILC in the presence or absence of anti-IFN-γ or TNF-α (n=4). (F) shows flow cytometry plots of the percentage of Lin⁻Scα-1⁺c-Kit⁺, Lin⁻Scα-1⁻c-Kit⁺, and Lin⁻Scα-1⁺c-Kit⁻cells after mouse LSCs labeled by CTV were co-cultured with or without mouse WT, TNF-α^{-/-} ILC (n=3). (G-H) show line graphs of the percentage of Lin⁻Scα-1⁺c-Kit⁺and Lin⁻Scα-1⁻c-Kit⁺cells after mouse LSCs labeled by CTV were co-cultured with or without mouse WT, IFN-γ^{-/-} or TNF-α^{-/-} ILC1s (n=3). (I) shows flow cytometry plots of the percentage of Lin⁻Scα-1⁺c-Kit⁺, Lin⁻Scα-1⁻c-Kit⁺, and Lin⁻Scα-1⁺c-Kit⁻cells after mouse LSCs were co-cultured with or without 0.1 ng/ml, 1 ng/ml and 10 ng/ml recombinant murine IFN-γ (n=3). (J) is a schematic depiction showing ILC1s were transferred into the top well of a 96-well transwell plate, bottom chambers of transwell plates were loaded with LSCs from the spleen of Mll^{PTD/WT}:Flt3^{ITD/ITD} mice and then co-cultured for 3 days. Flow cytometry plots of the percentage of Lin⁻Scα-1⁺c-Kit⁺, Lin⁻Scα-1⁺, and Lin⁻Scα-1⁺c-Kit⁻cells are also shown (n=3). The data of (2J-21) with statistics are shown in FIGS. 8A-8F. All data shown as mean±SD. P values were calculated by either one-way ANOVA or student's t test. *p<0.05, **p<0.01, ****p<0.0001.

FIGS. 3A-3I show ILC1 inhibits differentiation of LSCs into mature blasts. (A-B) show line graphs of the percentage of CD11b⁺and Gr-1⁺cells after mouse LSCs labeled by CTV were co-cultured with or without mouse ILC1s (n=3). (C-D) show bar graphs of the percentage of CD11b⁺and Gr-1⁺cells after mouse LSCs labeled by CTV were co-cultured with or without mouse WT, TNF-γ^{-/-} or TNF-α^{-/-} ILC1s (n=3). (E) shows images of Wright-Giemsa staining of cells after mouse LSCs labeled by CTV were co-cultured with or without mouse WT, IFN-γ^{-/-} or TNF-α^{-/-} ILC1s (magnification is 20, n=3). (F) is a bar graph that shows colony-forming cell counts at each round of plating after mouse LSCs labeled by CTV were co-cultured with or without mouse WT, IFN-γ^{-/-} or TNF-α^{-/-} ILC1s (n=3), triplications for each of them. (G-H) are bar graphs of the percentage of Mac-1⁺and Gr-1⁺cells after mouse LSCs were co-cultured with or without 0.1 ng/ml, 1 ng/ml and 10 ng/ml recombinant murine IFN-γ. (I) is a heatmap showing RNA differential expression of terminal myeloid differentiation and lymphoid differentiation genes (n=3). All data shown as mean±SD. P values were calculated by either one-way ANOVA or student's t test. *p<0.05, **p<0.01, ****p<0.0001.

FIGS. 4A-4N show the survival of leukemia mice after treatment with ILC1s and/or IFN-γ. 3×10⁴ LSCs were intravenously injected into non-lethally irradiated (200 cGy) immunocompromised Rag2^{-/-}γc^{-/-} recipient mice on day 0. Mice were intravenously injected with 3×10⁴ mouse ILC1s from the livers of C57BL/6J (CD45.2) mice on day 1, 8, 15 and 22. Total white blood cells (WBCs; b), neutrophils (c), and monocytes (d) were measured at weeks 3, 6, 9 (n=9). (e) Schematic of the design and procedures for (f-h). Bone marrow cells were isolated from IL-15 transgenic mice and depleted of NK1.1⁺TKp46⁺cells, and 3×10⁴ LSCs sorted from spleens of Mll^{PTD/WT}:Flt3^{ITD/ITD} mice were intravenously co-injected into lethally irradiated (900 cGy) C57BL/6J (CD45.2) recipient mice on day 0. Mice were intravenously injected with 3×10⁴ ILC1s or 0.5 μg recombinant murine IFN-γ on day 1. Total WBCs (f), neutrophils (g), and monocytes (h) were measured at week 5 (n=6). (i) Schematic of the design and procedures for (J-L). 3×10⁴ LSCs from the spleens of Mll^{PTD/WT}:Flt3^{ITD/ITD} mice were intravenously injected into lethally irradiated (900 cGy) C57BL/6J (CD45.2) recipient mice on day 0. Mice were intravenously injected with 3×10⁴ mouse ILC1s from the livers of WT or IFN-γ^{-/-} mice on day 1. Total WBCs (j), neutrophils (k), and monocytes (l) were measured at week 6 (n=5-8). (M) Images of peripheral blood smears are shown (n≥3 for each group). (N) Kaplan-Meier survival analysis for mice injected with or without WT ILC1, or IFN-γ^{-/-} ILC1s by Kaplan-Meier method and log-rank test (n=7-9). All non-survival data are shown as mean 35 SD. P values were calculated by either one-way ANOVA models or linear mixed models. *p<0.05, **p<0.01, ****p<0.0001, ns, not significant.

FIGS. 5A-5H show ILC1s effect on myeloid differentiation of LSCs through the JAK-STAT and PI3K-AKT signaling pathways. (A) shows a heatmap of RNA differential expression of 627genes after FACS-sorted mouse LSCs were treated with FACS-sorted ILC1s or IFN-γ for 3 days (n=3). LSCs were then re-sorted for RNA-seq. (B) shows a pathway analysis in LSCs RNA pools (ILC1 vs. CTRL; control=no treatment). Left panel shows downregulated signaling pathways in LSCs. Right panel showed upregulated signaling pathways (n=3). (C) shows GSEA plots showing enrichment of some target genes in AML LSCs after co-cultured with ILC1s. The X-axis represents the rank ordering (ILC1 vs. CTRL) of all genes (n=3). (D) is a bar graph showing RNA differential expression of Akt3. Results are expressed as means compared with CTRL (n=3). (E) is a bar graph showing RNA differential expression of Jak2. Results are expressed as means compared with CTRL (n=3). (F) is a bar graph showing RNA differential expression of Stat1 and Stat2 (n=3). (G-H) are box plots showing flow cytometry statistics of the percentage of Lin⁻Scα-1⁺c-Kit⁺, Lin⁻Scα-1⁻c-Kit⁺cells, and Lin⁻Scα-1⁺c-Kit⁻cells after mouse LSCs labeled by CTV were treated with or without JAK and AKT inhibitors for 30 min and then co-cultured with WT and IFN-γ^{-/-} ILC1s in the presence of IL-12 plus IL-15 for 3 days (n=4). All data shown as mean ±SD. P values were calculated by either one-way ANOVA or student's t test. *p<0.05, **p<0.01, ****p<0.0001.

FIGS. 6A-6C show the function of ILC1s in AML mouse. 2×10⁶ C1498 cells were intravenously injected into C57BL/6J mice for 21 days. (A-B) are bar graphs showing the production of TNF-α and IFN-γ in liver ILC1s isolated from normal or AML mice are shown (n=3-4). (C) is a GSEA plot showing the relative abundance of genes involved in the TNF-α/NF-κB signaling pathways in liver ILC1s isolated from normal or AML mice. All data are shown as mean±SD. P values were calculated by student's t-test. *p<0.05, **p<0.01, ****p<0.0001.

FIGS. 7A-7C show IFN-γ induction of LSC apoptosis. (A) shows a schematic depiction of the experimental set up in the transwell plate and a bar graph of the percentage and statistics of apoptotic cells in LSCs. 5,000-10,000 murine ILC1s from liver were sorted by BD FACSAria™ Fusion Cell Sorter. The sorted ILC1s were transferred into the top well of a 24-well transwell plate. Bottom chambers of transwell plates were loaded with LSCs from the spleen of Mll^{PTD/WT}:Flt3^{ITD/ITD} mice and then co-cultured for 4 days. All data are shown as mean±SD. P values were calculated by one-way ANOVA. *p<0.05, **p<0.01, ****p<0.0001. (B-C) show images (top, magnification is 10) and flow cytometry plots (bottom) of the percentage of apoptotic cells in LSCs. Mouse LSCs from the spleen of Mll^{PTD/WT}:Flt3^{ITD/ITD} mice were treated with different doses of IFN-γ and TNF-α for 4 days.

FIGS. 8A-8F show the percentage of Lin⁻Scα-1⁺c-Kit⁺, Lin⁻Scα-1⁻c-Kit⁺and Lin⁻Scα-1⁺c-Kit⁻after IFN-γ or ILC1 treatment. (A-C) show bar graphs of the percentage of Lin⁻Scα-1⁺c-Kit⁺, Lin⁻Scα-1⁺c-Kit⁺, and Lin⁻Scα-1⁺c-Kit⁻cells after mouse LSCs from the spleen of Mll^{PTD/WT}:Flt3^{ITD/ITD} mice were co-cultured with or without 0.1 ng/ml, 1 ng/ml or 10 ng/ml recombinant murine IFN-γ (n=3). (D-F) show bar graphs of the percentage of Lin⁻Scα-1⁺c-Kit⁺, Lin⁻Scα-1⁻c-Kit⁺, and Lin⁻Scα-1⁺c-Kit⁻cells after LSCs from the spleen of Mll^{PTD/WT}:Flt3^{ITD/ITD} mice were co-cultured with or without ILC1s. ILC1s from mouse liver were sorted by an BD FACSAria™ Fusion Cell Sorter and transferred into the top well of a 96-well transwell plate. Bottom chambers of transwell plates were loaded with LSCs from the spleen of Mll^{PTD/WT}:Flt3^{ITD/ITD} mice and then co-cultured for 3 days (n=3). All data shown as mean±SD. P values were calculated by either one-way ANOVA or student's t test. *p<0.05, **p<0.01, ****p<0.0001.

FIGS. 9A-9D show the percentage of Mac-1⁺and Gr-1⁺cells after treatment with WT ILC1, IFN-γ^{-/-} ILC1, or IFN-γ(A-C) show cytometry plots and statistics of the percentage of Mac-1⁺and Gr-1⁺cells after LSCs from the spleen of Mll^{PTD/WT}:Flt3^{ITD/ITD} mice were co-cultured with or without WT ILC1, IFN-γ^{-/- ILC}1, or IFN-γ. (D) shows a schematic depiction illustrating the role of ILC and ILC1s-derived IFN-γ in regulating differentiation of LSCs.

FIG. 10 shows the absolute numbers of WBCs, monocytes, and neutrophils in AML mice. On day 0, 3×10⁴ LSCs from spleen of Mll^{PTD/WT}:Flt3^{ITD/ITD} mice were intravenously injected into 900 cGy irradiated CD45.2 receipt mice. On day 1, 3×10⁴ WT or IFN-γ^{-/-} mouse ILC from liver of CD45.2 were intravenously injected into those mice. The absolute numbers of WBCs, neutrophils, and monocytes were tested at week 3 (n=7). All data are shown as mean±SD. P values were calculated by either one-way ANOVA or student's t test. *p<0.05, ** p<0.01, ****p<0.0001.

FIGS. 11A-11F show ILC1 and ILC1-derived IFN-γ effect on LSC differentiation and the JAK-STAT and AKT signaling pathways. (A) is a volcano plot of differentially expressed genes from AML LSCs (IFN-γ vs. ILC1s) RNA pools. (B) shows the Hallmark pathway analysis in LSCs RNA pools (IFN-γ vs. Ctrl; Ctrl means no treatment). The left panel shows downregulated signaling pathways in LSCs. The right panel shows upregulated signaling pathways. (C) shows GSEA plots showing enrichment of some target genes in LSCs after co-cultured with ILC1s. The

X-axis represents the rank ordering (ILC1s vs. Ctrl) of all genes (Ctrl means no treatment). (D) shows GSEA plots showings enrichment of some target genes in LSCs after co-cultured with IFN-γ. The X-axis represents the rank ordering (IFN-γ vs. Ctrl) of all genes. (E-F) are heatmaps showing RNA differential expression of downstream genes of IFN-γ. All data are shown as mean±SD. P values were calculated by either one-way ANOVA or student's t test. *p<0.05, **p<0.01, ****p<0.0001.

FIGS. 12A-12E show the gating strategy for slow cytometry analysis of LSCs apoptosis, human ILC1s, and mouse ILC1s. (A) shows the gating strategy for flow cytometry analysis of apoptosis of LSCs co-cultured with ILC1s using 7-AAD. CTV, CellTrace™ Violet. FMO, fluorescence minus one. (B) shows the gating strategy for flow cytometry analysis of apoptosis of LSCs co-cultured with ILC1s using Violet Live Cell Caspase Probe. (C) shows the gating strategy for flow cytometry analysis of the human ILC1s isolated from peripheral blood. Lineage (Lin): CD3, CD4, CD8, CD14, CD15, CD16, CD19, CD20, CD33, CD34, CD203c, and FceRI. Human ILC1s were defined as Lin⁻CD56⁻CD127⁺CRTH2⁻. (D) shows the gating strategy for flow cytometry analysis of differentiation of mouse LSCs co-cultured with WT, IFN-γ^{-/-}, or TNF-α^{-/-} ILC1s. Lineage (Lin): CD3, CD19, B220, CD11b, Ly6G/C and Ter119. Mouse LSCs were defined as Lin⁻Scal-1⁺c-Kit⁺. (E) shows the gating strategy for flow cytometry analysis of human LSCs. Lineage (Lin): CD2, CD3, CD4, CD8, CD14, CD16, CD19, CD11b, CD56 and CD235a. Human LSCs were defined as Lin⁻CD34⁺CD38⁻.

FIGS. 13A-13B show cellular expansion of ILC1s. (A) is a line graph of the fold change of ILCs after stimulation/activation up to seven days. (B) is a bar graph showing fold change of ILCs after stimulation/activation at 14 days and 21 days.

FIGS. 14A-14O show ILC1s induce apoptosis in leukemia stem cells. (A) depicts the experimental design for detecting LSC apoptosis in vitro. Mouse LSCs (Lin⁻Sca-1⁺c-Kit⁺) from the spleen of Mll^{PTD/WT}: Flt3^{ITD/ITD} mice with AML or human LSCs (Lin⁻CD45^{dim}CD34⁺CD38⁻) from blood of patients with AML labeled with Cell Trace Violet (CTV) were co-cultured with or without the mouse or human ILC1s for 3 days. (B) shows images (left; 5× magnification, scale bar, 200 ρm) and statistics of absolute cell numbers (right) (n=3). (C) shows flow cytometry plots (left) and statistics of the percentages of apoptotic cells (right) in mouse LSCs co-cultured with or without mouse ILC1s (n=3). (D) shows percentages of caspase +apoptotic cells in mouse LSCs co-cultured with or without mouse ILC1s for 1 day (n=3). (E) shows caspase 3/7 activity in mouse LSCs after co-culture with or without mouse ILC1s for 6 h. Results are expressed as fold changes compared to co-culture without ILC1s (n=4). (F) shows qRT-PCR analyses for Bak1 gene in mouse LSCs co-cultured with or without mouse ILC1s for 6 h (n=3). LSCs were separated from co-cultured ILC1s using FACS and then analyzed with qRT-PCR. (G) shows human LSCs (Lin⁻CD45^{dim}CD34⁺CD38⁻) from blood of patients with AML were co-cultured with or without human ILC1s (Lin⁻CD56⁻CD127⁺c-Kit⁻CRTH2⁻) for 3 days. The images (left; 5× magnification, scale bar, 200 μm) and statistics of absolute cell numbers (right) (n=3). (H) shows flow cytometry plots (left) and statistics of the percentages of apoptotic cells (right) in human LSCs (n=3). (I) shows percentages of caspase⁺apoptotic cells in human LSCs co-cultured with or without human ILC1s for 1 day (n=3). (J) shows caspase 3/7 activity in human LSCs after co-culture with or without human ILC for 6 h. Results are expressed as fold changes compared to co-culture without human ILC (n=3). (K) shows qRT-PCR analyses for Bak1 gene in human LSCs co-cultured with or without human ILC1s for 6 h (n=3). LSCs were separated from co-cultured ILC using FACS and then analyzed with qRT-PCR. (L) shows mouse LSCs were co-cultured with or without mouse ILC1s for 3 days in the presence or absence of mouse anti-IFN-γ or anti-TNF-α. The images (left; 5× magnification, scale bar 200 μm) and statistics of absolute cell numbers (right) are shown (n=3). (M) shows the relative percentages of apoptotic LSCs were compared statistically. (n=3). (N) shows human LSCs from blood of patients with AML were co-cultured with or without human ILC for 3 days in the presence or absence of human anti-IFN-γ. Flow cytometry plots (left) and statistics of the percentages of apoptotic cells (right) in human LSCs (n=3). (O) shows mouse ILC1s were loaded into the bottom chambers of a 96-well Transwell plate. The top wells of the plate were loaded with or without LSCs in the presence of IL-12+IL-15 cytokines. BD GolgiPlug™ was added to the cultures at incubation, and percentages of IFN-γ⁺ILC1s were calculated (n=3). Data are presented as mean±s.d.; P values were calculated by either Student's t test or one-way ANOVA models. *P <0.05; **P <0.01; ***P <0.001; ****P <0.0001; NS, not significant.

FIGS. 15A-15M show IFN-γ secreted by ILC1s inhibits the differentiation of LSCs into leukemia progenitors and promotes their differentiation into non-leukemic cells. (A-E) Mouse LSCs labeled with CTV were co-cultured with or without mouse ILC1s. Flow cytometry plots (A), statistics of absolute cell numbers (B-E; top) and percentages (B-E; bottom) of Lin⁻Sca-1⁺c-Kit⁺, Lin⁻Sca-1⁺c-Kit⁻, and Lin⁻Sca-1⁻c-Kit⁻ cells are shown (n=4). (F-J) Mouse LSCs labeled with CTV were co-cultured with or without WT, IFN-γ^{-/-}, or TNF-α^{-/-} IL1s in the presence or absence of anti-IFN-γ or anti-TNF-α antibody. Flow cytometry plots (F), statistics (G-J) of absolute cell numbers (left) as well as percentages (right) of Lin⁻Sca-1⁺c-Kit⁺, Lin⁻Sca-1⁻Lin⁻Sca-1⁺c-Kit⁺, and Lin⁻Sca-1⁻c-Kit⁻ cells (n=4). (K-L) Mouse LSCs were co-cultured with or without 0.1 ng/ml, 1 ng/ml, or 10 ng/ml recombinant murine IFN-γ. Flow cytometry plots (K) and statistics of absolute cell numbers (L) of Lin⁻Sca-1⁺Lin⁻Sca-1⁻c-Kit⁺, and Lin⁻Sca-1⁺c-Kit⁻ cells are shown (n=3). (M) ILC1s were transferred into the top well of a 96-well Transwell plate, the bottom chambers of the plate were loaded with LSCs from the spleen of Mll^{PTD/WT}: Flt3^{ITD/ITD} mice, and the cells were co-cultured for 3 days. Flow cytometry plots of the Lin⁻Sca-1⁺c-Kit⁺, Lin⁻Sca-1⁻c-Kit⁺, Lin⁻Sca-1⁺c-Kit⁻, and Lin⁻Sca-1⁻c-Kit⁻cells are shown (n=3). Graphical data for 15M are shown in FIG. 24B. Data are presented as mean±s.d.; P values were calculated with either linear mixed models or one-way ANOVA models. *P<0.05; **P<0.01; ***P<0.001; ****P<0.0001; NS, not significant.

FIGS. 16A-16I show ILC1s inhibit the differentiation of LSCs into myeloid blasts. (A-B) Mouse LSCs labeled with CTV were co-cultured with or without mouse ILC1s for days 1-4, and the percentages (left) and absolute cell numbers (right) of Mac-1⁺ (A) and Gr-1⁺ (B) cells were calculated by flow cytometry (n=3). (C-D) Mouse LSCs labeled with CTV were co-cultured with or without mouse WT, IFN-γ^{-/-}, or TNF-α^{-/-} ILC1s, and percentages of Mac-1⁺ (C) and Gr-1⁺ (D) cells were measured by flow cytometry (n=3). (E) Images of Wright-Giemsa staining of cells (20× magnification, scale bar 100 μm, n=3). (F) Colony-forming cells were counted at each round of plating (n=3); triplicate replicates are shown. (G-H) Mouse LSCs were treated with or without 0.1 ng/ml, 1 ng/ml, or 10 ng/ml recombinant murine IFN-γ, and percentages of Mac-1⁺ (G) and Gr-1⁺ (H) cells were measured by flow cytometry (n=3). (I) Mouse LSCs were sorted and co-cultured with or without sorted ILC1s or treated with IFN-γ for 3 days. LSCs were separated from co-cultured ILC1s using FACS before RNA-seq. Heatmap showing differential expression of RNA from terminal myeloid differentiation and lymphoid differentiation genes (n=3). Data are presented as mean±s.d.; P values were calculated by either one-way ANOVA models or linear mixed models. *P<0.05; **P<0.01; ****P<0.0001; NS, not significant.

FIGS. 17A-17I show ILC1s and IFN-γ improve survival of leukemic mice. (A) Depicts graphical representations of design and procedures for (B-D). On day 0, 3×10⁴ LSCs plus 0.5×10⁶ bone marrow cells isolated from IL-15 transgenic mice (CD45.2) (as support cells) were i.v. co-injected into lethally irradiated (900 cGy) CD45.2 recipient mice. On day 1, the transplanted mice were treated i.v. with 3×10⁴ mouse WT or IFN-γ^{-/-} ILC1s isolated from the liver of corresponding C57BL/6J (CD45.2) mice. (B) Total WBCs were measured at weeks 3 and 6, using the Element HT5 Hematology Analyzer (n=5-7); (C) Images of peripheral blood smears are shown (20× magnification, scale bar 100 μm, n≥3 for each group; Red, red blood cells; Blue, nucleated cells or tumor cells). (D) Survival analysis by Kaplan-Meier and log-rank test are also shown (n=7-9). (E) Depicts graphical representations of design and procedures for (F-I). On day 0, 3×10⁴ LSCs were i.v. co-injected into lethally irradiated (900 cGy) CD45.1 recipient mice along with 0.5×10⁶ bone marrow cells isolated from CD45.1 mice (as support cells). Mice were i.v. injected with 3×10⁴ mouse WT or IFN-γ^{-/-} ILC1s from the liver of C57BL/6J (CD45.2) mice on day 1 or i.p. injected with 0.5 μg/mouse/day recombinant murine IFN-γ for 7 days. (F-H) Statistics on the number of CD45.2⁺ WBCs (F), CD45.2⁺ LSCs (G), and CD45.2⁺ immature blast cells (Mac-1⁺ c-Kit⁺ cells; K) in the blood of recipient mice (n=7). (I) Survival of the mice injected with or without WT ILC1s or IFN-γ^{-/-} ILC or treated with recombinant IFN-γ cytokine. The data were analyzed by Kaplan-Meier survival analysis and log-rank test (n=7). Data are presented as mean±s.d.; P values were calculated by one-way ANOVA models. *P<0.05; **P<0.01; ***P<0.001; NS, not significant.

FIGS. 18A-18K show Normal ILC1s produce significantly more IFN-γ than NK cells when they interact with LSCs via DNAM-1 and IL-7R, which are expressed on ILC1s. (A) Mouse LSCs were co-cultured with or without normal ILC1s or normal NK cells isolated from the liver of normal mice or with or without AML ILC or AML NK cells isolated from the liver of mice with AML for 12 h in the presence of IL-12 (10 ng/ml) plus IL-15 (100 ng/ml). Flow cytometry plots (left) and statistics of IFN-γ⁺ ILC1s or NK cells (right) are shown (n=6-7). (B) The expression of DNAM-1 on ILC1s or NK cells from the liver of normal mice or on AML ILC or AML NK cells from the liver of mice with AML was measured by flow cytometry (n=3). (C) Expression of CD155 and CD112 on mouse LSCs was measured by flow cytometry. (D) Normal liver ILC1s or normal liver NK cells were co-cultured with or without LSCs in the presence or absence of an anti-DNAM-1 neutralizing antibody (10 μg/ml) or isotype IgG control (10 μg/ml) for 12 h along with IL-12 (10 ng/ml) plus IL-15 (100 ng/ml). Flow cytometry plots and statistics of IFN-γ⁺ ILC1s (top) or NK cells (bottom) are shown (n=4). (E) Expression of IL-7R on normal liver ILC or normal liver NK cells. (F) RT-PCR analysis of murine 117 mRNA expression in LSCs, ILC1s, and NK cells. (G) Normal liver ILC1s or normal liver NK cells were co-cultured with or without LSCs in the presence or absence of an anti-IL-7R neutralizing antibody (10 μg/ml) or isotype IgG (10 μg/ml) for 12 h along with IL-12 (10 ng/ml) plus IL-15 (100 ng/ml). Statistics of IFN-γ⁺ ILC1s (n=6). (H) Percentages of IFN-γ⁺ cells in normal liver ILC or normal liver NK cells after treatment with or without IL-7 (100 ng/ml) in the presence of IL-12 (10 ng/ml) plus IL-15 (100 ng/ml) (n=5). (I) Depicts a graphical representation of the overall experimental design for (J and K). CD45.1 mice were i.p. injected with or without IgG control (CTRL; 200 μg/mice), anti-NK1.1 (200 μg/mice), or anti-asialo-GM1 antibody (40 μl/mice) on day 0. Three days later, the mice were i.v. injected with 3×10⁵/mouse LSCs isolated from the spleen of CD45.2 Mll^{PTD/WT}Flt3^{ITD/ITD} mice with AML. The CD45.2⁺ WBCs in the blood were analyzed 6 weeks post LSC transplantation, using the Element HT5 Hematology Analyzer followed by flow cytometry. (J) Flow cytometry plots of the percentages of CD45.1 and CD45.2 cells. (K) Statistical analysis of CD45.2⁺ WBC numbers (n=5). Data are presented as mean±s.d.; P values were calculated by either one-way ANOVA models or Student's t test. *P<0.05; ****P<0.0001; NS, not significant.

FIGS. 19A-19H show IFN-γ derived from ILC1s inhibits LSC differentiation by the JAK-STAT and PI3K-AKT signaling pathways. (A) Mouse LSCs were sorted and co-cultured with or without sorted ILC1s or IFN-γ for 3 days. LSCs were separated from co-cultured ILC1s using FACS before RNA-seq. Heatmap showing RNA differential expression of the top 20 upregulated and downregulated genes (n=3). (B) Hallmark pathway analysis in LSC RNA pools (ILC1-treated vs.

untreated Ctrl). Left panel shows signaling pathways downregulated in LSCs. Right panel shows signaling pathways upregulated in LSCs (n=3). (C) Gene Set Enrichment Analysis (GSEA) plots showing enrichment of selected target genes in LSCs co-cultured with ILC1s. The rank orders (ILC1 vs. Ctrl) of all the genes (n=3) are shown on the X-axis. d-f, Differential expression of RNA from Akt3 (D), Jak2 (E), and Stat1, and Stat2 (F) genes. Results are expressed as means compared with the Ctrl (n=3). (G-H) Mouse LSCs labeled with CTV were treated with or without the indicated JAK and AKT inhibitors for 30 min and then co-cultured with or without WT or IFN-γ^{-/-} ILC1s in the presence of IL-12 (10 ng/ml) and IL-15 (100 ng/ml) for 3 days. Flow cytometry plots (G) and statistics of absolute cell numbers (H) of Lin⁻Sca-1⁺c-Kit⁺, Lin⁻Sca-1⁻c-Kit⁺, Lin⁻Sca-1⁺c-Kit⁻, and Lin⁻Sca-1⁻c-Kit⁻ cells are shown (n=3). Data are presented as mean±s.d.; P values were calculated by one-way ANOVA models. *P<0.05; **P<0.01; ***P<0.001; ****P<0.0001. NS, not significant.

FIGS. 20A-20H show ILC1s are functionally impaired in AML. (A) Mouse LSCs from the spleen of Mll^{PTD/WT}: Flt3^{ITD/ITD} mice with AML were co-cultured with or without ILC1s isolated from the liver of normal mice (Normal ILC1) or Mll^{PTD/WT}: Flt3^{ITD/ITD} mice with AML (AML ILC1) for 3 days. Images are shown (5× magnification, scale bar 200 μm, n=3). (B) Statistics of the percentages of viable of mouse LSCs (n=3). (C) Human LSCs from peripheral blood of patients with AML were co-cultured with or without ILC1s isolated from blood of healthy donors (HD ILC1) or patients with AML (AML ILC1) for 3 days. Images are shown (5× magnification, scale bar 200 μm, n=3). (D) Statistics of the percentages of viable human LSCs (n=6-7). Data from three experiments were pooled. (E) 2,000 LSCs were cultured with or without 1,000 ILC1s from blood of healthy donors or patients with AML for 3 days in the presence of IL-12 (10 ng/ml) and IL-15 (100 ng/ml). Supernatants were collected and subjected to ELISA to determine levels of IFN-γ (n=6-7). Data from three experiments were pooled. (F-H) Mouse LSCs labeled with CTV were co-cultured with or without ILC1s isolated from normal mice or mice with AML in the presence or absence of anti-IFN-γ (10 μg/ml) for 3 days. Flow cytometry plots of the percentages (F), statistics of the percentages (G), and absolute cell numbers (H) of Lin⁻Sea-re-Kit +, Lin⁻Sca-1⁻c-Kit⁺, Lin⁻Sca-1⁺c-Kit⁻, and Lin⁻Sca-1⁻c-Kit⁻cells are shown (n=7). Data from two experiment were pooled. Data are presented as mean±s.d.; P values were calculated by one-way ANOVA. *P<0.05; **P<0.01; ***P<0.001; ****P<0.0001; NS, not significant.

FIGS. 21A-21G show LSCs are present in the liver of mice with AML and ILC1s are also functionally impaired in mice with AML. (A) 0.2×10⁶ LSKs isolated from the liver of normal mice or Mll^{PTD/WT}: Flt3^{ITD/ITD} mice with AML were i.v. injected into immunodeficient Rag2^{-/-}γc^{-/-} mice. The survival of those mice was analyzed by the Kaplan-Meier method and log-rank test (n=3). (B) LSCs were isolated from the spleen of Mll^{PTD/WT}: Flt3^{ITD/ITD} mice with AML (CD45.2) and then transplanted into lethally irradiated mice (CD45.1). The percentages of LSCs in the liver of the CD45.1 mice were analyzed 9 weeks after LSC transplantation using flow cytometry. (C) Gating strategy for flow cytometry analysis of the mouse ILC1s isolated from livers. The mouse ILC1s were defined as CD3⁻CD19⁻NK1.1⁺NKp46⁺CD49b⁻CD49a⁺. (D-F) 2×10⁶ C1498 cells were i.v.

injected into C57BL/6J mice. Twenty-one days later, the production of IFN-γ and TNF-α by ILC1s from the liver (D), bone marrow (E), and spleen (F) of those normal mice or mice with AML are shown (n=5). (G) GSEA plot shows the relative abundance of genes involved in the TNF-α-NF-κB signaling pathways in liver ILC isolated from mice with AML or normal mice (n=3). All data (D-F) are shown as mean±s.d.; P values were calculated by Student's t test. *P<0.05; **P<0.01; NS, not significant.

FIGS. 22A-22F show Gating strategy for flow cytometry analysis. (A-B) Purity of LSCs (A) and ILC1s (B) after sorting. (C) Gating strategy for flow cytometric analysis of apoptosis of LSCs co-cultured with or without ILC1s, using 7-AAD. CTV: CellTrace™ Violet. (D) Gating strategy for flow cytometry analysis of apoptosis of LSCs co-cultured with ILC1s using the Violet Live Cell Caspase Probe. (E) Gating strategy for flow cytometry analysis of human ILC1s isolated from peripheral blood. Lineage markers: CD3, CD4, CD8, CD14, CD15, CD16, CD19, CD20, CD33, CD34, CD203c, and FceRI. Human ILC1s were defined as

Lin⁻CD56⁻CD127⁺c-Kit⁻CRTH2⁻. (F) Gating strategy for flow cytometry analysis of human LSCs. Lineage markers: CD2, CD3, CD4, CD8, CD14, CD16, CD19, Mac-1, CD56, and CD235a. Human LSCs were defined as Lin⁻CD45^{dim}CD34⁺CD38⁻.

FIGS. 23A-23C show IFN-γ—but not TNF-α—induces apoptosis of LSCs. (A) 5,000-10,000 murine liver ILC1s were sorted and transferred into the top wells of a 96-well Transwell plate. The bottom chambers of the plate were loaded with 10,000-20,000 LSCs from the spleens of Mll^{PTD/WT}: Flt3^{ITD/ITD} mice with AML. The cells were then co-cultured for 3 days. The percentages of LSCs that were apoptotic were measured by flow cytometry (n=3). Data are shown as mean±s.d.; P values were calculated by one-way ANOVA models. NS, not significant. (B-C) LSCs from the spleen of Mll^{PTD/WT}: Flt3^{ITD/ITD} mice with AML were treated with or without the indicated doses of IFN-γ or TNF-α for 3 days. Representative images (top, 5× magnification, scale bar 200 μm) and flow cytometry plots (bottom) of the percentages of apoptotic cells in LSCs are shown.

FIGS. 24A-24B show ILC1s and IFN-γ inhibit the differentiation of LSCs into leukemia progenitor cells and promote their differentiation into non-leukemic LS⁺K⁻ cells. (A) LSCs from the spleen of Mll^{PTD/WT}: Flt3^{ITD/ITD} mice with AML were co-cultured with or without 0.1 ng/ml, 1 ng/ml, or 10 ng/ml recombinant murine IFN-γ. Then, the percentages of Lin⁻Sca-1⁺c-Kit⁺, Lin⁻Sca-1⁻c-Kit⁺, and Lin⁻Sca-1⁺c-Kit⁻ cells were measured by flow cytometry (n=4). (B) ILC1s from normal mouse liver were sorted and transferred into the top well of a 96-well Transwell plate. The bottom chamber of the plate were loaded with LSCs from the spleen of Mll^{PTD/WT}: Flt3^{ITD/ITD} mice with AML, and co-incubated for 3 days (n=3). Then, the percentages of Lin⁻Sca-1⁺Lin⁻Sca-1⁻c-Kit⁺, and Lin⁻Sca-1⁺c-Kit⁻ cells were measured by flow cytometry. All data shown as mean±s.d.; P values were calculated by one-way ANOVA. *P<0.05; **P<0.01; ***P<0.001; ****P<0.0001; NS, not significant.

FIGS. 25A-25D show ILC1s and IFN-γ do not affect the differentiation of leukemia progenitor cells into myeloid blasts. (A-B) Mouse LSCs labeled with CTV were co-cultured with or without mouse ILC1s in the presence or absence of anti-IFN-γ or anti-TNF-α antibody. Statistics of absolute cell numbers of Mac-1⁺ (A) and Gr-1⁺ (B) cells are shown (n=3). (C) Leukemia progenitor cells were sorted from the spleen of Mll^{PTD/WT}: Flt3^{ITD/ITD} mice with AML and co-cultured with or without WT ILC1s, IFN-γ^{-/-} ILC1s, or IFN-γ. Flow cytometry plots (top) and statistics of the percentages (bottom) of Mac-1⁺ and Gr-1⁺ cells are shown (n=3). (D) A graphic representation of a working model showing how ILC1s and their secreted IFN-γ regulate differentiation of LSCs. All data are shown as mean±s.d.; P values were calculated by one-way ANOVA models. *P<0.05; **P<0.01; ***P<0.001; ****P<0.0001; NS, not significant.

FIGS. 26A-26J show ILC1s do not induce apoptosis of normal HSCs or impair their differentiation. (A) Mouse HSCs (Lin⁻Sca-1⁺c-Kit⁺ cells) from bone marrow of normal mice were co-cultured with or without mouse ILC1s for 3 days. Images (left) and statistics of the percentages (right) of apoptotic cells are shown (5× magnification, scale bar 200 μm, n=5). (B) Human HSCs (Lin⁻CD34⁺ cells) from blood of healthy donors were co-cultured with or without human ILC1s for 3 days. Images (left) and statistics of the percentages (right) of apoptotic cells are shown (n=4). (C-E) Mouse HSCs labeled with CTV were co-cultured with or without mouse ILC1s, and flow cytometry plots (C), statistics of absolute cell numbers (D), and percentages (E) of Lin⁻Sca-1⁺c-Kit⁺ and Lin⁻Sca-1⁻c-Kit⁺ cells are shown (n=4). (F) Flow cytometry plots (left) and statistics (right) of absolute cell numbers of Mac-1⁺Gr-1⁺ cells (n=4). (G) Depicts a graphical representation of experimental scheme for (H-J). Mouse HSCs (3×10⁴) isolated from bone marrow of CD45.2 normal mice were i.v. injected into lethally irradiated CD45.1 mice. One day later, 3×10⁴ ILC1s isolated from the liver of normal mice were i.v. injected into CD45.1 recipient mice that had already been injected with HSCs. Three weeks later, donor hematopoietic and progenitor cells, myeloid cell subsets, and WBCs derived from the CD45.2 mice were analyzed by flow cytometry. (H) The absolute cell numbers of donor LSKs, myeloid progenitor cells (L⁻S⁻K⁺, Lin⁻Sca-1⁻c-Kit⁺ cells), early lymphoid-committed precursors (L⁻S⁺K⁻, Lin⁻Sca-1⁻c-Kit⁻ cells), short-term hematopoietic stem cells (STHSC, Lin⁻Sca-1⁺c-Kit⁺Flt3⁻CD150⁻CD48⁻ cells), long-term hematopoietic stem cells (LTHSC, Lin⁻Sca-1⁺c-Kit⁺Flt3⁻CD150⁺CD48⁻cells), multipotent progenitors 1 and 2 (MPP1, Lin⁻Sca-1⁺c-Kit⁺Flt3⁻CD150⁺CD48⁺cells; MPP2, Lin⁻Sca-1⁺c-Kit⁺Flt3⁻CD150⁻CD48⁺ cells), Mac-1⁺Gr-1⁺ cell subsets, and WBCs derived from CD45.2 mice were determined by the trypan blue exclusion assay together with flow cytometry. (I) Flow cytometry plots (left) and statistics of absolute cell numbers (right) of Mac-1⁺Gr-1⁺ cells derived from CD45.2 donor mice, determined by the trypan blue exclusion assay together with flow cytometry. (J) Absolute cell numbers of WBCs determined by cell counting with the Element HT5 Hematology Analyzer and subsequent flow cytometry. All data are shown as mean±s.d.; P values were calculated by Student's t-test or one-way ANOVA. NS, not significant.

FIGS. 27A-27C show ILC1s reduce the leukemia burden of mice. (A) 3×10⁴ LSCs were i.v. co-injected into lethally irradiated (900 cGy) CD45.2 recipient mice on day 0 along with 0.5×10⁶ bone marrow cells isolated from IL-15 transgenic mice (CD45.2) as support cells. On day 1, the mice were i.v. injected with 3×10⁴ WT ILC1s from the liver of C57BL/6J (CD45.2) mice or i.p. injected daily with recombinant murine IFN-γ (0.5 μg/mouse/day). Statistics of the numbers of total WBCs at week 5 are shown (n=4-6). All absolute cell numbers of WBCs were determined by cell counting with the Element HT5 Hematology Analyzer. (B) Flow cytometry plots of the percentages of CD45.1⁺ and CD45.2⁺ cells. (C) 3×10⁴ LSCs were i.v. co-injected into lethally irradiated (900 cGy) CD45.1 recipient mice on day 0 along with 0.5×10⁶ bone marrow cells isolated from CD45.1 mice (as support cells). Mice were i.v. injected with 3×10⁴ WT mouse ILC or IFN-γ^{-/-} ILC1s from the liver of C57BL/6J (CD45.2) mice on day 1 or i.p. injected daily with recombinant murine IFN-γ (0.5 μg/mouse/day). Statistics of the number of total WBCs at week 3 are shown (n=7). All absolute cell numbers of WBCs were determined by cell counting with the Element HT5 Hematology Analyzer followed by flow cytometry. All data are shown as mean±s.d.; P values were calculated by one-way ANOVA. *P<0.05; ****P<0.0001; NS, not significant.

FIGS. 28A-28G show Involvement of IL-7-IL-7R signaling in IFN-γ production by liver ILC1s rather than NK cells; induction of LSC apoptosis via IFN-γ from ILC1s but not NK cells; and optimizing the depletion of ILC1s and NK cells (double depletion) or NK cells only with anti-NK1.1 and anti-asialo GM1 antibody, respectively. (A) Normal mouse liver ILC1s or NK cells were co-cultured with or without LSCs in the presence or absence of anti-IL-7R neutralizing antibody (10 μg/ml) or isotype IgG control (10 μg/ml) for 12 h along with IL-12 (10 ng/ml) plus IL-15 (100 ng/ml). Flow cytometry plots of IFN-γ production by ILC1s (n=6). (B) Flow cytometry plots of IFN-γ production in normal liver ILC or normal liver NK cells after treatment with or without IL-7 (100 ng/ml) in the presence of IL-12 (10 ng/ml) plus IL-15 (100 ng/ml) (n=5). (C-D) Mouse LSCs were co-cultured with or without mouse ILC1s or NK cells for 3 days in the presence or absence of mouse anti-IFN-γ (10 μg/ml). Images (C; 5× magnification, scale bar 200 μm) and statistics of absolute cell numbers (D) are shown (n=3). (E-F) Flow cytometry plots (E) and statistics of the percentages of apoptotic LSCs (F) (n=3). (G) To deplete ILC1s or NK cells, WT mice were i.p. injected with an IgG control (CTRL, 200 μg/mouse), anti-NK1.1 (200 μg/mouse), or anti-asialo-GM1 (40 μl/mouse) antibody. Three days later, the percentages of NK cells (Lin⁻NK1.1⁺NKp46⁺CD49b⁺) and ILC1s (Lin⁻NK1.1⁺NKp46⁺CD49a⁺) in the liver of WT mice were measured by flow cytometry. Data (D and F) are shown as mean±s.d.; P values were calculated by one-way ANOVA models. *P<0.05; **P<0.01; NS, not significant.

FIGS. 29A-29G show RNA-seq identified upregulated and downregulated genes and signaling pathways in LSCs treated with ILC1s or IFN-γ. (A) Depicts a graphical representation of the experimental design for RNA sequencing (RNA-Seq). Mouse LSCs were sorted and treated with or without sorted ILC1s or IFN-γ for 3 days. LSCs were resorted from co-cultured ILC1s or IFN-γ using FACS before RNA-Seq. (B) Purity of LSCs (left) and ILC1s (right) after cell sorting. (C) Heat map showing differential expression of RNA of 627 genes (n=3). (D-F) Volcano plots showing significantly differentially expressed genes in RNA pools from AML LSCs treated with ILC vs. Ctrl (untreated) (D), IFN-γ vs. Ctrl (E), and IFN-γ vs. ILC (F) (n=3). (G) Hallmark pathway analysis in LSC RNA pools (IFN-γ vs. Ctrl). The left panel shows signaling pathways downregulated in LSCs. The right panel shows signaling pathways upregulated in LSCs (n=3). Genes with an FDR-adjusted P-value<0.05 and a fold change (FC) >1.5 or <0.7 were considered as significant upregulated and downregulated genes, respectively.

FIGS. 30A-30H show ILC1s or IFN-γ inhibit the differentiation of LSCs via the JAK-STAT and AKT signaling pathways. (A) GSEA plots show enrichment of the indicated target genes in LSCs co-cultured with ILC1s. The X-axis shows the rank orders (ILC1s vs. Ctrl) of all the genes. (B) GSEA plots show enrichment of the indicated target genes in LSCs treated with IFN-γ. The X-axis shows the rank orders (IFN-γ vs. Ctrl) of all the genes. (C-D) Heat maps showing differential expression of RNAs of genes downstream of IFN-γ. (E-H) Mouse LSCs labeled with CTV were treated with or without the indicated JAK and AKT inhibitors for 30 min and then co-cultured with or without WT or IFN-γ^{-/-} ILC1s in the presence of IL-12 (10 ng/ml) and IL-15 (100 ng/ml) for 3 days. Statistics of the percentages of Lin Sca-1⁺c-Kit⁺, Lin⁻Sca-1⁻Lin⁻Sca-1⁺c-Kit⁻, and Lin⁻Sca-1⁻c-Kit⁻cells. Genes with an FDR-adjusted P-value <0.05 and a fold change (FC) >1.5 or <0.7 were considered to be significantly upregulated or downregulated. Data (E-H) are shown as mean±s.d.; P values were calculated by one-way ANOVA models. *P<0.05; **P<0.01; ***P<0.001; ****P<0.0001; NS, not significant.

### DETAILED DESCRIPTION

Innate lymphoid cells (ILCs) are a heterogeneous population of non-B and non-T lymphocytes that originate from the common lymphoid progenitor (CLP) and lack antigen-specific receptors. ILCs can be classified into three groups based on the unique cytokines that they produce and the transcription factor signatures that drive their differentiation: group 1 ILCs (comprised of natural killer [NK] cells and type I innate lymphoid cells [ILC1s]), group 2 ILCs (ILC2s), and group 3 ILCs (ILC3s)⁶. ILC1s, which usually reside in the liver, produce the cytokines IFN-γ, granulocyte macrophage-colony stimulating factor (GM-CSF), TNF-α, and TNF-related apoptosis inducing ligand (TRAIL), and express T-BET but lack expression of EOMES. ILC2s produce the cytokines IL-4, IL-5, and IL-13 and express the transcription factor GATA3. ILC3s produce the cytokines IL-22 and IL-17A and express the retinoic acid-related orphan receptor γt (RORγt) transcription factor^{7,8}. Recent studies reported that ILCs, especially ILC2s^{9,10} and ILC3s¹¹⁻¹³, play a key role in antivirus or antimicrobial immune response, tumor surveillance, and tumorigenesis. However, no available studies have elucidated the interaction of ILC1s with tumor cells, in particular cancer stem-like cells or LSCs, and the relevance of this interaction to anti-tumor response in patients with a cancer such as AML.

In the present study, the inventors discovered that ILC1s target LSCs in AML. They discovered that ILC1s isolated from normal mice or healthy humans induce LSC apoptosis, mainly via secretion of IFN-γ, while in AML, these multifaceted functions of ILCs were impaired. They performed a series of functional and mechanistic studies to characterize the important roles that ILC1s play in inhibiting LSC differentiation into leukemia progenitor cells, blocking differentiation into terminal myeloid blasts, and as a result, suppressing leukemogenesis.

This work demonstrated that ILC1s isolated from normal mice or healthy humans induced LSC apoptosis. Further, normal ILC1s target LSCs to suppress leukemogenesis by preventing their differentiation into leukemia progenitor, thus blocking their differentiation into terminal myeloid blasts. Without being bound by theory, these effects occurred via the production of interferon-γ by ILC1s. Moreover, ILC produced more IFN-γ than NK cells through the receptors DNAM-1 and IL-7R interacting with LSCs. Because these functions are impaired in AML, ILC1s can no longer effectively target LSCs, which can then differentiate into leukemia cells. Collectively, these data define an essential protective role for ILC1s in AML: inducing apoptosis and targeting differentiation of LSCs.

The methods described herein include methods for the treatment of disorders associated with cancer or leukemia. In some embodiments, the disorder is a cancer or leukemia (e.g., Acute lymphocytic leukemia (ALL), Acute myeloid leukemia (AML), Chronic lymphocytic leukemia (CLL), Chronic myeloid leukemia (CML), Hairy cell leukemia (HCL), or Myelodysplastic syndromes (MDS)). Generally, the methods include administering a therapeutically effective amount of ILC1s as described herein, to a subject who is in need of, or who has been determined to be in need of, such treatment.

As used in this context, to “treat” means to ameliorate at least one symptom of the disorder associated with cancer or leukemia. ILC1 treatment results in elimination, killing, or reducing cancer or leukemia cells; thus, ILC1 treatment can result in a reduction in or prevention of relapse of the cancer or leukemia and a prolonged survival or prolonged relapse-free survival. Administration of a therapeutically effective amount of a composition described herein for the treatment of a condition associated with cancer or leukemia will result in decreased cancer or leukemia cells, increased IFN-γ (e.g., in the tumor micro environment (TME), and/or prolong survival.

### EXAMPLES

The invention is further described in the following examples, which do not limit the scope of the invention described in the claims.

### Materials and Methods

The following materials and methods were used in the Examples set forth herein.

### Human Samples

Peripheral blood (PB) and bone morrow samples from healthy and AML individuals were obtained from donors at the City of Hope National Medical Center (COHNMC). Mononuclear cells were isolated using Ficoll separation. Lin⁻CD34⁺CD38⁻ cells were sorted by Aria Fusion III. Lin⁻CD45^{dim}CD34⁺CD38⁻ cells were sorted using a BD FACSAria™ Fusion (BD Biosciences). All patients with AML and healthy donors signed an informed consent form. Sample acquisition was approved by the Institutional Review Boards at the COHNMC.

### Mouse Studies

C57BL/6J, Rag2^{-/-}γc^{-/-}, Mll^{PTD/WT}/Flt3^{ITD/ITD}, IL-15 transgenic, IFN-γ^{-/-} and TNF-α^{-/-} mice were maintained by the Animal Resource Center of City of Hope. 8 to 12-week-old Rag2^{-/-} 665 c^{-/-} or C57BL/6J mice of both sexes were used as recipients for AML cell transplantation. Mll^{PTD/WT}/Flt3^{ITD/ITD} mice of both sexes were used as donor mice. Mouse care and experimental procedures were performed in accordance with federal guidelines and protocols approved by the Institutional Animal Care and Use Committee at the City of Hope.

C57BL/6J (B6, CD45.2), Rag2^{-/-} γc^{-/-}, TNF-α^{-/-} and CD45.1 (B6.SJL-Ptprc^{a}Pepc^{b}/BoyJ) were purchased from the Jackson Laboratory. Mll^{PTD/WT}: Flt3^{ITD/ITD} mice 24 and IL-15 transgenic mice³⁴ on the B6 background were generated as described previously. All mice were maintained by the Animal Resource Center of COH. Six- to twelve-week-old CD45.2 and CD45.1 mice of both sexes were used as recipients for AML cell transplantation; Mll^{PTD/WT}: Flt3^{ITD/ITD} mice with AML of both sexes were used as donor mice. Mouse care and experimental procedures were performed in accordance with federal guidelines and protocols approved by the Institutional Animal Care and Use Committee at City of Hope.

### Cells and Cell Culture

Human LSCs were cultured in StemSpan™ SFEM II (Stem cell, USA) with penicillin (100 U/mL) and streptomycin (100 mg/mL). Stem cell factor (SCF, 20 ng/ml), thrombopoietin (TPO, 20 ng/ml), erythropoietin (EPO, 20 ng/ml), Flt3-L (20 ng/ml), IL-3 (10 ng/ml), and IL-6 (10 ng/ml). Mouse LSCs were cultured in IMDM with 10% FBS, penicillin (100 U/mL), streptomycin (100 mg/mL), SCF (20 ng/ml), TPO (20 ng/ml), Flt3-L (20 ng/ml), IL-3 (10 ng/ml), and IL-6 (10 ng/ml). Human and mouse ILC1s or NK cells were cultured in RPMI 1640 with 10% FBS, penicillin (100 U/mL), streptomycin (100 mg/mL), IL-12 (10 ng/ml), and IL-15 (100 ng/ml). Mouse AML cell lines (C1498) were cultured in RPMI 1640 with 10% FBS, penicillin (100

U/mL) and streptomycin (100 mg/mL). Cultures were incubated at 37° C. in a humidified atmosphere of 5% CO2. All cell lines are from American Type Culture Collection (ATCC). All cytokines are from PeproTech.

### Flow Cytometry

ILC1s from human peripheral blood were identified by a surface stain including a live/dead cell viability cell staining kit (Invitrogen) and the following monoclonal antibodies: lineage (FITC-conjugated anti-CD3, anti-CD4, anti-CD8, anti-CD14, anti-CD15, anti-CD16, anti-CD19, anti-CD20, anti-CD33, anti-CD34, anti-CD203c, anti-FceRI), CD56 (FITC, AF700 or BV421 conjugated anti-CD56), CD127 (APC-conjugated anti-CD127), CRTH2 (PE-Cy7-conjugated anti-CRTH2), and c-Kit (PE-conjugated anti-c-Kit). ILC from mice were identified by a surface stain and the following monoclonal antibodies: lineage (PE-Cy7-conjugated anti-CD3 and anti-CD19), NK1.1 (BV510-conjugated anti-NK1.1), NKp46 (BV421, FITC or AF647-conjugated anti-NKp46), CD49b (BUV395 or PE-conjugated anti-CD49b), and CD49a (BV711-conjugated anti-CD49a). Human LSCs were identified by lineage (FITC-conjugate anti-CD2, anti-CD3, anti-CD4, anti-CD8, anti-CD14, anti-CD19, anti-CD20, anti-Mac-1, anti-CD56, and anti-CD235a), CD45 (BV510-conjugated anti-CD45), CD34 (BV510-conjugate anti-CD34), and CD38 (BV605 conjugated anti-CD38). Mouse LSCs were identified by lineage (PE-Cy7 conjugated anti-CD3, anti-CD19, anti-B220, anti-Ly6G/C, anti-Mac-1, anti-CD11b, and anti-Ter119), Sca-1 (PE-CF594 or BV510-conjugate anti-Sca-1), and c-Kit (BV711-conjugated anti-c-Kit). Mouse long-term hematopoietic stem cells (LTHSCs), short-term hematopoietic stem cells (STHSCs), and multipotent progenitors (MPP) 1 and 2 were identified by lineage, Sca-1, c-Kit, Flt3 (APC-conjugated anti-Flt3), CD150 (PE-conjugated anti-CD150), and CD48 (FITC or BV786-conjugated anti-CD48). The expression of CD155 and CD112 on mouse LSCs was identified by APC-conjugated anti-CD155 and BV786-conjugated anti-CD112, respectively. The expression of DNAM-1 and IL-7R on mouse ILC1s was identified by BV421-conjugated anti-DNAM-1 and PerCP-Cy5.5-conjugated anti-IL-7R, respectively. The expression of CD45.1 and CD45.2 were identified by BV605-conjugated-anti-CD45.1 and APC/Fire™ 750- or FITC- conjugated-anti-CD45.2, respectively. Human ILC1s were gated by Lin⁻CD56⁻CD127⁺CRTH2⁻c-Kit⁻. Mouse ILC1s were gated by Lin⁻NK1.1⁺NKp46⁺CD49b⁻CD49a⁺. Mouse NK cells were gated by Lin⁻NK1.1⁺NKp46⁺CD49b^{+CD}49a⁻. Human LSCs were gated by Lin⁻CD45^{dim}CD34⁺CD38⁻. Mouse LSCs were gated by Lin⁻Sca-1⁺c-Kit⁺. Mouse LTHSCs were gated by Lin⁻Sca-1⁺c-Kit⁺Flt3⁻CD150⁺CD48⁻. Mouse STHSCs were gated by Lin⁻Sca-1⁺c-Kit⁺Flt3⁻CD150⁻CD48⁻. Mouse MPPls were gated by Lin⁻Sca-1⁺c-Kit⁺Flt3⁻CD150⁻CD48⁺. Mouse MPP2s were gated by Lin⁻Sca-1⁺c-Kit^{+Flt}3⁻CD150⁺CD48⁺. Myeloid cells were gated by Mac-1⁺Gr-1⁺. To examine intracellular cytokine production, mouse ILC1s or NK cells co-cultured with or without LSCs were stimulated by IL-12 (10 ng/ml) and IL-15 (100 ng/ml) or IL-7 (100 ng/ml) for 4 h or 12 h in the presence of BD GolgiPlug™. Human ILC were gated by Lin⁻CD56⁻CD127⁺CRTH2⁻c-Kit⁻. Mouse ILC1s were gated by Lin⁻ NK1.1⁺NKp46⁺CD49b⁻CD49a⁺. Human LSCs were gated by Lin⁻CD34⁺CD38⁻. Mouse LSCs were gated by Lin⁻Scal-1⁺c-Kit⁺. Intracellular staining for TNF-α or IFN-γ was performed using a Fix/Perm kit (eBiosciences), followed by staining with an AF700-conjugated anti-TNF-α antibody or a BV786-conjugated anti-IFN-γ antibody, respectively. All analyses were performed on a Fortessa X-20 flow cytometer (BD Biosciences) and sorting was performed using Aria Fusion III instruments (BD Biosciences) or a BD FACSAria™ Fusion (BD Biosciences).

### Isolation of ILC1s or NK Cells

To isolate ILC or NK cells from mouse liver, we washed harvested liver and pressed it through a 100 μm mesh to make single cells, which were washed once with PBS. The cells were re-suspended in 40% Percoll (Sigma-Aldrich) and then gently overlaid on 70% Percoll, followed by centrifugation according to the manufacturer's instructions. Mononuclear cells (MNCs) were collected from the interphase and washed twice with PBS. The washed MNCs were stained with anti-CD3, anti-CD19, anti-NK1.1, anti-NKp46, anti-CD49b, and anti-CD49a antibodies. Thirty minutes later, the cells were washed 3 times and then sorted using BD FACSAria™ Fusion.

To isolate ILC1s from human peripheral blood, we diluted blood cone samples 1:1 with phosphate-buffered saline (PBS). We layered the blood on the top of Ficoll-Paque (GE Healthcare), and centrifuged it according to the manufacturer's instructions. The mononuclear cell fraction was aspirated and washed with PBS, and then the red blood cells were lysed. The mononuclear cells were stained with lineage (anti-CD3, anti-CD4, anti-CD8, anti-CD14, anti-CD15, anti-CD16, anti-CD19, anti-CD20, anti-CD33, anti-CD34, anti-CD203c, anti-FceRI, and anti-CD56), anti-CD127, anti-CRTH2, and anti-c-Kit antibodies. Thirty minutes later, the cells were washed 3 times and then sorted using BD FACSAria™ Fusion.

### LSCs and ILC1s In Vitro Co-Culture Assay

A total of 2,000 LSCs from AML patients labeled with CTV were co-cultured with different numbers of human ILC1s supplemented with human IL-12 (10 ng/ml) and IL-15 (100 ng/ml). After 3 days of co-culture, cells were harvested and analyzed by flow cytometry. 7-AAD was used to identify dead cells. For mouse LSCs co-culture assay, 2,000 LSCs from Mll^{PTD/WT}/Flt3^{ITD/ITD} mice labeled with CTV were co-cultured with different numbers of mouse ILC1s supplemented with mouse IL-12 (10 ng/ml) and IL-15 (100 ng/ml). After 3 days of co-culture, cells were harvested and analyzed by flow cytometry. 7-AAD was used to identify the dead cells. For the co-culture of LSCs and ILC1s using the transwell co-culture system, 2,000 human or mouse LSCs were seeded in the lower chamber while different numbers of human or mouse ILC1s were seeded in the upper chamber. After 3 days, cells were harvested and analyzed by flow cytometry. 7-AAD was used to identify the dead cells. For co-culture assay with cytokines and antibodies, 2,000 human or mouse LSCs were co-cultured with different doses of human or mouse TNF-α, IFN-γ, anti-TNF-α (10 μg/ml) Ab, or anti-IFN-γ Ab (10 μg/ml). Three days after the co-culture, cells were harvested and analyzed by flow cytometry. 7-AAD was used to identify the dead cells.

For mouse LSC co-culture assays, LSCs from Mll^{PTD/WT}: Flt3^{ITD/ITD} mice with AML were labeled with 5 mM CellTrace Violet (CTV, Thermo Fisher Scientific, USA) and co-cultured in the presence of mouse IL-12 (10 ng/ml) and IL-15 (100 ng/ml) with various numbers of ILC1s or NK cells isolated from liver of normal mice or mice with AML. For human LSC co-culture assays, LSCs from patients with AML were labeled with 5 mM CTV and co-cultured in the presence of human IL-12 (10 ng/ml) and IL-15 (100 ng/ml) with various numbers of ILC1s isolated from peripheral blood of healthy donors or patients with AML. For co-culture of LSCs and ILC1s in the Transwell co-culture system, LSCs were seeded in the lower chamber of a 96-well Transwell plate, while varying numbers of mouse ILC1s were seeded in the upper chamber. For co-culture assays with cytokines and antibodies, mouse or human LSCs were co-cultured with various doses of mouse TNF-α (0.25 μg/ml, 0.5 μg/ml, 0.75 μg/ml, and 1 μg/ml), mouse IFN-γ (0.1 ng/ml, 1 ng/ml, 10 ng/ml, 0.25 μg/ml, 0.5 μg/ml, 0.75 μg/ml, and 1 μg/ml), anti-TNF-α (10 μg/ml) antibody, or anti-IFN-γ antibody (10 μg/ml). For all co-culture assays, cells were harvested after 3 days and analyzed using flow cytometry. 7-amino-actinomycin D (7-AAD, BD Biosciences) was used to identify dead cells following the manufacturers' instructions. Cell images were taken by microscope (ZEISS).

For the LSC differentiation assay, 1,000 LSCs were isolated from Mll^{PTD/WT/Flt}3^{ITD/ITD} mice and then were co-cultured with or without 500 ILC1s isolated from normal mouse livers for 1 to 4 days. LSCs were isolated from Mll^{PTD/WT}: Flt3^{ITD/ITD} mice with AML and co-cultured with or without ILC1s isolated from liver of normal mice or Mll^{PTD/WT}: Flt3^{ITD/ITD} mice with AML for 1 to 4 days in the presence or absence of anti-TNF-α (10 μg/ml) antibody or anti-IFN-γ antibody (10 μg/ml)Cells were harvested and analyzed by flow cytometry.

### In Vivo LSC Transplantation Assay

In all the transplantation experiments, recipient mice were placed on sulfatrim-based food (5053/.025% Tri/.1242% Sulf ½ IRR; Catalogue number: 5W8F; TestDiet, Richmond, IN) post-transplantation to avoid any infection/toxicity-associated with irradiation. 1×10⁶ support bone marrow cells depleted of NK1.1⁺NKp46⁺ cells from IL-15 transgenic mice were transplanted by tail vein injection with 30,000 LSCs that were obtained from Mll^{PTD/WT}/Flt3^{ITD/ITD} transgenic mice into lethally (900 cGy, 96 cGy/min, γ-rays) irradiated 6- to 10-week-old B6.SJL (Ly5.1) or C57BL/6 (CD45.2) recipient mice. Next, WT or IFN-γ ILC1s, which were purified from C57BL/6 mice, were injected by tail vein injection (30,000 cells/mouse) into these mice. In some experiments, 0.5 μg per mice animal-free recombinant murine IFN-γ were intraperitoneally injected into recipient mice for 10 days. The number of white blood cells (WBCs), neutrophils and monocytes were monitored every 3 weeks. In Rag2^{-/-} γc^{-/-} mice experiments, 30,000 LSCs were transplanted into 200 cGy irradiated 6- to 10-week-old Rag2^{-/-} γc^{-/-} mice, followed by multiple injection of ILC1s. The number of WBCs, neutrophils and monocytes were monitored every 3 weeks. Leukemic mice were euthanized by CO2 inhalation when they showed signs of systemic illness.

0.5×10⁶ bone marrow cells from CD45.1 WT mice or bone marrow cells isolated from IL-15 transgenic mice (CD45.2) depleted of NK1.1⁺ NKp46⁺ cells were transplanted by i.v injection with 3×10⁴ LSCs obtained from Mll^{PTD/WT}: Flt3^{ITD/ITD} mice with AML into lethally (900 cGy, 96 cGy/min, 665 -rays) irradiated 6- to 12-week-old C57BL/6J (CD45.1) recipient mice. Next, WT or IFN-γ^{-/-} ILC1s (CD45.2), which were purified from WT or IFN-γ^{-/-} C57BL/6J mice, were injected via i.v. into recipient mice (3×10⁴ cells/mouse). In some experiments, animal-free recombinant murine IFN-γ (0.5 μg/mouse) was i.p. injected into recipient mice daily for 7 days. For all transplantations, the numbers of WBCs, LSCs, or immature blast cells in peripheral blood were counted at the indicated times using Element HT5 hematology analyzer and flow cytometry. (Peripheral blood was also collected for making blood smear slides). Blood smear slides were stained with Wright-Giemsa (Polysciences). Leukemic mice were euthanized using CO2 inhalation when they showed signs of systemic illness.

In Vivo HSC Transplantation Assay In all transplantation experiments, recipient mice were fed with sulfatrim-based food (Catalogue number: 5W8F; TestDiet, Richmond, IN) post-transplantation to avoid any infection/toxicity associated with irradiation. 3×10⁴ HSCs were isolated from bone marrow cells of normal CD45.2 mice and i.v. co-injected with 5×10⁵ CD45.1 bone marrow cells (as support cells) into lethally irradiated (900 cGy) 6- to 12-week-old C57BL/6J (CD45.1) recipient mice. One day later, 3×10⁴ ILC1s (CD45.2) isolated from the liver of normal mice were i.v. injected into these recipient mice. The LSKs, Lin⁻Sca-1⁻c-Kit⁺ cells, Lin⁻Sca-1⁺c-Kit⁻ cells, STHSCs, LTHSCs, MPP1, MPP2, Mac-1⁺Gr-1⁺ cells, and WBCs derived from donor mice were analyzed 3 weeks post HSC transplantation using Element HT5 hematology analyzer (Heska, USA) and flow cytometry (BD Biosciences).

### Caspase3/7 Activity Assay

ILC1s were co-cultured with LSCs at a ratio of 1:1 or 1:2 for 6 h. Next, 100 μl of Caspase-Glo 3/7 reagent was added to each well. Plates were then shaken at 300 rpm for 1 min, incubated for 60 min at room temperature, and then read on a luminometer (Promega, Glomax). Background luminescence was determined with 100 μl of culture medium without cells and subtracted before fold changes were calculated.

### In Vitro Stimulation of ILC1s and NK Cells

Mouse ILC1s or NK cells were sorted from the liver of normal mice or mice with AML and then were co-cultured with or without LSCs for 12 h in the presence of IL-12 (10 ng/ml) plus IL-15 (100 ng/ml).

For stimulation by anti-DNAM-1 or anti-IL-7R neutralizing antibody or isotype IgG control, mouse ILC1s or NK cells were sorted from the liver of normal mice and co-cultured with or without an anti-DNAM-1 (10 μg/ml) or anti-IL-7R neutralizing antibody (10 μg/ml) at 5% CO2 and 37° C. in RPMI-1640 culture medium supplemented with 10% FBS, penicillin (100 U/mL), streptomycin (100 mg/mL), IL-12 (10 ng/ml), and IL-15 (100 ng/ml). Thirty minutes later, LSCs were added at an equal ratio to some of the cultures of the ILC1s or NK cells and then co-cultured for 12 h. For stimulation with recombinant mouse IL-7, mouse ILC1s or NK cells were sorted from the liver of normal mice and then were treated with or without recombinant mouse IL-7 (100 ng/ml) for 12 h at 5% CO2 and 37° C. in RPMI-1640 culture medium supplemented with 10% FBS, penicillin (100 U/mL), streptomycin (100 mg/mL), IL-12 (10 ng/ml), and IL-15 (100 ng/ml).

For all of the above stimulation assays, BD GolgiPlug™ was added to the cultures 4 h before cells were collected. Then cells were harvested, washed, and stained for surface molecules and intracellular IFN-γ. Percentages of IFN-γ⁺ ILC1s or NK cells were calculated by flow cytometry.

### NK Cell or ILC1 Depletion In Vivo

In vivo, NK cells and ILC1s were depleted by i.p. injection with 200 μg/mouse anti-mouse NK1.1 antibody (clone PK136; BioXcell, USA); NK cells alone were depleted by i.p. injection with 40 μl/mouse anti-asialo-GM1 antibody (clone Poly21460; BioLegend, USA). To maintain the depletion, the same injections were given on days 7, 14, and 21.

### Gene Expression Analyses

For mouse ILC1 RNA-sequencing, mouse ILC1s were sorted from the liver of normal mice or mice with AML using BD FACSAria™ Fusion. For LSC RNA-sequencing, 2,000 mouse LSCs sorted from Mll^{PTD/WT}: Flt3^{ITD/ITD} mice with AML were co-cultured with 1,000 ILC or treated with 10 ng/ml IFN-γ for 3 days; then the LSCs were re-sorted using BD FACSAria™ Fusion. Total RNA was isolated from ILC1s or LSCs using a miRNeasy mini kit (QIAGEN). PolyA RNA-seq was performed in the Integrative Genomics Core of City of Hope National Medical Center. SMART-Seq® Ultra Low Input RNA Kit for Sequencing-v4 (Takara Bio) was used for getting double-strand cDNA from each sample with 2 ng of input total RNA. The resulting cDNA was sheared using a Covaris LE220 sonicator. The sheared DNA was used for to prepare a sequencing library, using a KAPA HyperPrep Kit. The final libraries were quantified using the Qubit Assay Kit (Thermo Fisher Scientific) and Bioanalyzer (Agilent). Sequencing was performed using the single-read mode of 51 cycles of read1 and 7 cycles of index read with V4 reagents on a Hiseq 2500 system (Illumina). Real-time analysis (RTA) 2.2.38 software was used to process the image analysis and base calling.

For quantitative (q)RT-PCR and regular PCR analyses, RNA was isolated from 1,000 cells using a miRNeasy mini kit (QIAGEN) and reverse-transcribed using the PrimeScript™ RT Reagent Kit with gDNA Eraser (TAKARA). qPCR reactions were run on a QuantStudio 7 Flex Real-Time PCR System (Applied Biosystems) using SYBR Green reagents (Thermofisher). Values were normalized to 18s rRNA expression levels. qPCR analysis was conducted to assess the expression of mouse Bak1 (Forward: 5′-CAGCTTGCTCTCATCGGAGAT-3′, Reverse: 5′-GGTGAAGAGTTCGTAGGCATTC-3′), human Bak1 (Forward: 5′-GTTTTCCGCAGCTACGTTTTT-3′, Reverse:5′-GCAGAGGTAAGGTGACCATCTC-3′), and 18S rRNA (Forward: 5′-GTAACCCGTTGAACCCCATT-3′; Reverse: 5′ -CCATCCAATCGGTAGTAGCG-3′). Regular PCR reactions to determine the expression of mouse Il7 (Forward: 5′-TTCCTCCACTGATCCTTGTTCT-3′, Reverse: 5′-AGCAGCTTCCTTTGTATCATCAC-3′) were performed on a ProFlex PCR System (Applied Biosystems) using 2xMyTaq Red Mix (Meridian Bioscience).

In Vitro Kinase Inhibitor Experiments

LSCs isolated from spleen of Mll^{PTD/WT}: Flt3^{ITD/ITD} mice with AML were treated with the JAK2 inhibitor AZD1480 (10 nM), the JAK1/2/3 inhibitor decernotinib (VX-509, 10 nM), or the AKT inhibitor afuresertib (10 nM) for 30 min. Then LSCs were co-cultured with ILC1s isolated from liver of WT or IFN-γ^{-/-} mice labeled with CTV at a ratio of 4:1, or treated with IFN-γ (10 ng/ml). Three days later, cells were harvested and analyzed using flow cytometry.

### ELISA

Cell supernatants were collected and analyzed for cytokine content by ELISA according to the manufacturer's protocols. LSCs isolated from the peripheral blood of patients with AML were co-cultured with the ILC1 s isolated from healthy donors or patients with AML in the presence of IL-12 (10 ng/ml) and IL-15 (100 ng/ml) for 3 days. Levels of IFN-γ in culture supernatants were measured using the human IFN-γ Quantikine ELISA Kit (Cat# DIF50C, R&D). Samples for each condition were assayed in three duplicates.

In vitro the Colony Forming Cell (CFC) Assay

1000 LSCs were obtained from Mll^{PTD/WT}/Flt3^{ITD/ITD} mouse spleens and co-cultured with or without 500 ILC1s for 3 days. Cells were then plated into mouse methylcellulose complete media (R&D, HSC007) supplied with human transferrin (200 μg/ml), recombinant human insulin (10 μg/ml), recombinant human SCF (50 ng/ml), murine recombinant IL-3 (10 ng/ml), IL-6 (10 ng/ml) and recombinant mouse Epo (5 IU/ml). Cultures were incubated at 37° C. in a humidified atmosphere of 5% CO2 for 10-14 days. Colony numbers were counted.

LSCs were obtained from Mll^{PTD/WT}: Flt3^{ITD/ITD} mouse spleen and co-cultured with or without WT, IFN-γ^{-/-} or TNF-α^{-/-} ILC1s for 3 days. Cells were then plated into mouse methylcellulose complete medium (R&D, HSC007) supplied with human transferrin (200 μg/ml), recombinant human insulin (10 μg/ml), recombinant human SCF (50 ng/ml), murine recombinant IL-3 (10 ng/ml), IL-6 (10 ng/ml), and recombinant mouse EPO (5 IU/ml). Cultures were incubated at 37° C. in a humidified atmosphere of 5% CO2 for 10-14 days. Colony numbers were counted using a microscope

mRNA Isolation and qPCR

Total mRNA was isolated using the RNeasy mini kit (QIAGEN) according to manufacturer's instructions. mRNA purity and quantity were determined with NanoDrop (Thermo Scientific) before RT-PCR and RNA-seq analysis. For RT-PCR, mRNA samples were reverse transcribed into cDNA using the High-Capacity cDNA Reverse Transcription Kit with RNase Inhibitor (Thermo Scientific).

### RNA-Sequencing

2,000 mouse LSCs were co-cultured with 1,000 ILC1s or treated with 1 ng/ml IFN-γ for 3 days, and then the LSCs were sorted using BD FACSAria Fusion. Total RNA of LSCs were isolated using miRNeasy mini kit (QIAGEN). SMART-Seq® Ultra Low Input RNA Kit for Sequencing-v4 was used for generating amplified double strand cDNA from each sample with 2 ng of input total RNA according to the manufacturer's protocol. The resulting double-stranded cDNA sheared with Covaris LE220 with the setting of DNA fragment size of 200 bp peak. The sheared DNA was used for sequencing library preparation by using KAPA HyperPrep Kits. The final libraries were quantified with qubit and bioanalyzer. The sequencing was performed with the single read mode of 51 cycles of read1 and 7 cycles of index read with V4 reagents on Hiseq2500. Real-time analysis (RTA) 2.2.38 software was used to process the image analysis and base calling.

### Inhibitors Experiment

1,000 LSCs isolated from spleen of Mll^{PTD/WT}/Flt3^{ITD/ITD} mice were treated with decernotinib (VX-509, 10 μM), AZD1480 (10 μM), or afuresertib (10 nM) for 30 min. Then 500 mouse ILC1s isolated from liver of WT or IFN-γ^{-/-} mice labeled by CTV or IFN-γ (10 ng/ml) were cocultured with LSCs. Three days later, cells were harvested and analyzed by flow cytometry.

### Statistical Analysis

Prism software v.8 (GraphPad, CA, USA) was used to perform statistical analysis. Two group comparisons were performed with a two-tailed Student's t-test; multiple group comparisons were performed with a one-way ANOVA test with a multiple comparisons option. For Kaplan-Meier survival curve analysis, the comparisons were performed using a log-rank (Mantel-Cox) test. For continuous endpoints, Student's t test was used to compare two independent conditions, and one-way ANOVA models were used to compare three or more independent conditions. For repeated measures over time, linear mixed models were used to account for the variance and covariance structure. Mouse survival was estimated by the Kaplan-Meier method and compared by log-rank tests. All tests were two-sided. P values were adjusted for multiple comparisons by Holm's procedure. For RNA-seq analysis, sequencing reads were trimmed from sequencing adapters using Trimmomatic⁷⁴ and polyA tails using FASTPT⁷⁵, and then mapped back to the mouse genome (mm10) using STAR (v. 020201)⁷⁶. The gene-level count table was created by HTSeq (v.0.6.0)⁷⁷ and normalized by the TMM⁷⁸ method. General linear models based on negative binomial distributions (R package “EdgeR”) were used to compare gene expression levels between two specific cell types. Genes with an FDR-adjusted p-value less than 0.05 and a fold change (FC) greater than 1.5 (upregulated) or less than 0.7 (downregulated) were considered as differentially expressed genes (DEG). Pathway and gene set enrichment analyses were performed using the GSEA^{79,80} program, which runs the GSEAPreranked algorithm on a ranked list of genes. Data are presented as mean±SD. Prism software v.8 (GraphPad, CA, USA) and SAS v.9.4 (SAS Institute. NC, USA) were used to perform statistical analyses. The p-values are represented as: *<0.05, **<0.01, ***<0.001, and ****<0.0001

### Example 1: ILC1s Induce Apoptosis of AML LSCs In Vitro

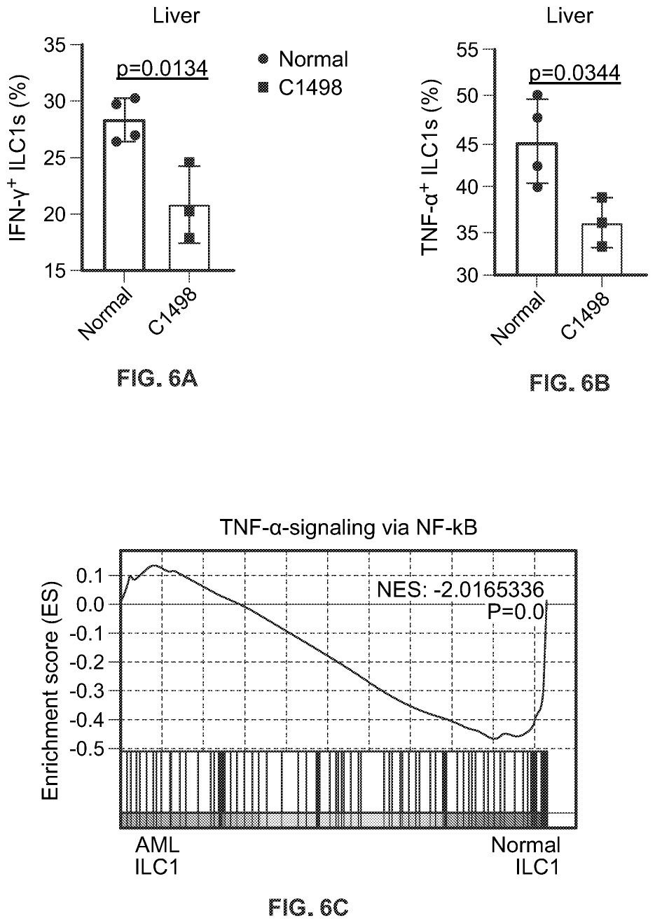

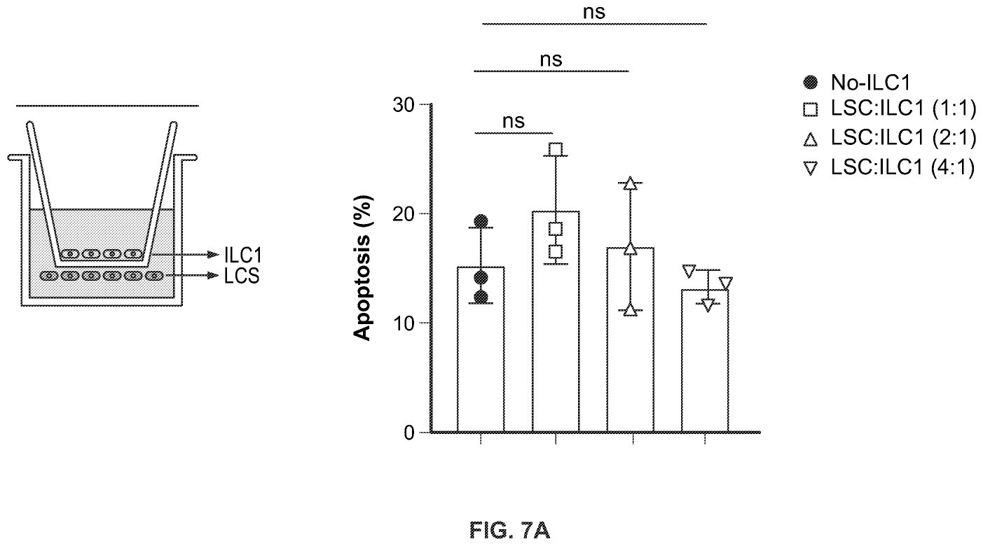

This example investigates the function of ILC1s in AML, or in cancer in general, which is largely unknown. Using mouse models of decreased production of IFN-γ and TNF-α in mice with AML compared to control mice show the function of ILC1s isolated from the liver was impaired (FIGS. 6A-6B). Consistent with this, RNA sequencing (RNA-seq) analysis of ILC1s indicated that nuclear factor-κB (NF-κB) signaling, a pathway that controls ILC1 function¹⁹, was also decreased in mice with AML (FIG. 6C). This suggested that AML seemed to exert an immunosuppressive effect on ILC1s.

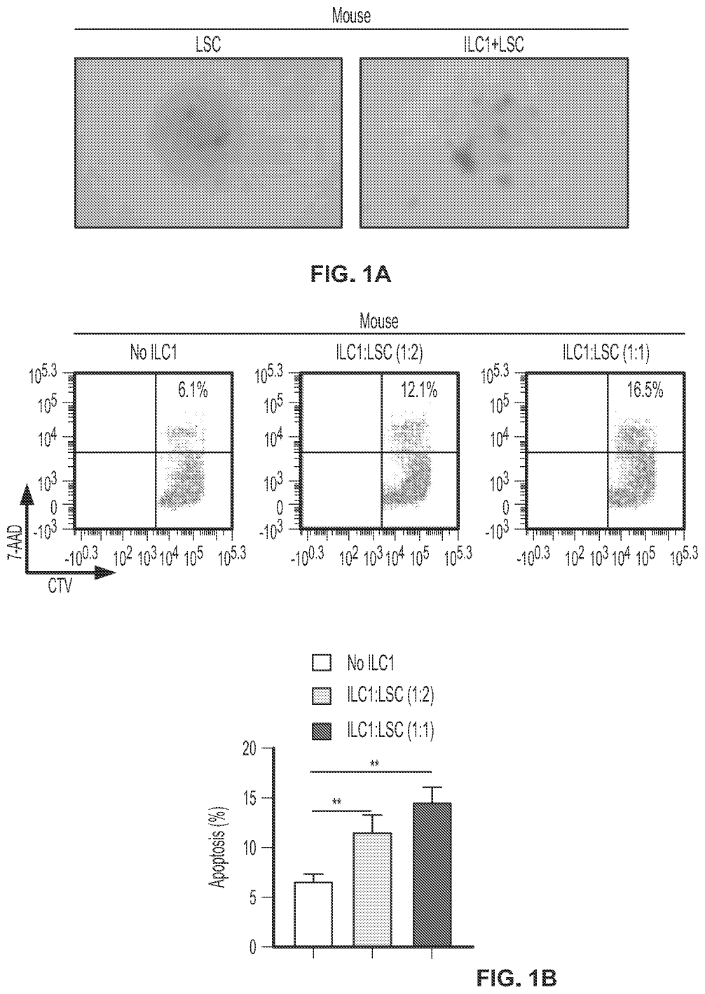

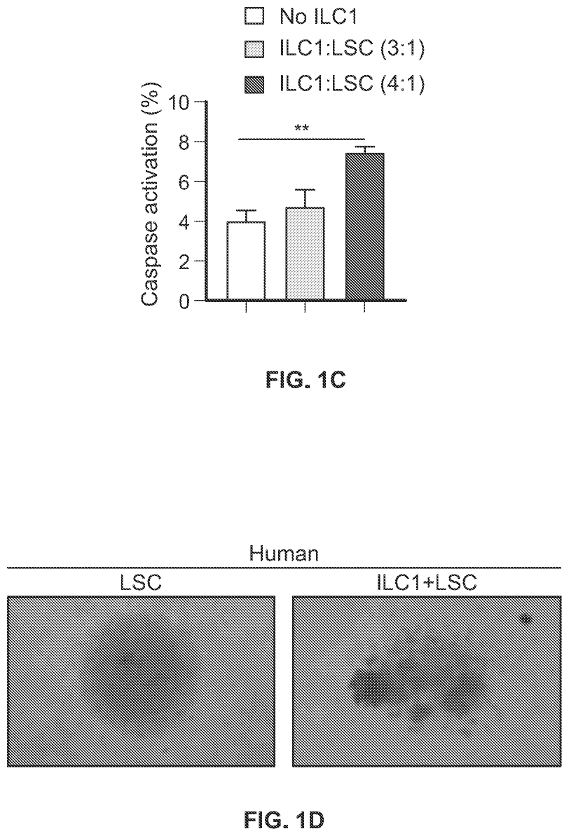

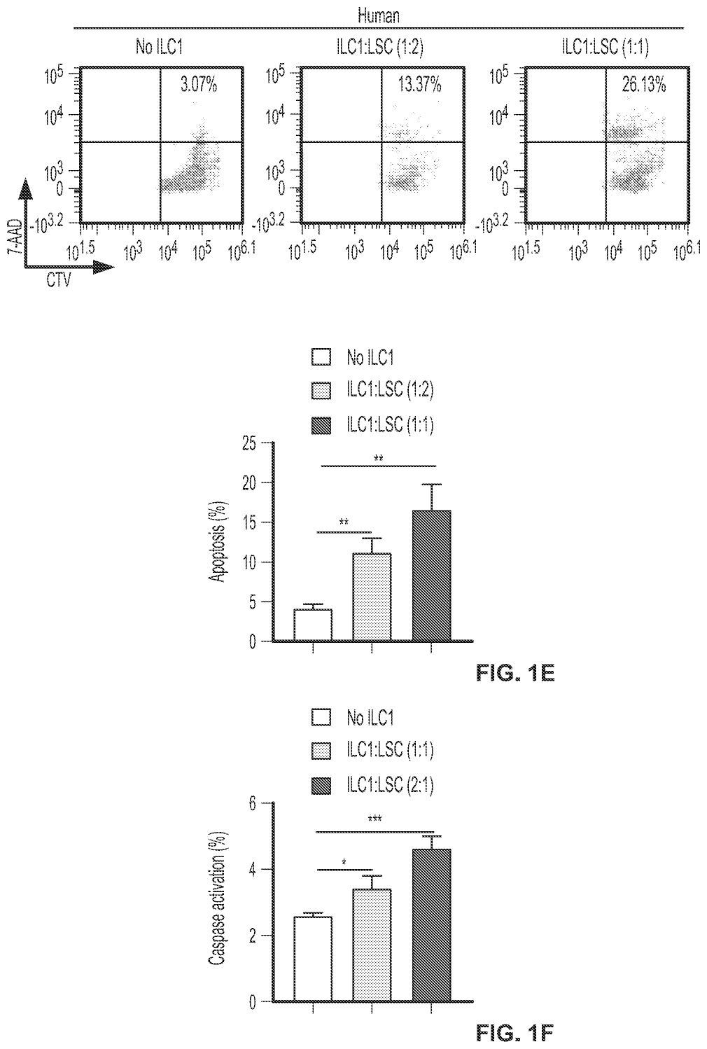

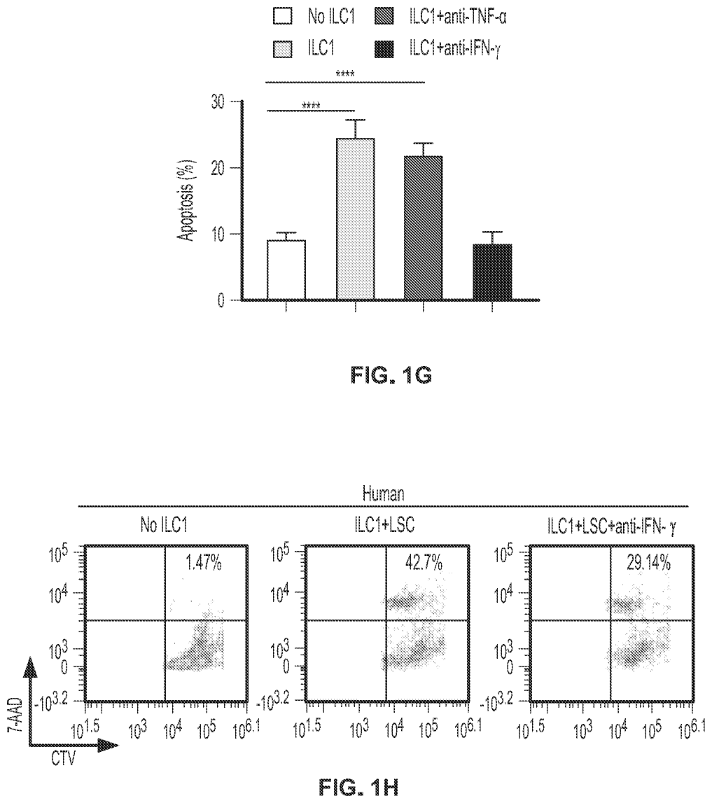

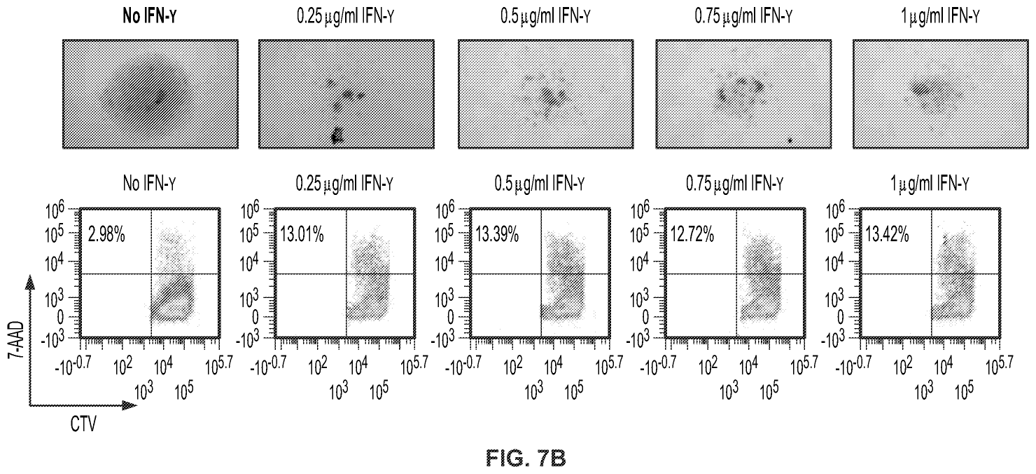

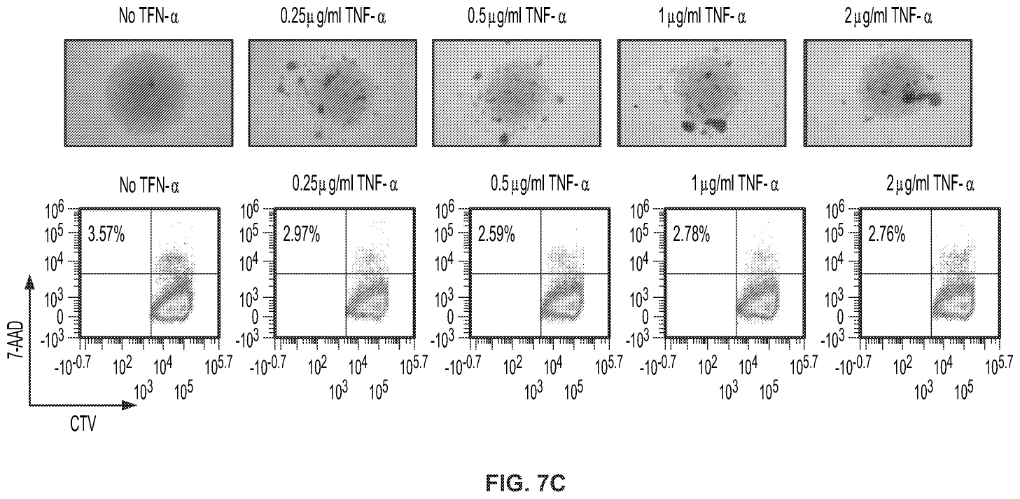

To investigate if ILC1s have an adverse effect on the genesis of AML, we conducted cell lysis analyses on AML cells after exposure to ILC1s. Sorted ILC1s from the livers of normal mice were co-cultured for 3 days with splenic LSCs (Lin -Sca-1⁺c-Kit⁺ cells)^{20,21} isolated from the Mll^{PTD/WT}/Flt3^{ITD/ITD} AML mouse model, previously generated and characterized by our group . Surprisingly, LSCs were lysed by ILC1s (FIGS. 1A-B), and caspase activation of LSCs, indicative of apoptotic cell death, significantly increased in 24 h after administering ILC1s (FIG. 1C). Similar results were achieved using ILC1s isolated from healthy human peripheral blood co-cultured with human LSCs isolated from the peripheral blood of AML patients (FIGS. 1D-1F). No statistically significant cell death of LSCs was observed when the ILC1s were separated by a transwell chamber (FIG. 7A). Taken together, these data suggest that ILC1s induce apoptotic cell death of LSCs and likely require cell-cell contact to do so.

Murine leukemia stem cells (LSCs or Lin⁻Sca-1⁺c-Kit⁺ cells) are found mainly in bone marrow (BM) and spleen in AML^{20,21}. Since ILC mainly reside in the liver, to investigate whether LSCs also reside in the liver of AML mice, we isolated LSKs from the liver of normal mice and Mll^{PID/WT}: Flt3^{ITD/ITD} mice with AML²² and then i.v. injected them into immunodeficient Rag2^{-/-}γc^{-/-} mice. We observed that all immunodeficient Rag2^{-/-}γc^{-/-} mice injected with LSKs isolated from the liver of normal mice lived, while all immunodeficient Rag2^{-/-}γc^{-/-} mice injected with LSKs isolated from the liver of mice with AML died, suggesting that LSCs are present in the liver of mice with AML (FIG. 21A). We validated our conclusion by using a transplantable murine LSCs model in which CD45.1 mice were injected with LSCs (CD45.2⁺ cells) isolated from the spleen of Mll^{PTD/WT}: Flt3^{ITD/ITD} mice with AML (FIG. 21B). Using flow cytometry, we showed that LSCs had been trafficked into the liver of CD45.1 mice (˜20% of total Lin⁻ cells) by 9 weeks after adoptive transfer (FIG. 21B). Our data are supported by a previous report 72 .

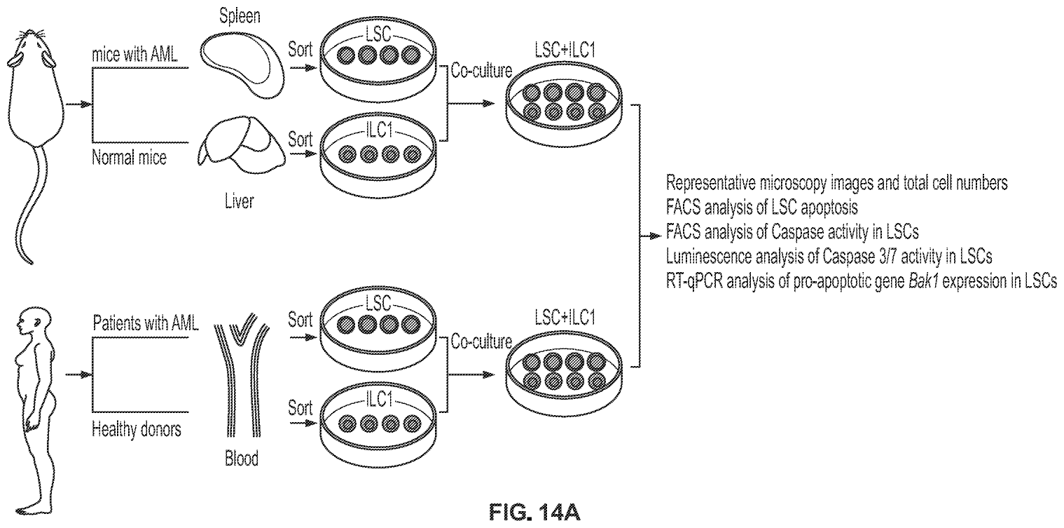

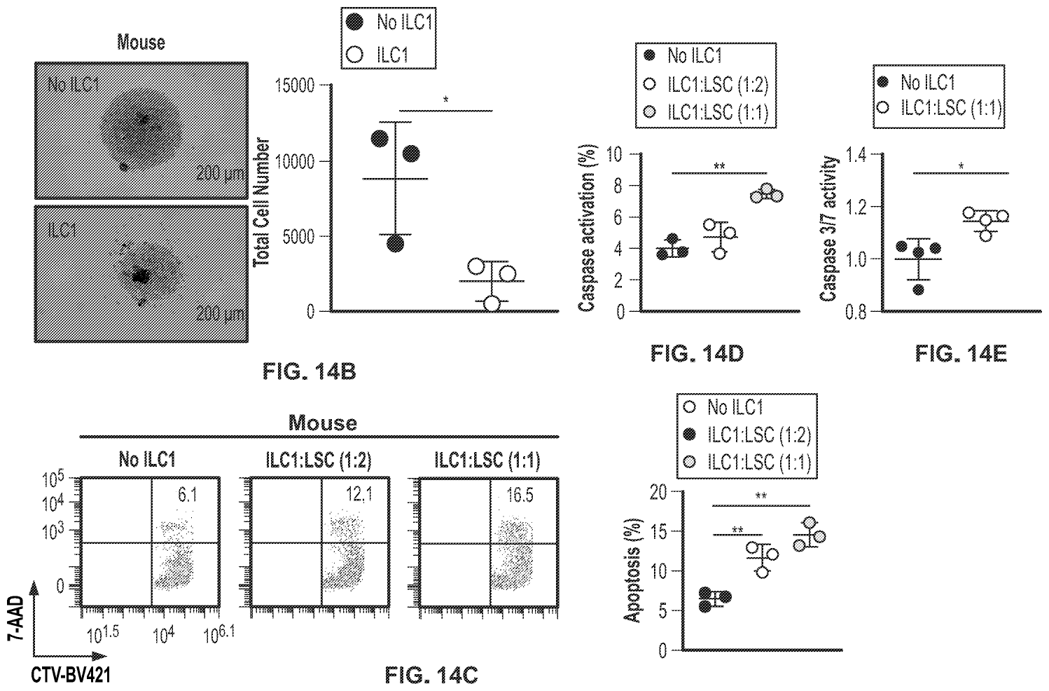

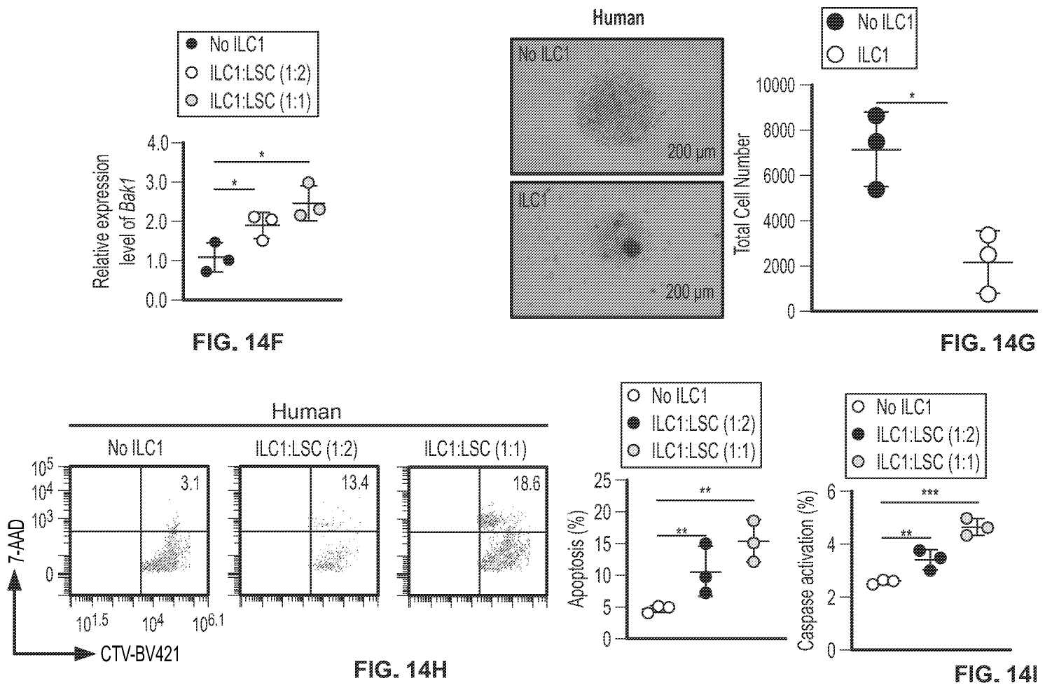

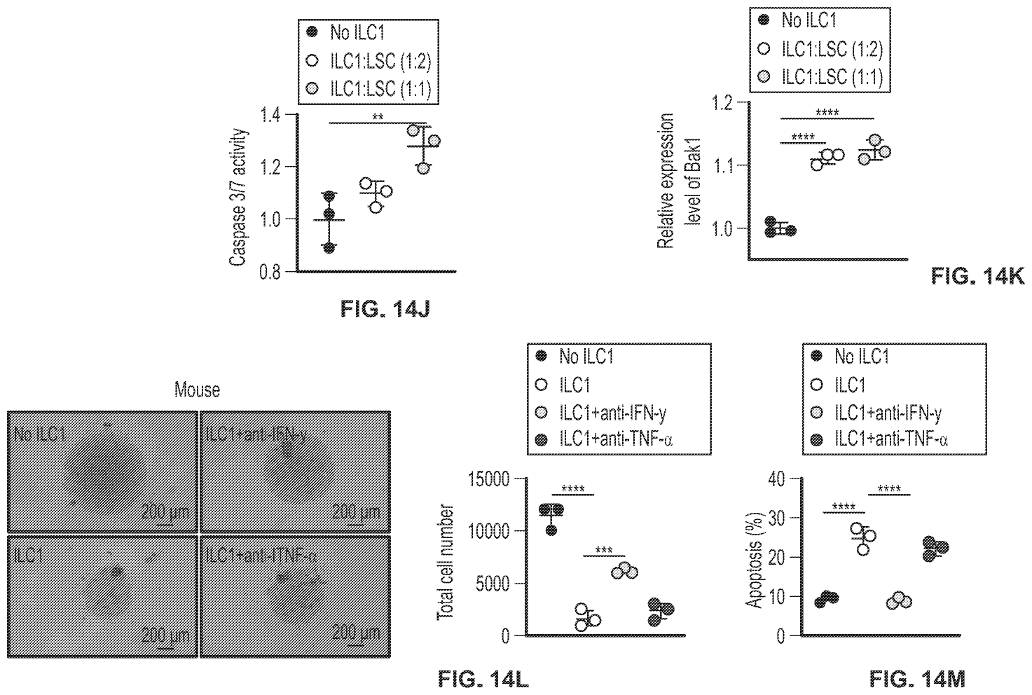

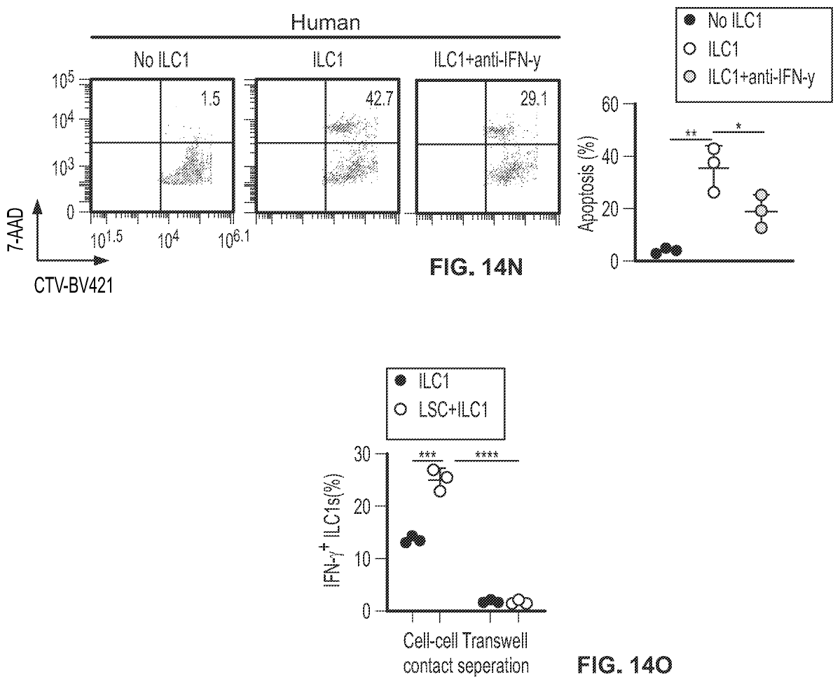

Using a mouse model of AML (C1498 cells i.v. injected into C57BL/6J mice)¹⁸, we noted that the function of ILC (Lin⁻NK1.1⁺NKp46⁺CD49b⁻CD49a⁺) (FIG. 21C) isolated from the liver and bone marrow, but not the spleen, was impaired, as indicated by decreased production of IFN-γ and TNF-α in mice with AML compared to normal mice (FIGS. 21E-21F). Consistent with this, RNA sequencing (RNA-seq) of ILC indicated that nuclear factor-κB (NF-κB) signaling, a pathway that controls ILC1 function¹⁹, was decreased in mice with AML (FIG. 21G). Given that AML seemed to exert an immunosuppressive effect on ILC1s, we asked if ILC have an adverse effect on the genesis of AML. To address this, we sorted ILC1s from the liver of normal mice and co-cultured them for 3 days with splenic LSCs isolated from the Mll^{PTD/WT}: Flt3^{ITD/ITD} mice with AML. The purity of LSCs and ILC1s was over 95% (FIGS. 22A-22B). LSCs were lysed by ILC1s at the ratio of 1:1 or 1:2 (FIGS. 14A-14C and FIG. 22C) as evidenced by a decrease in the absolute number of live LSCs and an increase in the fraction of apoptotic LSCs compared to co-culture without ILC1s. Additionally, total caspase and caspase3/7 activation of LSCs and the expression of the pro-apoptotic gene Bak1, indicative of apoptotic cell death, significantly increased in co-culture with ILC1s compared to co-culture without ILC1s (FIGS. 14D-14F and FIG. 22D). We achieved similar results using ILC1s (Lin⁻CD56⁻CD127⁺c-Kit⁻CRTH2⁻) isolated from healthy human peripheral blood (PB) that were co-cultured with human LSCs (CD45^{dim}Lin⁻CD34⁺CD38⁻) from PB of patients with AML (FIGS. 14H-14K and FIGS. 22E-22F), further suggesting that normal ILC1s induce apoptotic death of LSCs.

### Example 2: ILC1s Induce AML LSC Death Facilitated by Secretion of IFN-γ

ILC1s, which lack cytolytic activity, primarily function as immunoregulatory cells via their secretion of cytokines such as IFN-γ and TNF-α²³. To determine whether their production of either cytokine affects leukemogenesis, ILC1s and LSCs were co-culture in the presence of neutralizing antibodies against IFN-γ or TNF-α. In both mouse (FIG. 1G) and human (FIG. 1H) experiments, neutralization of IFN-γ but not TNF-α prevented or decreased ILC1-mediated induction of LSC death.

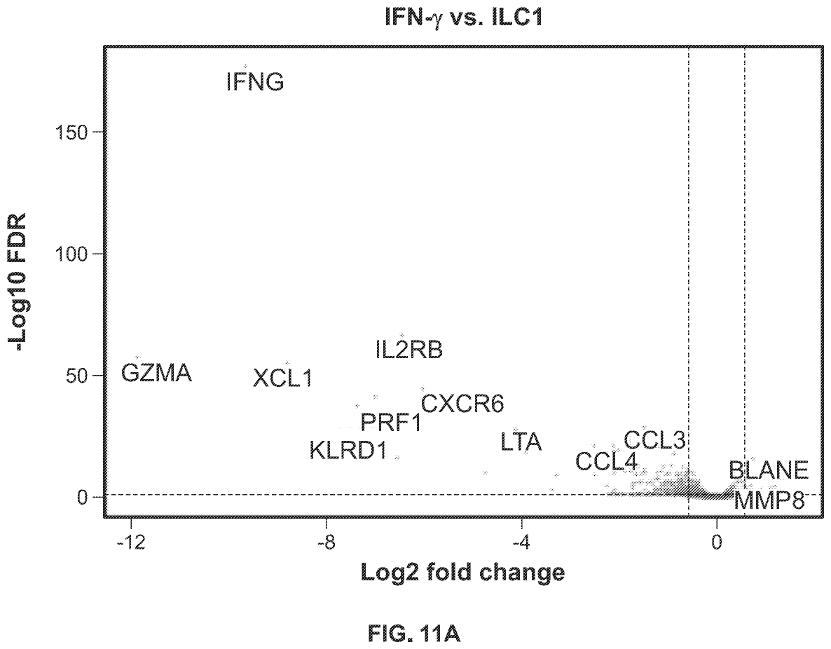

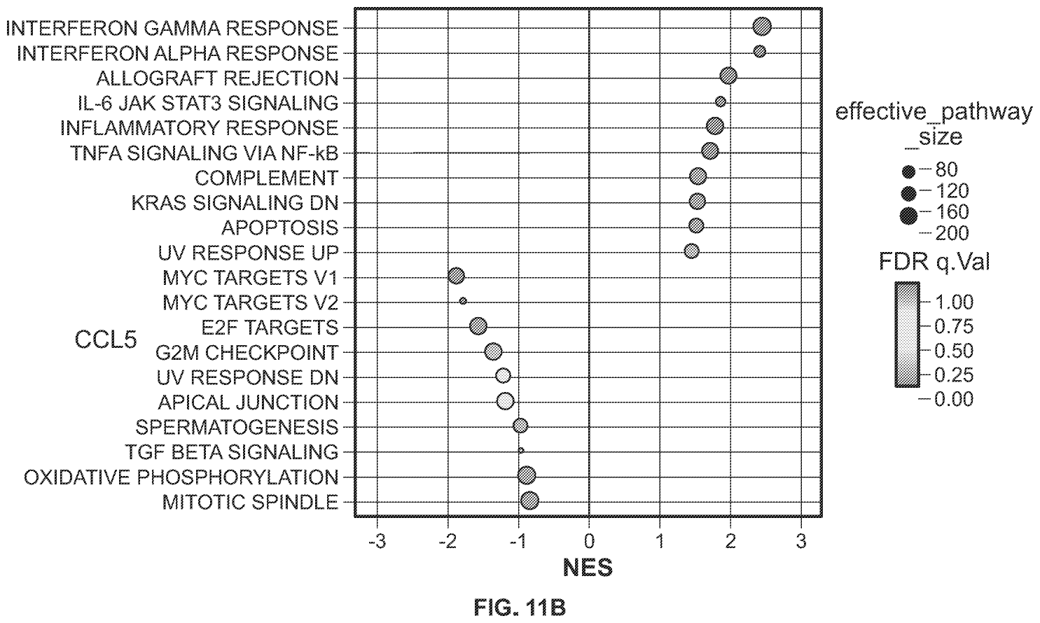

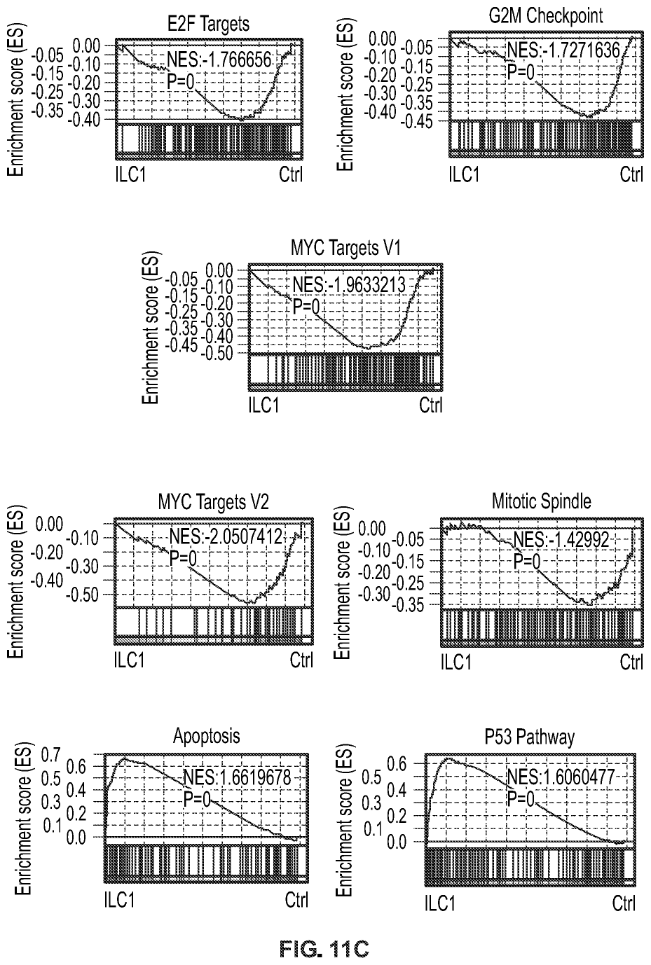

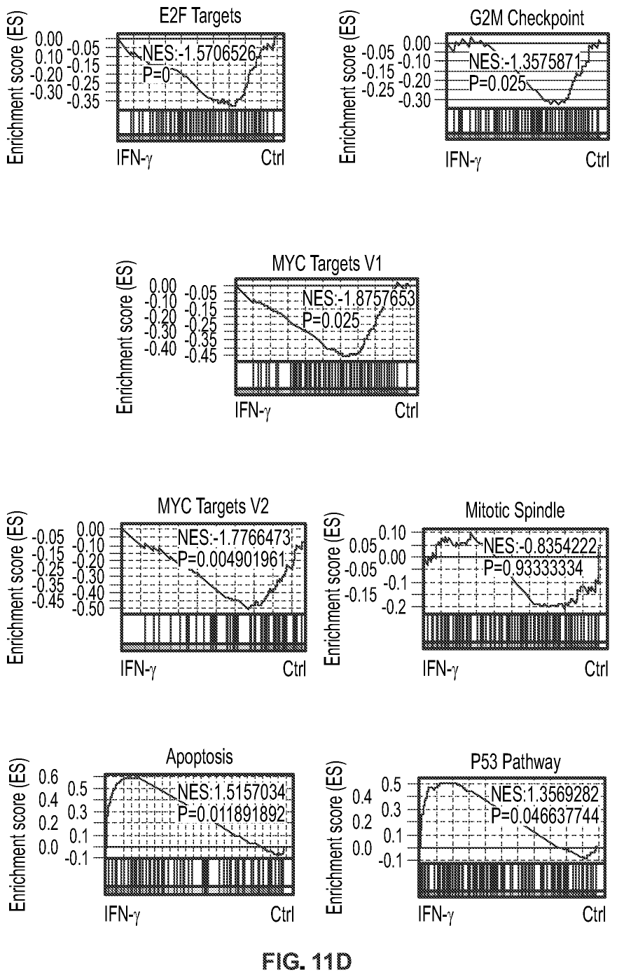

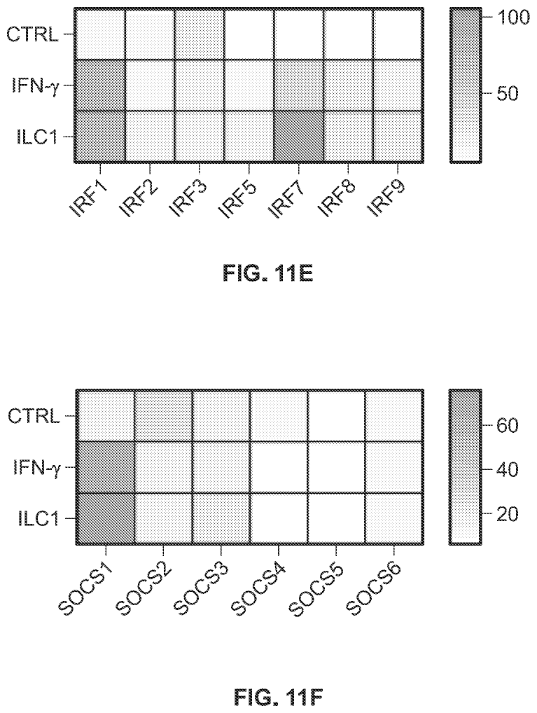

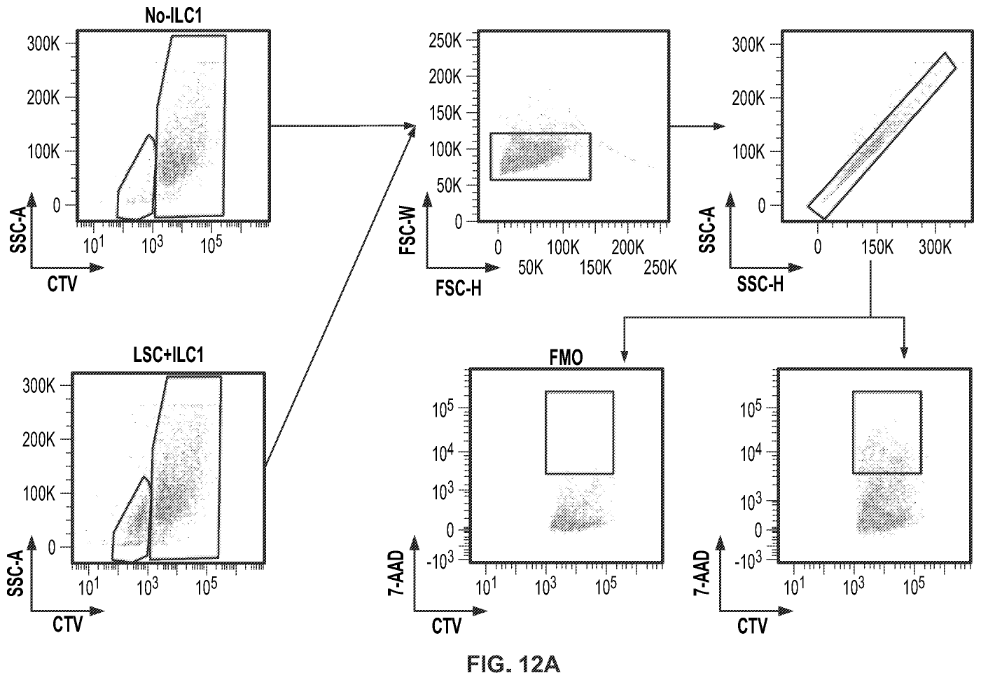

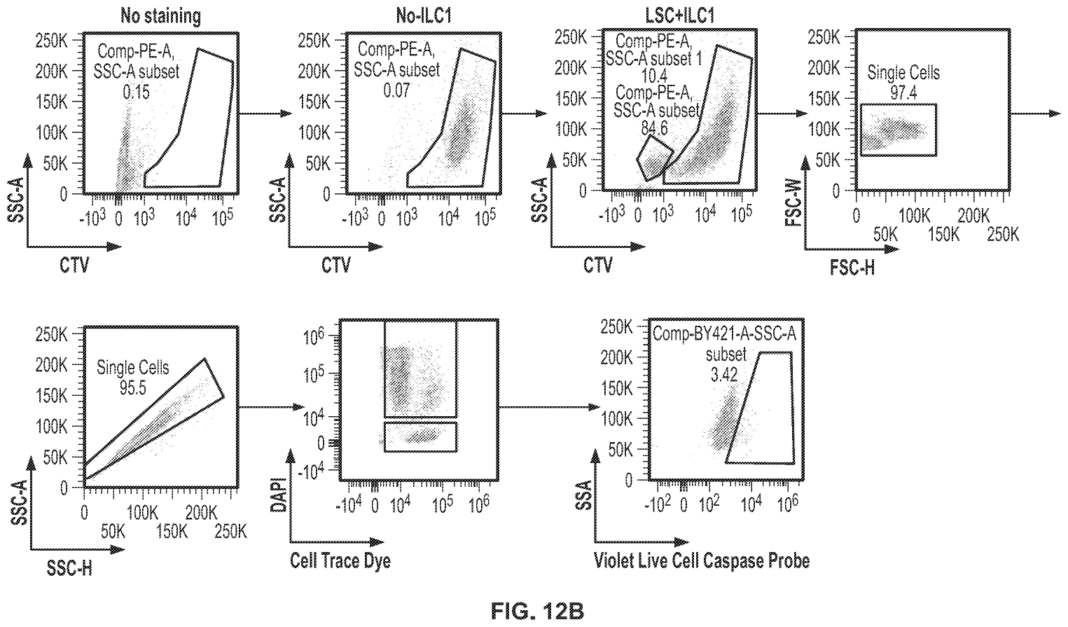

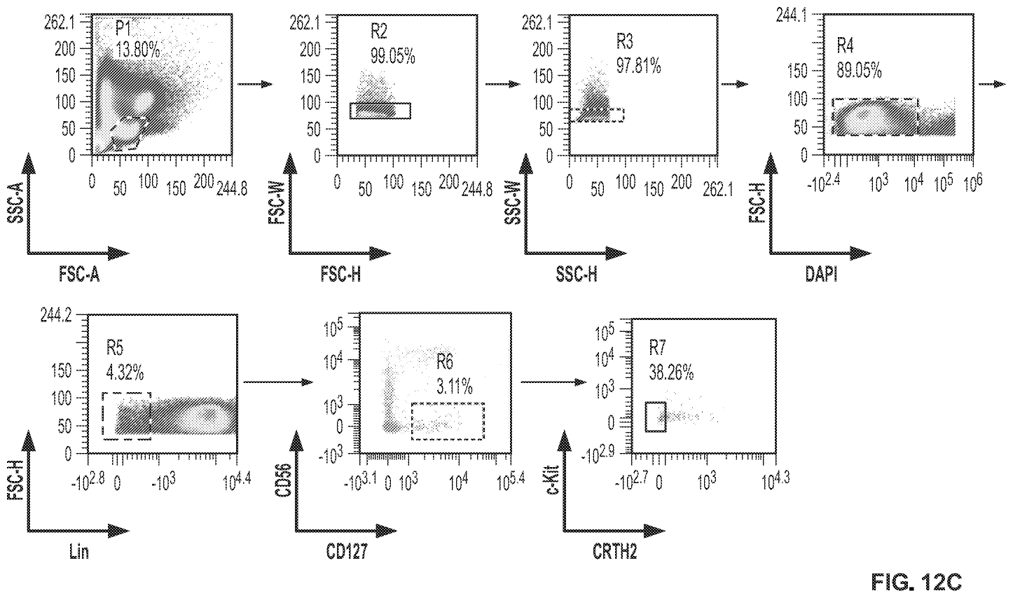

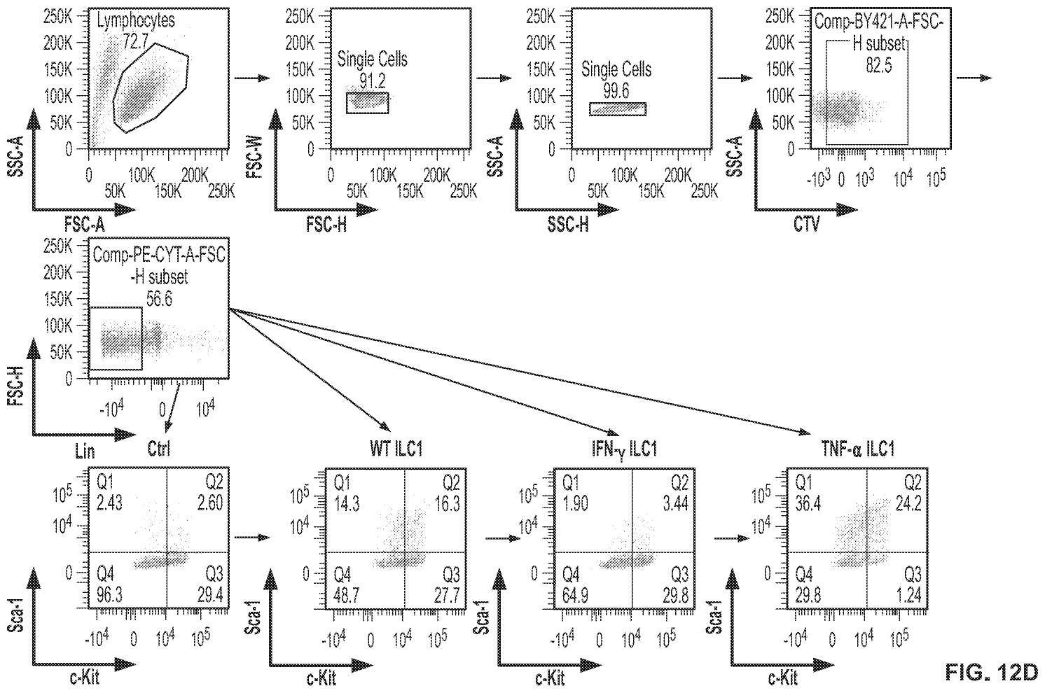

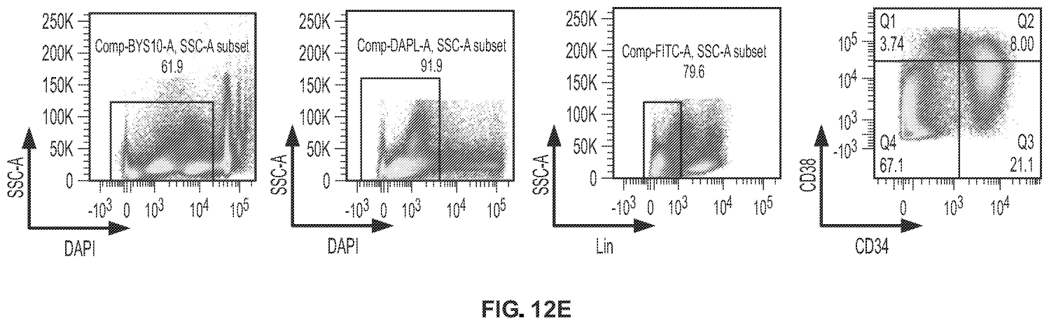

To determine whether cell-cell contact is required for induction of LSC apoptosis by ILC1s, ILC with LSCs were co-cultured using a transwell, in which ILC1s and LSCs were seeded in the upper and lower chambers, respectively. After three days of co-culture, ILC1s did not induce LSC apoptosis when separated by the transwell chamber (FIG. 7A). Production of IFN-γ in ILC was significantly increased after direct co-culture with LSCs (FIG. 11). The IFN-γ production in ILC1s was diminished using transwell separation (FIG. 11). To investigate whether LSC apoptosis requires a relatively high local concentration of IFN-γ, LSC experiments using recombinant murine IFN-γ instead of ILC1s. Recombinant murine IFN-γ induced LSC apoptosis (FIG. 7B), whereas recombinant murine TNF-α failed to do so (FIG. 7C).

The co-culture experiment was repeated using normal ILC1s and LSCs in the presence of neutralizing antibodies against IFN-γ or TNF-α. In that mouse experiment, neutralizing IFN-γ, but not TNF-α, prevented ILC1s from mediating the death of LSCs (FIGS. 14L-14M). This requirement for IFN-γ was validated using 30 human cells (FIG. 14N). To determine whether LSCs need cell-cell contact to induce apoptosis of LSCs, we co-cultured the two cell types separately in a transwell. After 3 days, we observed no LSC apoptosis (FIG. 23A). Strikingly, when the ILC1s and LSCs were co-cultured without the transwell so the cells could mingle, the ILC1s significantly increased their production of IFN-γ (FIG. 140). This observation suggests that ILC1s directly contacts LSCs to increase their IFN-γ output. Next, we validated the involvement of IFN-γ in inducing apoptosis of LSCs by using recombinant murine IFN-γ or TNF-α. We observed LSC apoptosis with IFN-γ (FIG. 23B) but not TNF-α (FIG. 23C). Collectively, our data demonstrate that cell-cell contact allows ILC1s to produce IFN-γ, which induces apoptosis of LSCs.

### Example 3: ILC1s and ILC1-Secreted IFN-γ Block Differentiation of LSCs into Leukemia Progenitor Cells

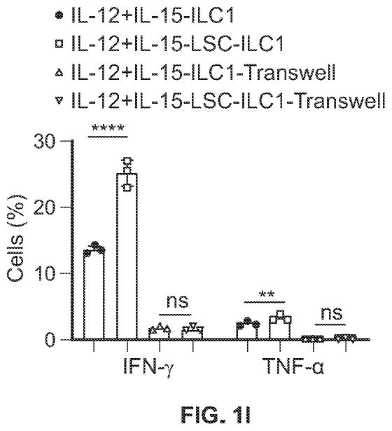

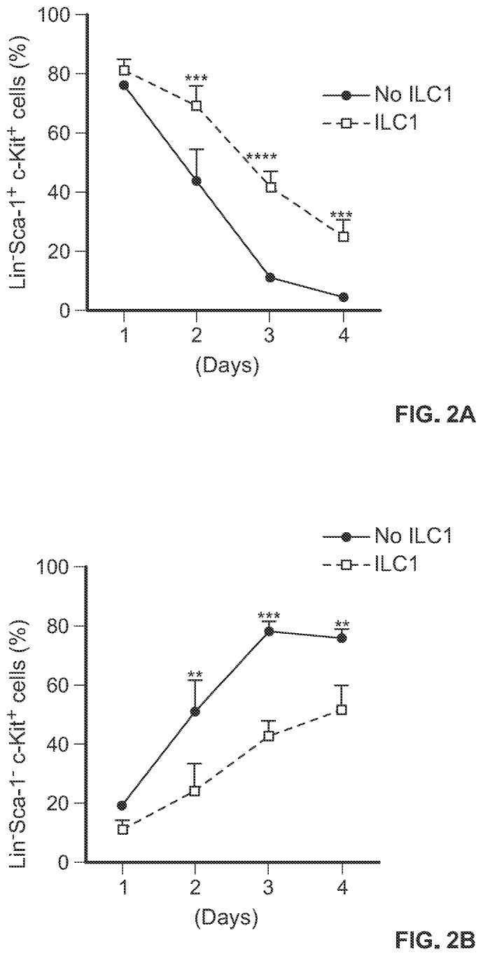

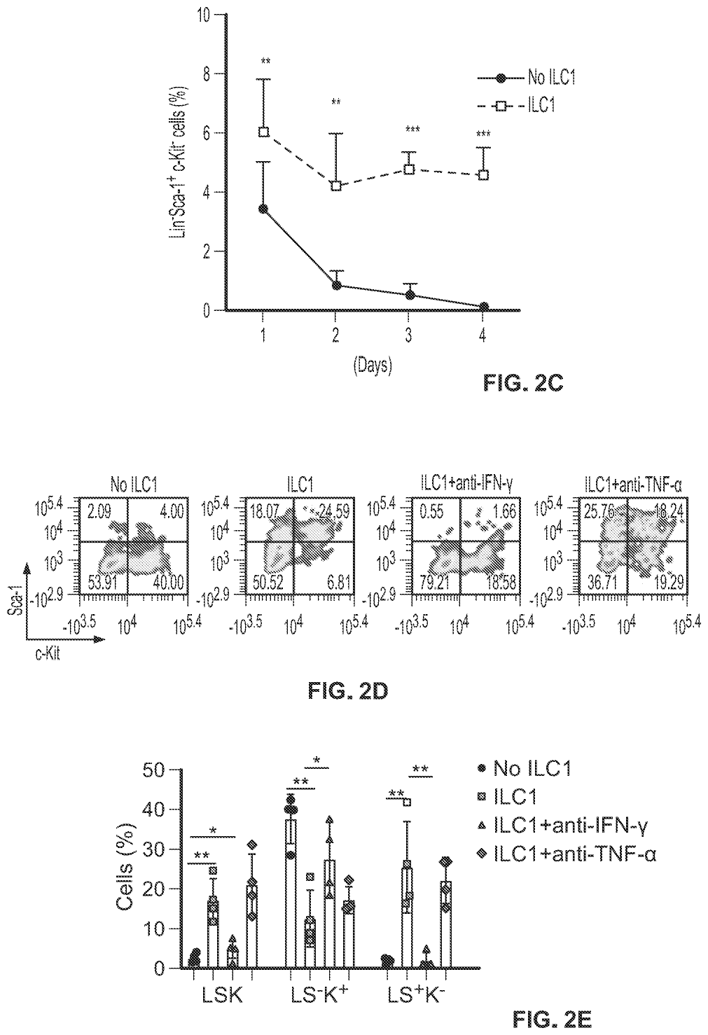

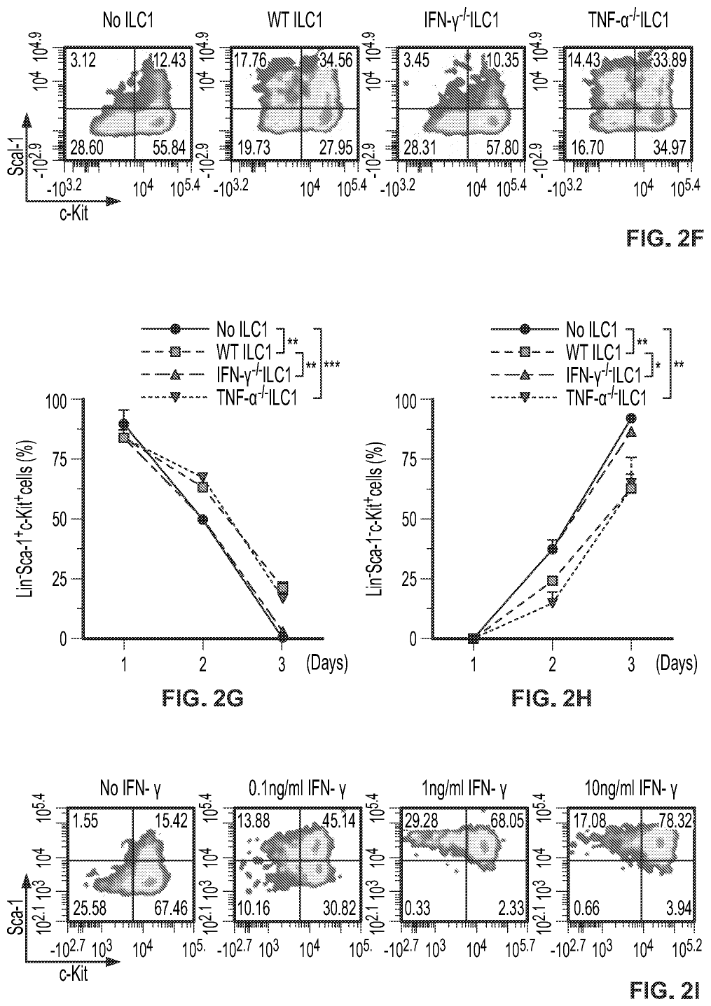

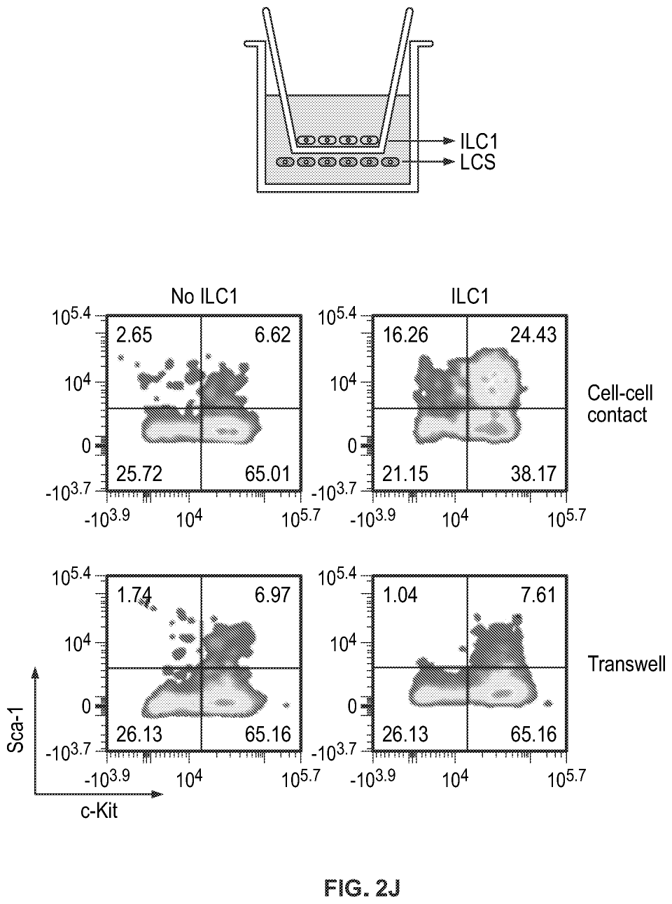

Initiation and differentiation of LSCs into leukemia progenitor cells drives the progression of AML^{24,25}. This example investigates the effects of ILC1s on the process of AML cell differentiation. LSCs isolated from the spleen of Mll^{PTD/WT}/Flt3^{ITD/ITD} AML mice were co-cultured with ILC1s isolated from the livers of mice for 1, 2, 3, and 4 days. On days 2, 3, and 4, the percentage of Lin⁻Sca-1⁻c-Kit⁺ leukemia progenitor cells (LS⁻K⁺ cells) was significantly lower in the group co-cultured with ILC compared to the group cultured without ILC (FIG. 2B). Further, the percentage of Lin⁻Sca-1⁺c-Kit⁻ non-leukemic cells (LS⁺K⁻ cells) was significantly increased after co-culture with ILC1s (FIG. 2C). Of note, previous studies demonstrated that non-leukemic LS⁺K⁻ cells contain early lymphoid-committed precursors in normal mice' and are highly apoptotic in mice with chronic myelogenous leukemia (CML)²⁷.

ILC1s inhibit differentiation of LSCs into LS⁻K⁺ leukemia progenitor cells while promoting differentiation of LSCs into non-leukemic LS⁺K⁻ cells. To determine how ILC1s inhibit differentiation of LSCs into LS⁻K⁺ leukemia progenitor cells and promote differentiation into non-leukemic LS⁺K⁻ cells, neutralizing antibodies against

IFN-γ and TNF-a were added to the ILC1—LSC co-culture. The IFN-γ neutralizing antibody blocked both ILC1-mediated suppression of LSC differentiation into LS⁻K⁺ leukemia progenitor cells and induction of LSC differentiation into non-leukemic LS⁺K⁻ cells (FIGS. 2D-2E). In contrast, TNF-a neutralizing antibody did not significantly change the ILC1 effect on differentiation. The findings were validated by co-culturing LSCs with ILC isolated from IFN-γ^{-/-} or TNF-α^{-/-} mice compared with ILC isolated from wild-type (WT) mice. ILC isolated from IFN-γ^{-/-} mice did not block differentiation of LSCs into LS⁻K⁺ leukemia progenitor cells and nor promote differentiation into non-leukemic LS⁺K⁻⁶⁰ cells (FIGS. 2F-2H). In contrast, ILC1s isolated from TNF-α^{-/-} mice promoted differentiation into non-leukemic LS⁺K⁻ cells and blocked differentiation of LSCs into LS⁻K⁺ leukemia progenitor cells, similar to ILC1s from WT mice (FIGS. 2F-2H).

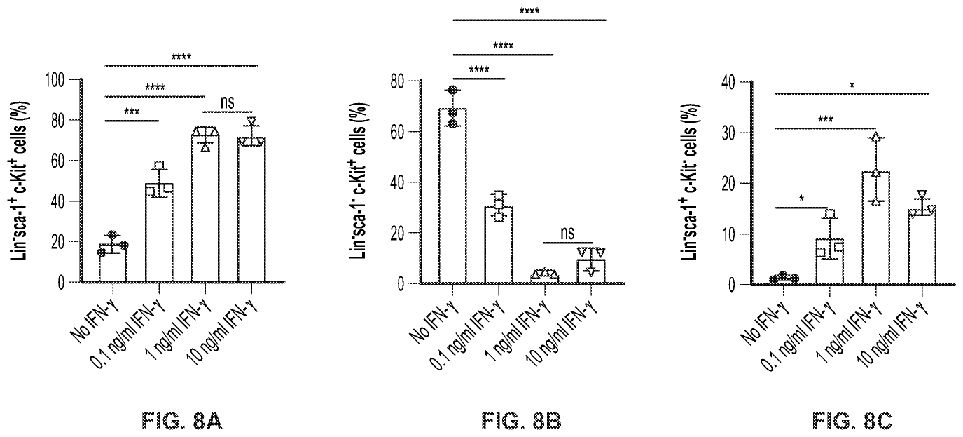

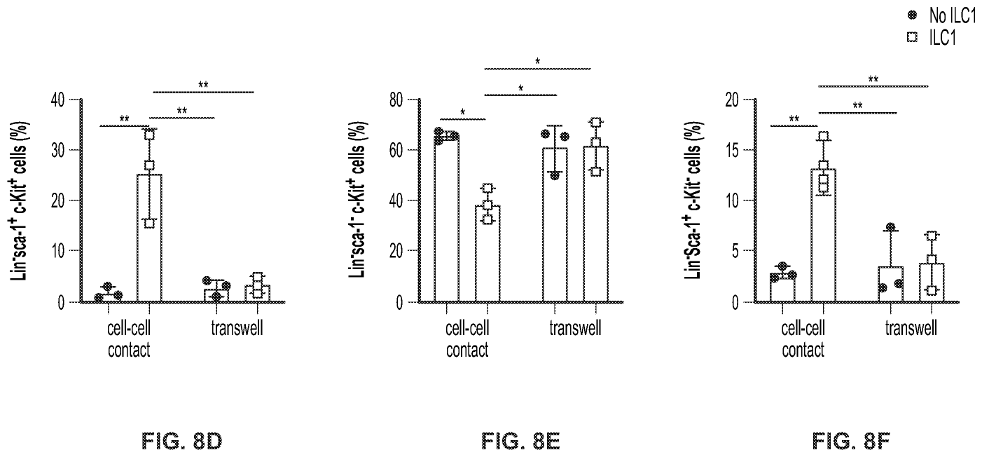

To investigate the role of IFN-γ produced by ILC1s to mediate these effects on LSCs, LSCs were incubated with recombinant murine IFN-γ. Similar to the ILC1-LSC co-culture, recombinant murine IFN-γ blocked differentiation of LSCs into LS⁻K⁺ leukemia progenitor cells and facilitated differentiation of LSCs into non-leukemic LS⁺K⁻ cells (FIG. 21, FIGS. 8A-8C). To determine if ILC1s regulate LSC differentiation through a cell-cell contact-dependent manner, LSCs were separated from ILC1s using a transwell chamber. The percentages of LSCs, LS⁻K⁺ cells, and LS⁺K⁻ cells varied between LSCs cultured directly with and without ILC1s (FIG. 2J, right, top; FIGS. 8D-8F); in contrast, the percentages did not differ between LSCs separated from ILC1s by a transwell and LSCs cultured without ILC1s (FIG. 2J right, bottom; FIGS. 8D-8F). Cell-cell contact is required to block the differentiation of LSCs into LS⁻K⁺ leukemia progenitor cells and promote their differentiation into non-leukemic LS⁺K⁻ cells. IFN-γ secreted by ILC1s also facilitates regulating LSC differentiation.

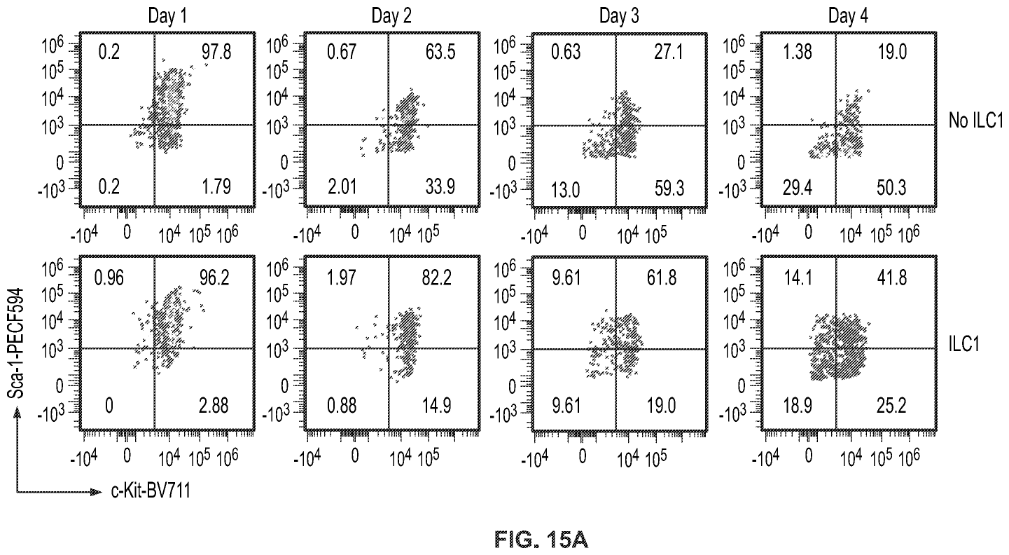

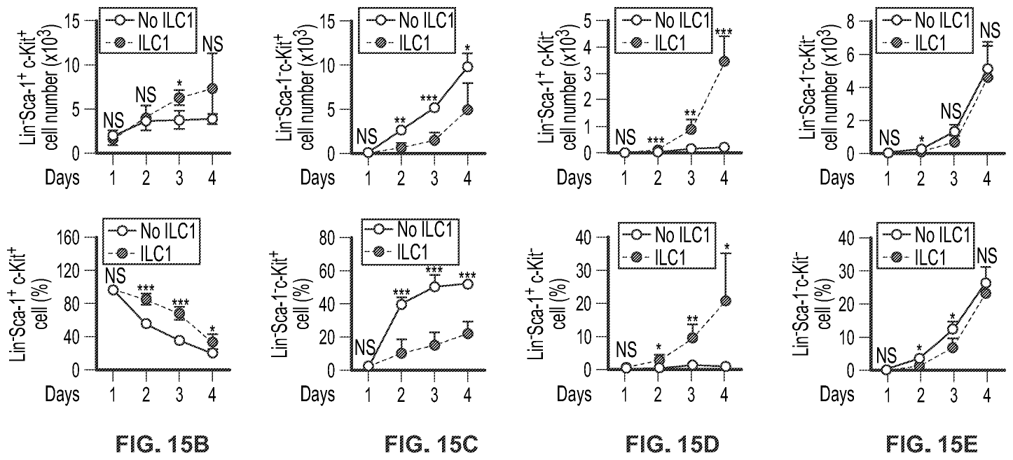

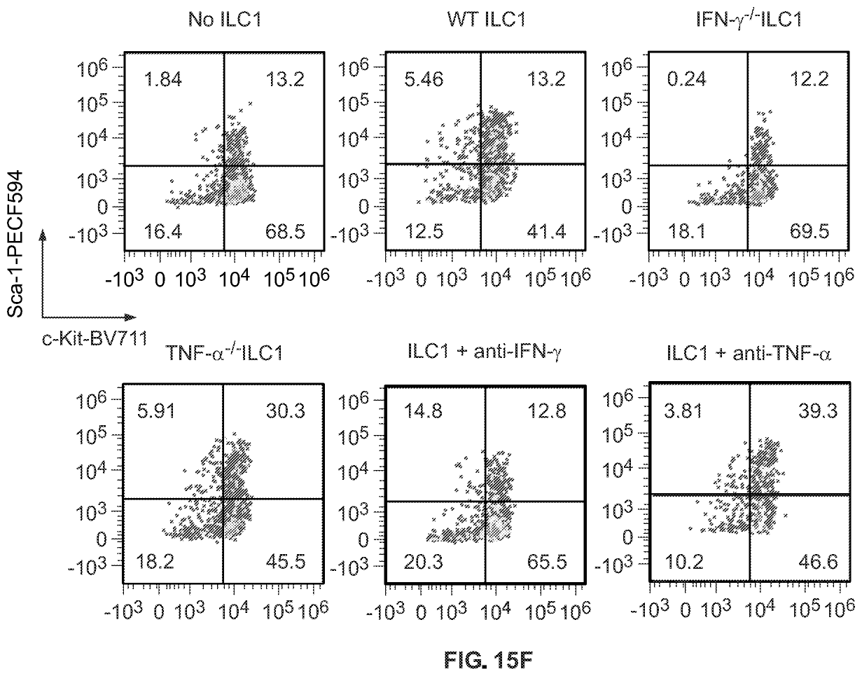

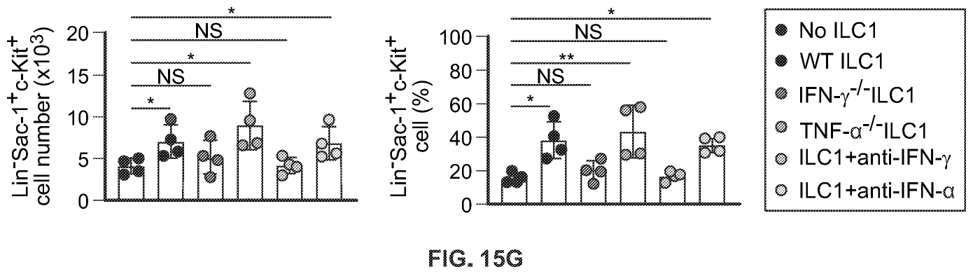

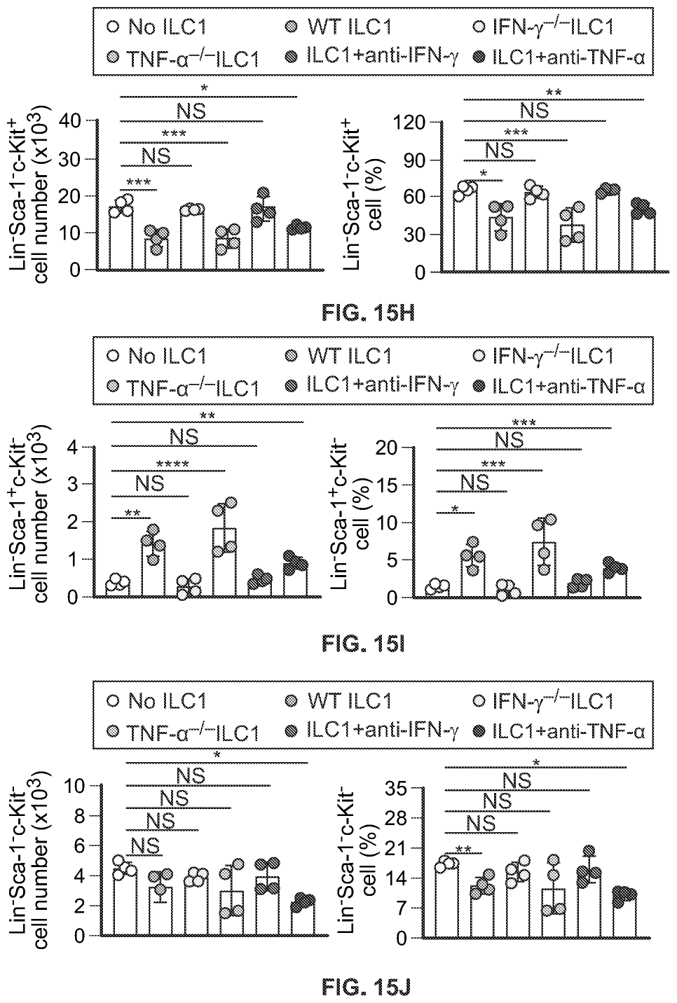

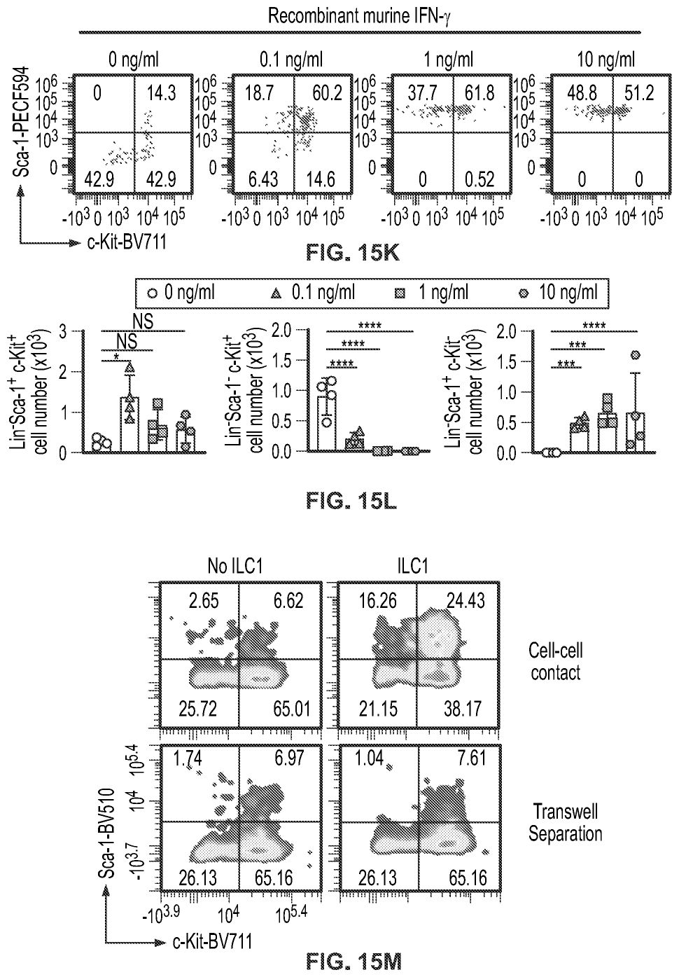

Experiments assessed the effects of ILC1s on LSC differentiation. For this purpose, we co-cultured LSCs isolated from the spleen of Mll^{PTD/WT}: Flt3^{ITD/ITD} mice with AML with ILC1s isolated from the liver of normal mice for 4 days. The ratio of ILC1s: LSCs was 1:4, which was lower than in the apoptosis assay. On days 3 and 4, the percentages and absolute numbers of LSCs were higher, whereas the percentages and the absolute numbers of Lin⁻Sca-1⁻c-Kit⁺ leukemia progenitor cells (LS⁻K⁺ cells) were significantly lower in the group co-cultured with ILC1s compared to the group co-cultured without ILC1s (FIGS. 15A-15C). Furthermore, the percentages and absolute cell numbers of Lin⁻Sca-1⁺c-Kit⁻ non-leukemic cells (LS⁺K⁻ cells)²⁷ was significantly higher after co-culture with ILC1s (FIG. 15D). No obvious difference was found in the Lin⁻Sca-1⁻c-Kit⁻ cell population (FIG. 15E). Of note, previous studies demonstrated that non-leukemic LS⁺K⁻ cells of normal mice contain early lymphoid-committed precursors²⁶ that are highly apoptotic in mice with chronic myelogenous leukemia (CML)²⁷. Our data indicate that ILC1s inhibit the differentiation of LSCs into LS⁻K⁺ leukemia progenitor cells while promoting their differentiation into non-leukemic LS⁺K⁻ cells. To determine the mechanism, we included neutralizing antibodies against IFN-γ and TNF-α in an ILC1—LSC co-culture. IFN-γ but not TNF-α neutralizing antibody inhibited both ILC1-mediated suppression of LSC differentiation into LS⁻K⁺ leukemia progenitor cells and induction of LSC differentiation into non-leukemic LS⁺K⁻ cells (FIGS. 15F-15J). We validated these data by comparing LSCs co-cultured with ILC1s isolated from IFN-γ^{-/-} or TNF-α^{-/-} mice with ILC1s isolated from wild-type (WT) mice. ILC1s isolated from IFN-γ^{-/-} mice could no longer inhibit the differentiation of LSCs into LS⁻K⁺ leukemia progenitor cells or promote their differentiation into non-leukemic LS⁺K⁻ cells. In contrast, ILC isolated from TNF-α^{-/-} mice acted similarly to ILC from WT mice (FIGS. 15F-15J). To further confirm that IFN-γ produced by ILC1s mediates these effects on LSCs, we incubated LSCs with recombinant murine IFN-γ. Same as for ILC1-LSC co-culture, recombinant murine IFN-γ inhibited the differentiation of LSCs into LS⁻K⁺ leukemia progenitor cells and facilitated their differentiation into non-leukemic LS⁺K⁻ cells (FIGS. 15K-15L and FIG. 24A).

To determine if ILC1s regulate LSC differentiation through cell-cell contact (as thought to be critical for LSC apoptosis), we separated LSCs and ILC1s in a transwell chamber. As expected, the percentages of LSCs, LS⁻K⁺ leukemia progenitor cells, and LS⁺K⁻ non-leukemic cells varied between LSCs cultured with and without 20 ILC1s (FIG. 15M, top; FIG. 24B). In contrast, the percentages were similar whether LSCs were separated from ILC1s by a transwell or cultured without ILC1s (FIG. 15M, bottom; FIG. 24B). These data suggest that cell—cell interaction is required to inhibit the differentiation of LSCs into LS⁻K⁺ leukemia progenitor cells and to promote their differentiation into non-leukemic LS⁺K⁻ cells. Taken together, our data indicate that IFN-γ secreted by ILC plays a key role in regulating LSC differentiation.

### Example 4: ILC1s and ILC1-Secreted IFN-γ Block Differentiation of LSCs into Terminal Myeloid Blasts

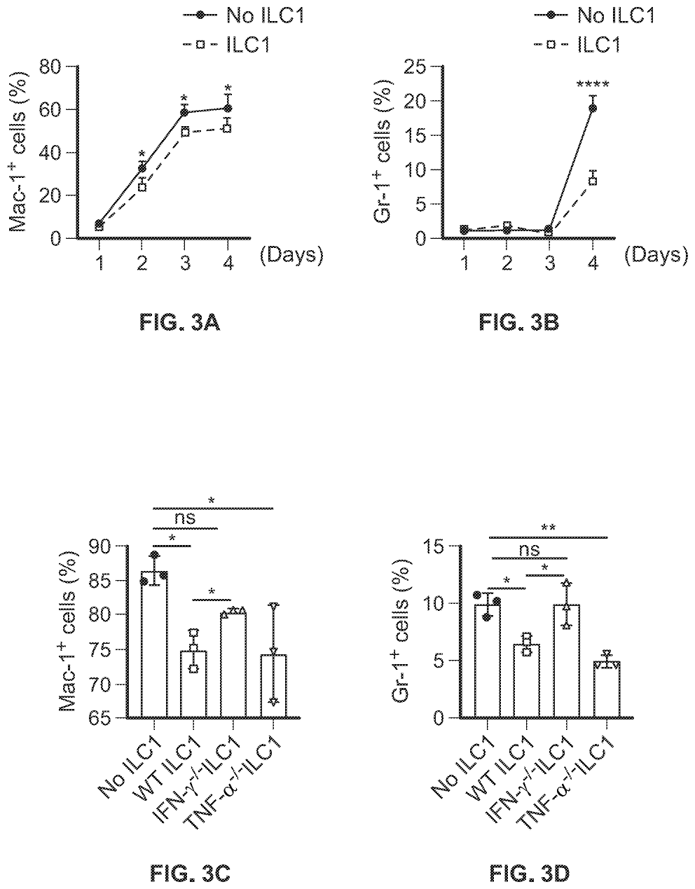

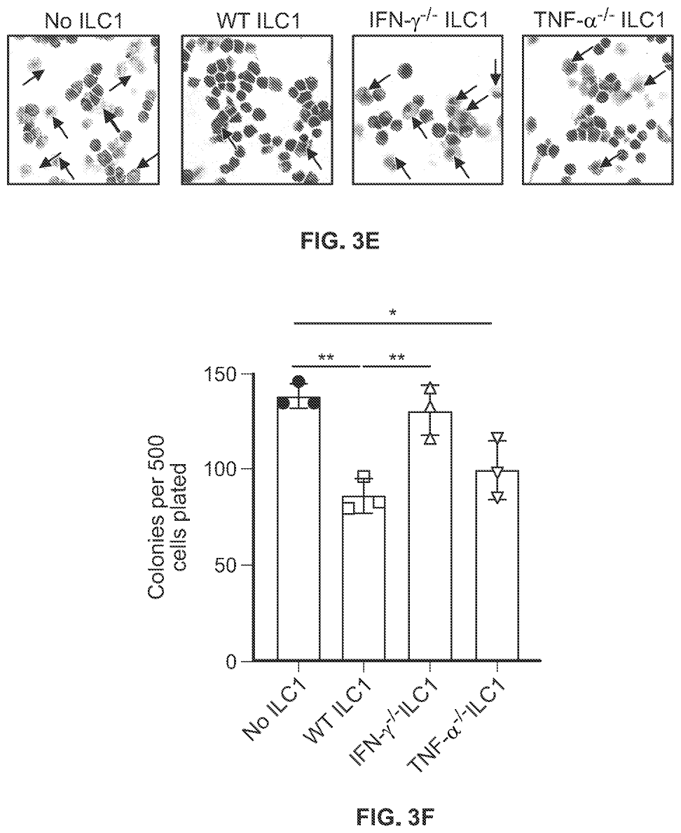

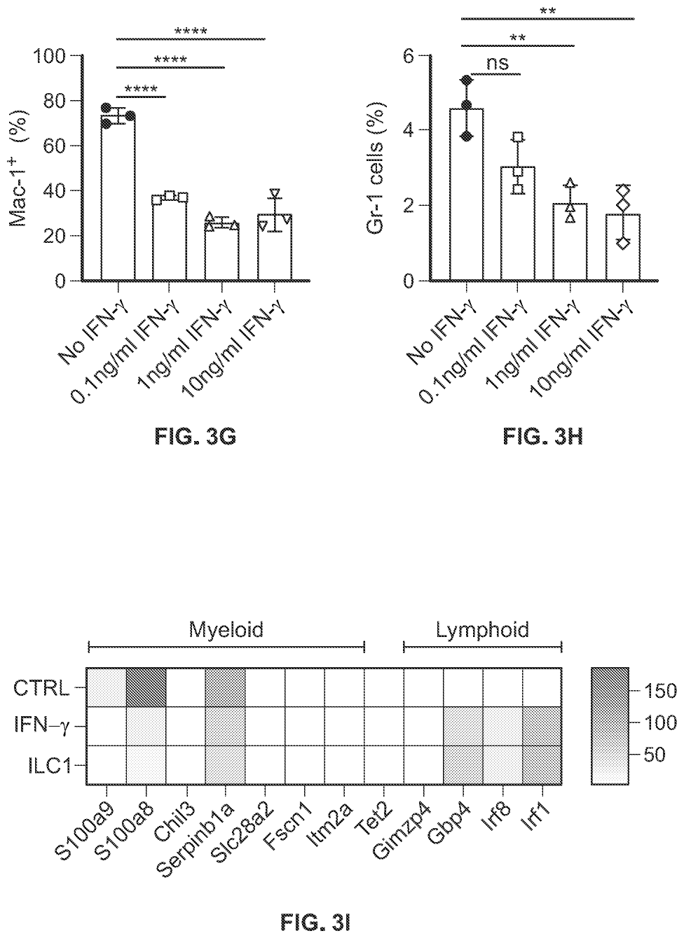

LSCs are capable of differentiating into normal myeloid cells and malignant blasts²⁸⁻³⁰. To determine whether ILC1s affect LSCs differentiation into terminal myeloid blast cells, LSCs were co-cultured with ILC1s for 1, 2, 3, and 4 days. ILC1s significantly inhibited LSC differentiation into terminal myeloid blasts, as shown by reduced populations of cells expressing macrophage-1 antigen (Mac-1) and the myeloid differentiation antigen Gr-1 compared to LSCs alone (FIGS. 3A-3B). When LSCs were co-cultured with IFN-γ^{-/-} or TNF-α^{-/-} ILC1s, the populations of cells expressing Mac-1 and the myeloid differentiation antigen Gr-1 significantly increased in co-culture with IFN-γ^{-/-} ILC but did not change in co-culture with TNF-α^{-/-} ILC1s as compared to WT ILC1s (FIGS. 3C-3D). To validate this data, an independent histological analysis was performed. The number of cells with differentiated morphology decreased when LSCs were co-cultured with WT ILC1s, compared to LSCs cultured with no ILC or with IFN-γ^{-/-} ILC1s, whereas the number of differentiated cells was unchanged between LSCs cultured with WT ILC1s and with TNF-α^{-/-} ILC1s (FIG. 3E).

A colony-forming unit assay starting with an equal number of LSCs was also performed. LSCs cultured with IFN-γ^{-/-} ILC1s formed similar numbers of colonies as LSCs cultured without ILC1s, whereas LSCs cultured with WT or TNF-α^{-/-} ILC1s formed significantly fewer colonies (FIG. 3F). To investigate the role IFN-γ produced by ILC1s in LSC differentiation into terminal myeloid blasts, LSCs were treated with recombinant murine IFN-γ. The IFN-γ suppressed differentiation of LSCs into Mac-1⁺ and Gr-1⁺ cells (FIGS. 3G-3H). Additionally, RNA-seq analysis of LSCs co-cultured with ILC or recombinant IFN-γ was performed. Compared to untreated LSCs, LSCs co-cultured with ILC1s or IFN-γ exhibited reduced expression of S100a9, S100ab, Chil3, Serpinb1a, and Slc28a2 genes, which are associated with myeloid differentiation^{31,32} (FIG. 3I). LSCs treated with ILC1s or IFN-γ also exhibited increased expression of Gpb4 and interferon regulatory factor (Irf)8 and 1 genes, which are associated with lymphoid differentiation³³ (FIG. 3I).

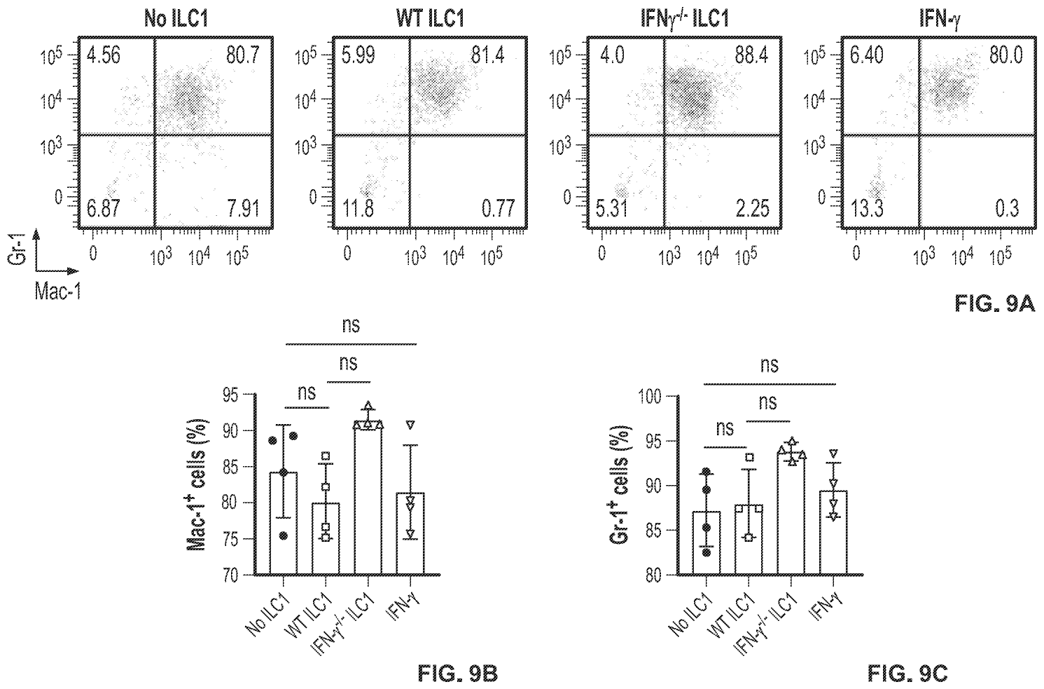

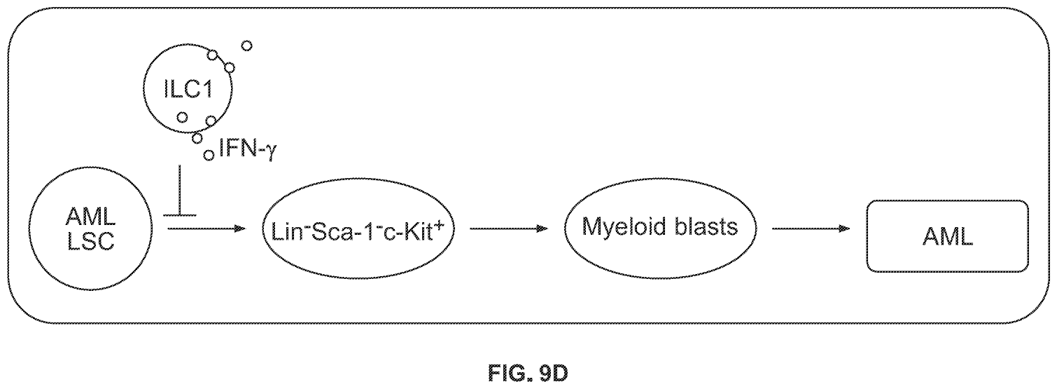

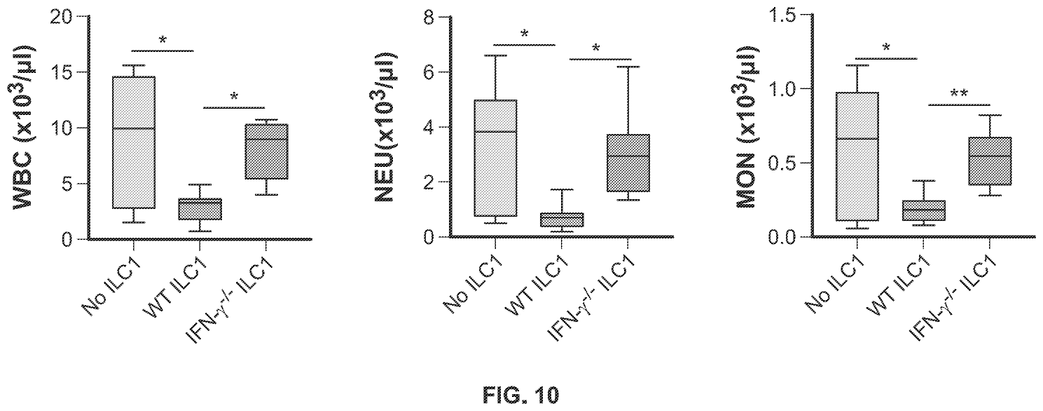

The process of LSC differentiation into AML blasts includes transitions from LSCs to LS⁻K⁺ leukemia progenitor cells, and from LS⁻K⁺ leukemia progenitor cells to AML blasts. To investigate which part of the process was affected by ILC1 and IFN-γ, LS⁻K⁺ leukemia progenitor cells were sorted from Mll^{PTD/WT}/Flt3^{ITD/ITD} AML mice, then the LS⁻K⁺ leukemia progenitor cells were treated with WT ILC1, ILC1s, or recombinant IFN-γ for 5 days. There was no statistical difference in the percentage of Mac-1⁺ and Gr-1⁺ cells among any of the groups (FIGS. 9A-9C). Thus, ILC1s block LSC differentiation into AML blasts, likely via a process involving by IFN-γ, during the transition from LSCs into LS⁻K⁺ leukemia progenitor cells, upstream of progenitor cell differentiation into AML blasts (FIG. 9D).

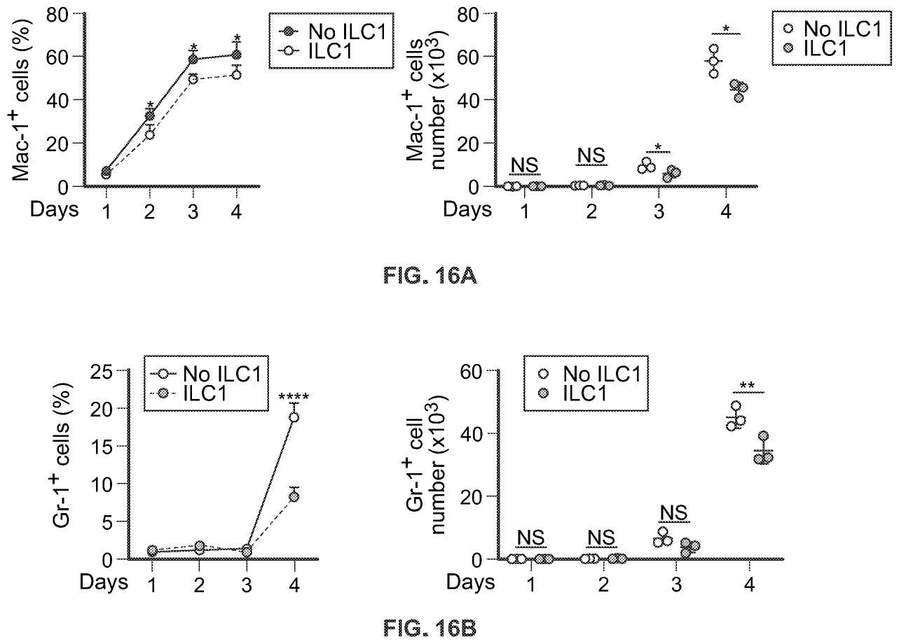

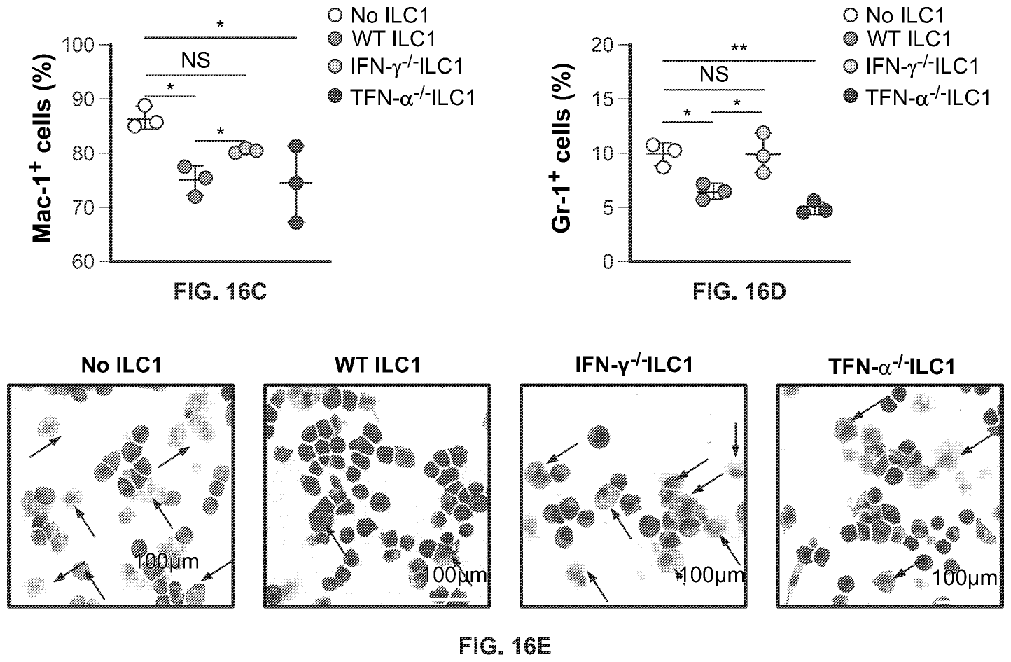

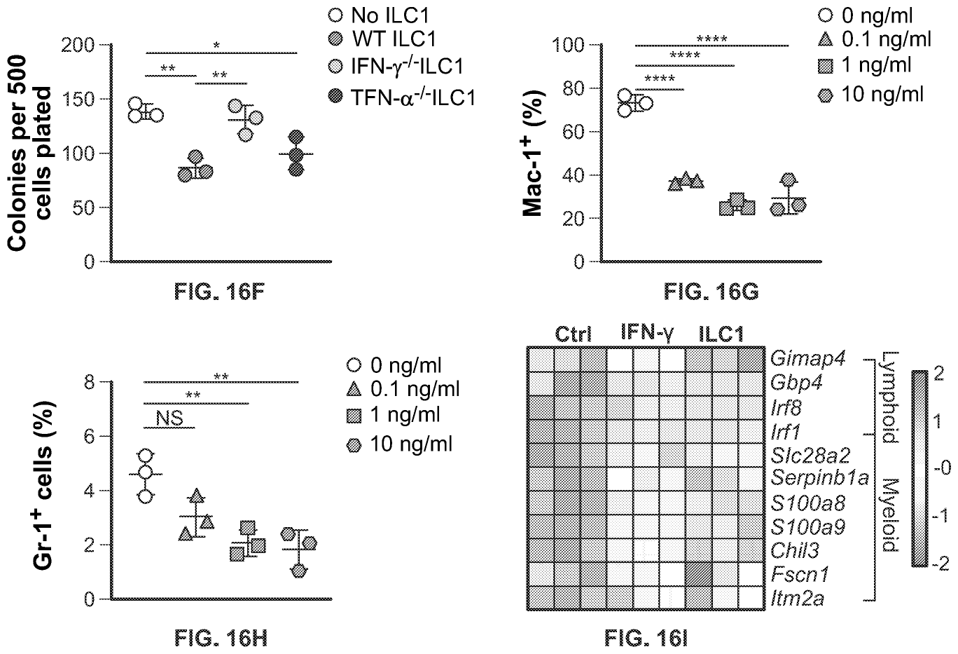

LSCs are hierarchical cells that can give rise to the terminal myeloid blasts that sustain AML²⁸⁻³⁰. To determine whether ILC1s affect the differentiation of LSCs into terminal myeloid blasts, we co-cultured LSCs with normal ILC1s for 1, 2, 3, or 4 days. On days 3 and 4, the ILC1s had significantly inhibited LSC differentiation into terminal myeloid blasts (compared to no ILCs), as indicated by reduced populations of cells expressing macrophage-1 antigen (Mac-1) and the myeloid differentiation antigen Gr-1 (FIGS. 16A-16B). When we co-cultured LSCs with IFN-γ^{-/-} or TNF-α^{-/-} ILC1s and compared them with co-cultures of LSCs with WT ILC1s, we observed significantly increased populations of cells expressing Mac-1 and Gr-1 in the co-culture with IFN-γ^{-/-} ILC1s—but not in the co-culture with TNF-α^{-/-} ILC1s (FIGS. 16C-16D). We obtained similar results when we co-cultured normal ILC1s with LSCs in the presence of neutralizing antibodies against IFN-γ or TNF-α (FIGS. 25A-25B). To validate this data, we performed an independent histological analysis, which demonstrated that the number of cells with differentiated morphology decreased when LSCs were co-cultured with WT ILC1s or TNF-α^{-/-} ILC1s, compared to LSCs cultured with no ILC or with IFN-γ^{-/-} ILC1s, whereas the population of differentiated cells was unchanged between LSCs co-cultured with WT ILC1s and with TNF-α^{-/-} ILC1s (FIGS. 16E). We also performed a colony-forming unit assay, starting with an equal number of LSCs. Compared to LSCs co-cultured without ILC1s, those co-cultured with IFN-γ^{-/-} ILC1s formed a similar number of colonies, whereas LSCs co-cultured with WT or TNF-α^{-/-} ILC1s formed significantly fewer colonies (FIG. 16F). To confirm that IFN-γ produced by ILC1s mediates LSC differentiation into terminal myeloid blasts, we treated LSCs with recombinant murine IFN-γ. This treatment suppressed the differentiation of LSCs into cells expressing Mac-1 and Gr-1 (FIGS. 16G-16H). Additionally, we cultured LSCs with ILC1s, with recombinant IFN-γ, or with no treatment (control). After the ILC1-LSC co-culture, we separated the LSCs from the ILC1s using FACS and then performed RNA-seq analysis. Compared to untreated LSCs, LSCs co-cultured with ILC1s or treated with IFN-γ reduced their expression of S100a8, S100a9, Chil3, Serpinb1a, and Slc28a2 genes, which associate with myeloid differentiation^{31-32, 71}. In contrast, we observed increased expression of Gimap4, Gpb4, and the interferon regulatory factor genes Irf8 and Irf1 (FIG. 16I), which associate with lymphoid differentiation^{33,70}.

LSCs transition into LS⁻K⁺ leukemia progenitor cells before becoming AML blasts. To investigate which step in this sequence is affected by ILC1s and IFN-γ, we sorted LS⁻K⁺ leukemia progenitor cells from Mll^{PTD/WT}: Flt3^{ITD/ITD} mice with AML, and then treated them with WT or IFN-γ^{-/-} ILC1s or recombinant IFN-γ for 5 days. The percentages of cells expressing Mac-1 and Gr-1 remained constant among the groups (FIG. 25C).

The data indicate that ILC1s suppress LSC differentiation into AML blasts via a process mediated by IFN-γ. This suppression occurs during the first transition from LSCs into LS⁻K⁺ leukemia progenitor cells—rather than during the subsequent step that converts progenitor cells into AML blasts (FIG. 25D).

### Example 5: ILC1s and ILC1-Secreted IFN-γ Control Leukemia Development and Prolong the Survival of Leukemic Mice

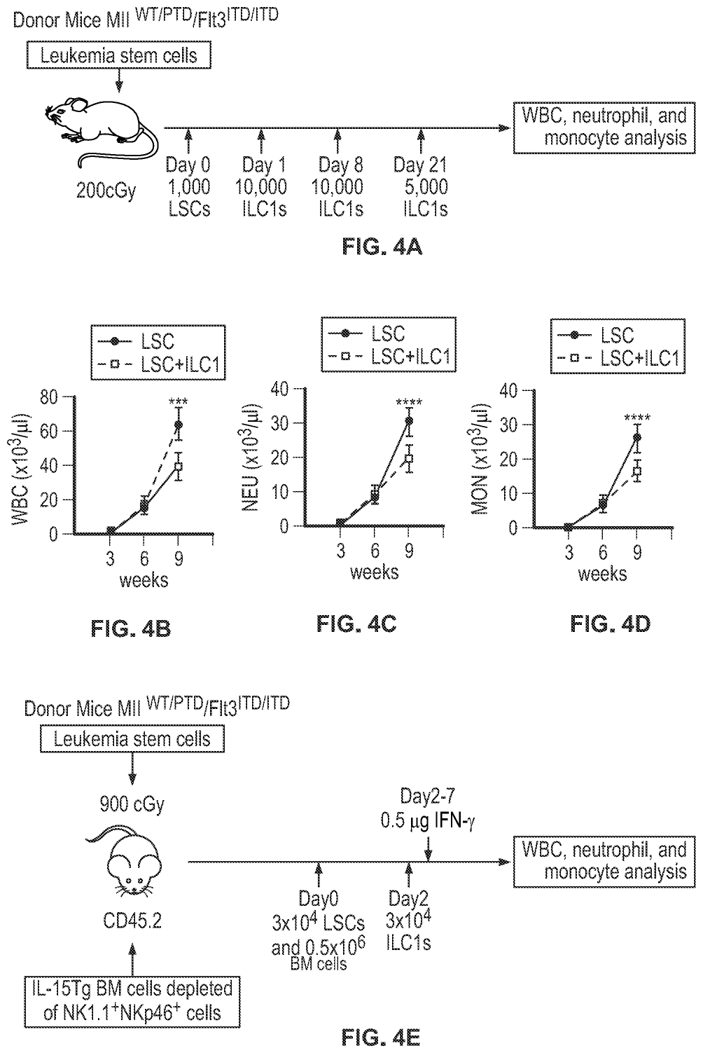

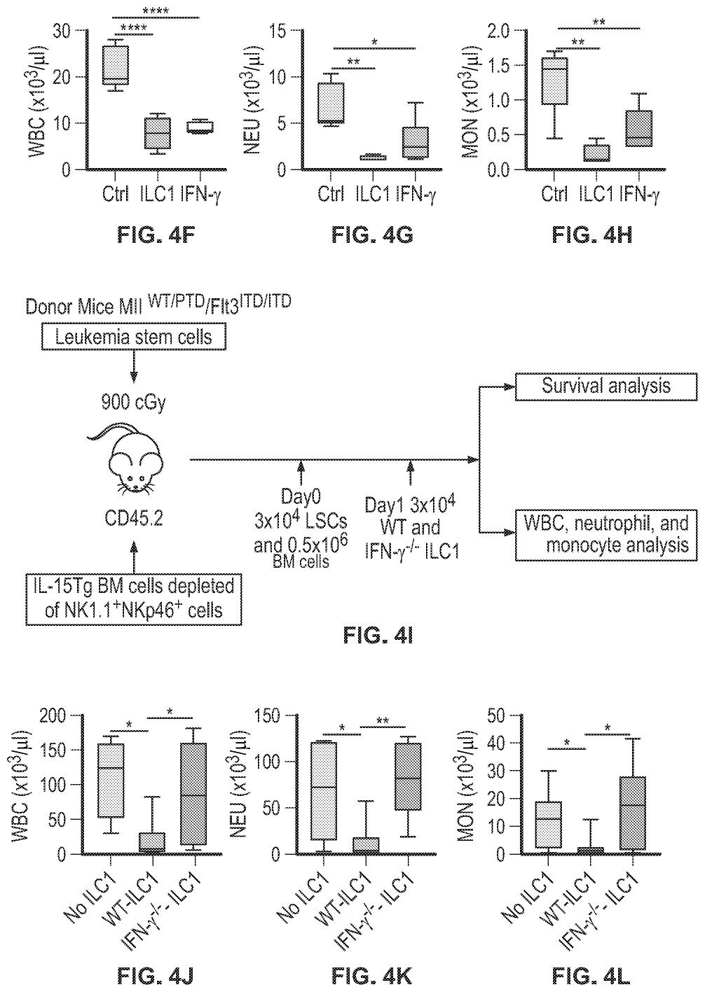

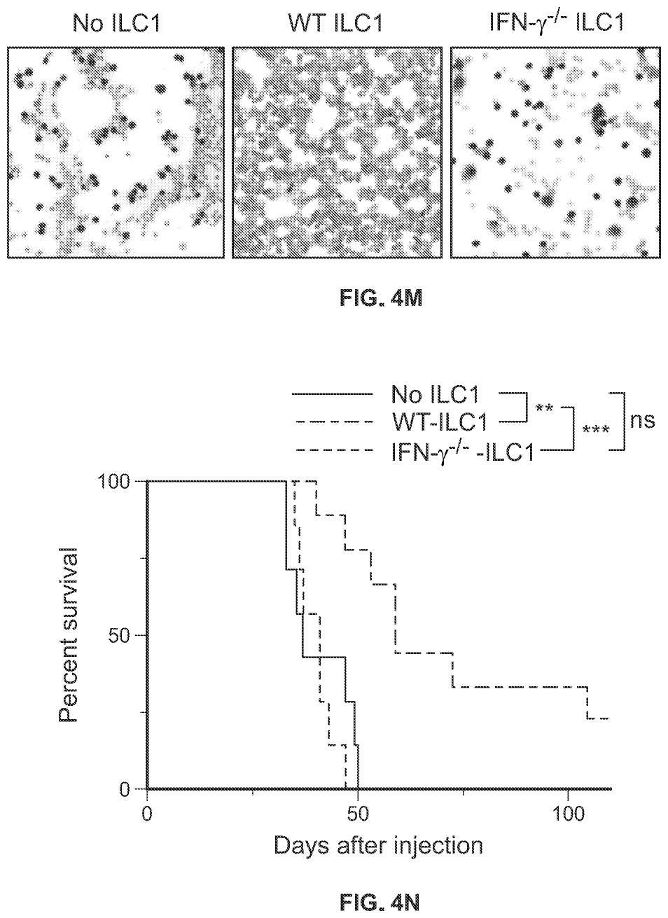

As shown in FIG. 4, ILC and ILC1-secreted IFN-γ control leukemia development and prolong the survival of leukemic mice Briefly, and a shown schematically in FIG. 4A, 3×10⁴ LSCs were intravenously injected into non-lethally irradiated (200 cGy) immunocompromised Rag2^{-/-}γc^{-/-} recipient mice on day 0. Mice were intravenously injected with 3×10⁴ mouse ILC from the livers of C57BL/6J (CD45.2) mice on day 1, 8, 15 and 22. Total white blood cells (WBCs; FIG. 4B), neutrophils (FIG. 4C), and monocytes (FIG. 4D) were measured at weeks 3, 6, 9 (n=9). (e) Schematic of the design and procedures for (FIG. 4F-H). Bone marrow cells were isolated from IL-15 transgenic mice and depleted of NK1.1⁺NKp46⁺ cells, and 3×10⁴ LSCs sorted from spleens of Mll^{PTD/WT:Flt}3^{ITD/ITD} mice were intravenously co-injected into lethally irradiated (900 cGy) C57BL/6J (CD45.2) recipient mice on day 0. Mice were intravenously injected with 3×10⁴ ILC or 0.5 μg recombinant murine IFN-γ on day 1. Total WBCs (FIG. 4f), neutrophils (FIG. 4g), and monocytes (FIG. 4h) were measured at week 5 (n=6). (FIG. 4I) As shown schematically in FIG. 4J, 3×10⁴ LSCs from the spleens of Mll^{PTD/WT}:Flt3^{ITD/ITD} mice were intravenously injected into lethally irradiated (900 cGy) C57BL/6J (CD45.2) recipient mice on day 0. Mice were intravenously injected with 3×10⁴ mouse ILC from the livers of WT or IFN-γ^{-/-} mice on day 1. Total WBCs (FIG. 4J), neutrophils (FIG. 4K), and monocytes (FIG. 4L) were measured at week 6 (n=5-8). (FIG. 4M) Images of peripheral blood smears are shown (n≥3 for each group). (FIG. 4N) Kaplan-Meier survival analysis for mice injected with or without WT ILC1, or IFN-γ^{-/-} ILC1s by Kaplan-Meier method and log-rank test (n=7-9). All non-survival data are shown as mean±SD. P values were calculated by either one-way ANOVA models or linear mixed models. *p<0.05, **p<0.01, ****p<0.0001, ns, not significant.

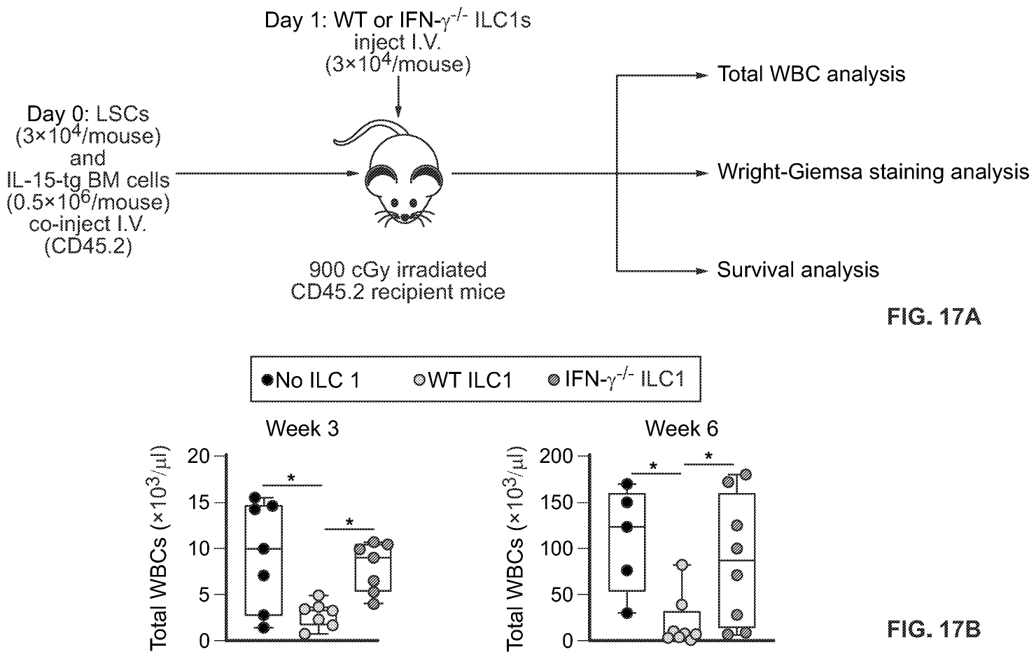

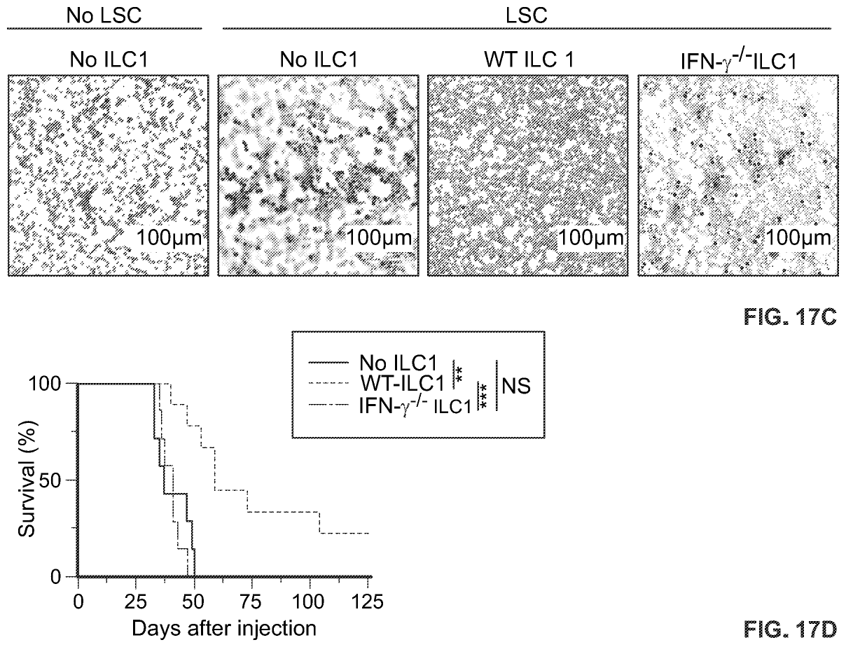

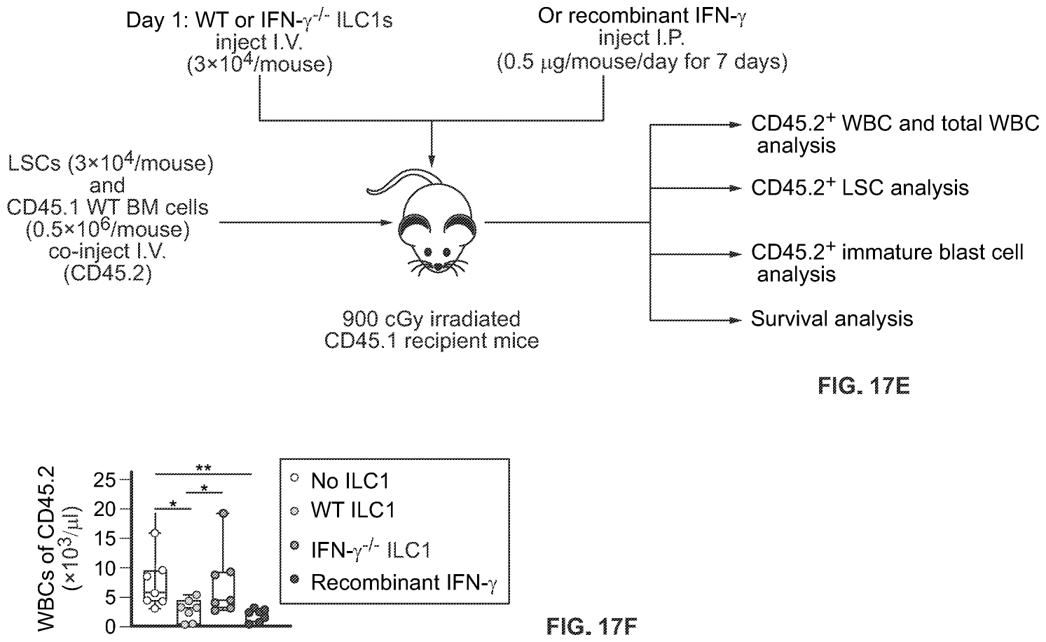

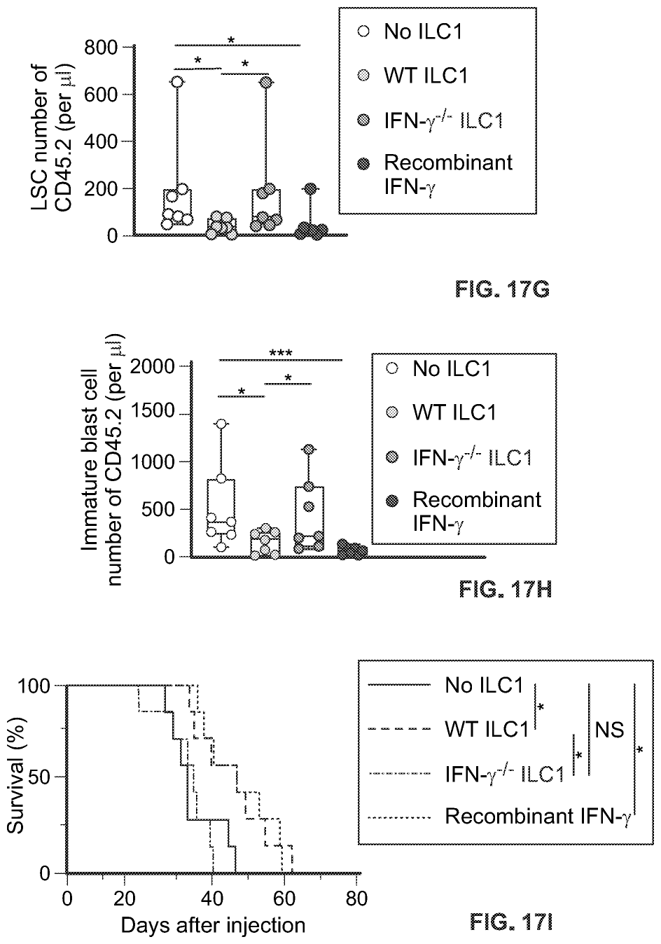

Experiments were designed to test whether ILC1s could suppress leukemia development and growth in vivo. When we initiated the in vivo efficacy experiment, we did not know whether ILC could survive well in vivo after their adoptive transfer. Since IL-15 is a critical cytokine that supports the survival of ILC1s^{35-36,63}, we first tested whether adoptively transferred WT ILC1s can suppress the development of leukemia derived from LSCs when bone marrow cells from IL-15 transgenic (IL-15tg) mice³⁴ were co-injected as support cells into recipient mice pre-integrated with LSCs (FIG. 17A). In this model, we observed that mice injected with WT ILC1s had significantly fewer total WBCs when compared to IFN-γ^{-/-} ILC1s and untreated groups, the latter of two which did not differ significantly from each other (FIG. 17B). Additionally, in this model, a substantial reduction of the immature blast cell population in the blood (detected with Giemsa staining) and significantly prolonged survival were also observed in mice injected with WT ILC1s compared to untreated mice or those treated with IFN-γ^{-/-} ILC1s (FIGS. 17C-17D). Using recombinant murine IFN-γ to replace WT ILC1s in this experiment, an effect similar to that of WT ILC1s was observed, that is, the recombinant IFN-γ had significantly fewer total WBCs when compared to untreated group (FIGS. 27A-27C).

However, the above model could not distinguish WBCs derived from LSCs and those derived from transplanted normal donor bone marrow. Therefore, we utilized CD45.1 and CD45.2 congenic mice to further test our hypothesis. In this congenic mouse model, we sorted CD45.2 LSCs from Mll^{PTD/WT}: Flt3^{ITD/ITD} mice with AML, and co-injected them along with CD45.1⁺ bone marrow cells as support cells into lethally irradiated CD45.1 recipient mice. The next day, we injected WT ILC or IFN-γ^{-/-} ILC i.v. or recombinant IFN-γ cytokine intraperitoneally (i.p.) (FIG. 17E). Total WBCs (CD45.1⁺ and CD45.2⁺ WBCs), CD45.2⁺ WBCs, CD45.2⁺ LSCs, and CD45.2⁺ immature blast cells (which have been reported to accumulate in AML 69) were counted 3 weeks post LSC implantation (FIGS. 17E-17H). Flow cytometry analysis confirmed that donor and host cells could be distinguished using anti-CD45.2 and anti-CD45.1 antibodies (FIG. 27B). In this model with regular bone marrow instead of IL-15tg mouse bone marrow as support cells, we also observed that compared to untreated or IFN-γ^{-/-} ILC1s-treated mice, mice treated with WT ILC or recombinant IFN-γ had a significantly reduced CD45.2⁺ WBC and total WBC in PB (FIG. 17F and FIG. 27C) and possessed significantly fewer donor-derived LSCs and immature blasts (FIGS. 17G-17H). The treated mice also survived significantly longer than the untreated or IFN-γ^{-/-} ILC1-treated mice (FIG. 17I). The results indicate that ILC1s and IFN-γ derived from them are sufficient to suppress leukemogenesis in vivo.

### Example 6: Identification of the LSC Regulatory Pathways Exploited by ILC1s or ILC1 Secreted IFN-γ

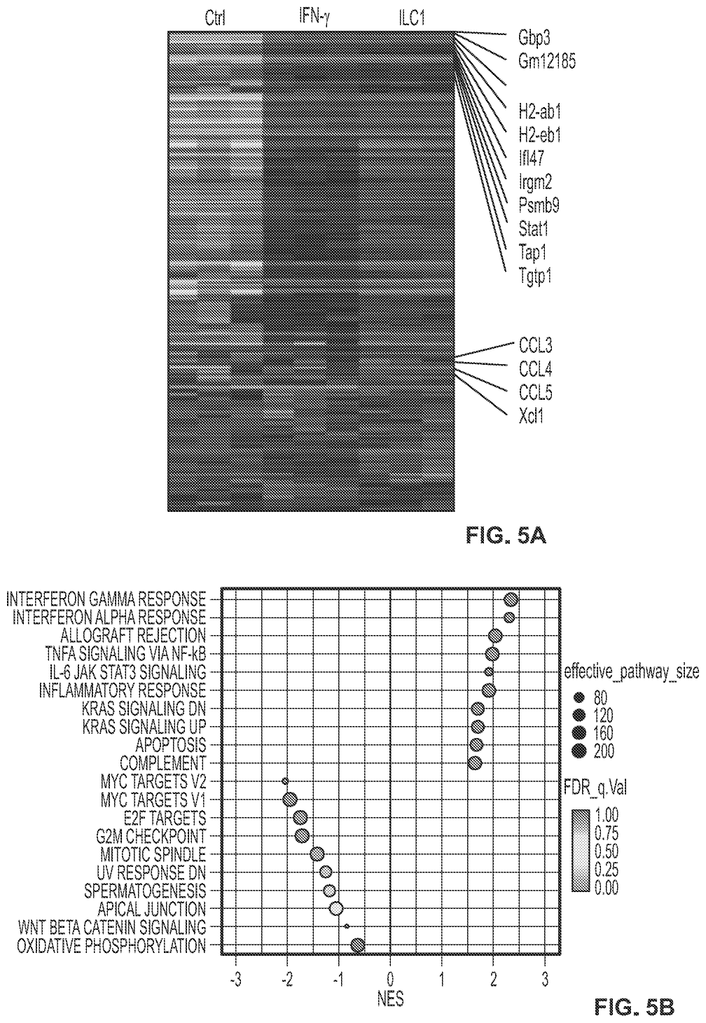

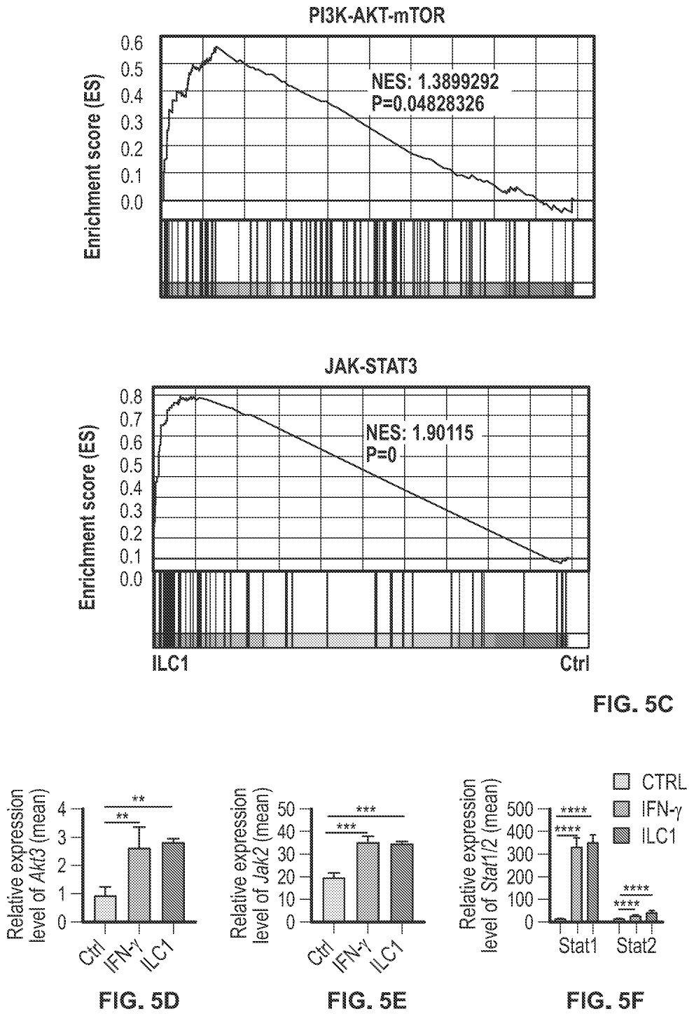

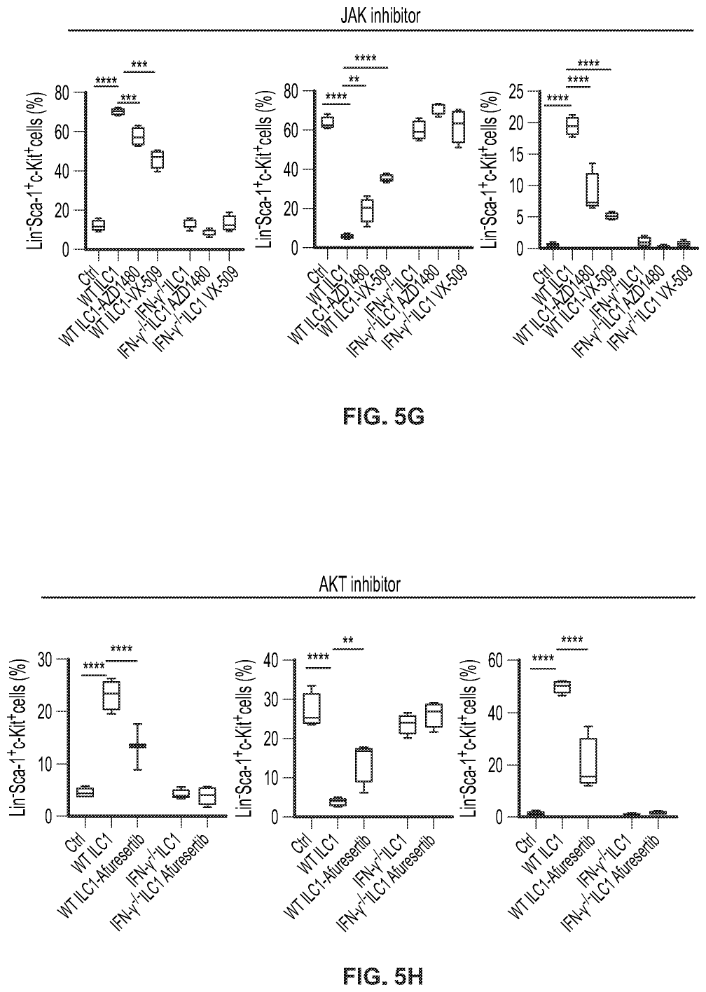

To investigate the mechanisms by which ILC1 and ILC1-secreted IFN-γ regulate LSCs, Ribozero RNA-seq analysis was performed on LSCs co-cultured with or without ILC1s isolated or treated with recombinant murine IFN-65 . Following the ILC1—LSC co-culture, the LSCs from separated from the ILC1s using FACS prior to RNA-seq analysis. RNA-seq revealed that 445 and 93 LSC genes were significantly up- and downregulated, respectively, following co-culture with ILC1s as compared to LSC alone (FIG. 5A). RNA-seq also revealed 320 and 82 LSC genes were up- and downregulated, respectively, following treatment with recombinant IFN-γ (FIG. 5A). Furthermore, a large number of up- and downregulated genes overlapped between LSCs co-cultured with ILC1s and LSCs treated with IFN-γ compared to LSC alone (FIG. 5A). Interestingly, among upregulated genes unique to the ILC1 co-culture, 3 out of the top 10 were chemokines (Ccl3, Ccl4, and Xcl1; FIG. 11A). These data suggest that the interaction of ILC1s with LSCs results in the recruitment of additional immune cells into the tumor microenvironment (TME) to control the development of AML.

Gene set enrichment analysis (GSEA) was used to identify the top 10 pathways in which those upregulated and downregulated genes were enriched (FIG. 5B). LSCs co-cultured with ILC1s or treated with IFN-γ showed activated apoptotic pathways in the LSCs, while causing significant suppression of E2F targets, G2M checkpoints, MYC targets, and mitotic spindle pathways (FIG. 5B, FIGS. 11B-11D). Additionally, upon being co-cultured with ILC1s or treated with IFN-γ, LSCs showed activation of the JAK-STAT and PI3K-AKT signaling pathways (FIGS. 5C-5F, FIGS. 11E-11F). The LSCs co-cultured with ILC1s or treated with IFN-γ also showed increased expression of Akt3, Jak2, Stat1/2, Irf1/2/7/8/9, and suppressor of cytokine signaling 1 (Socs1), all of which are downstream of the IFN-γ signaling pathway^{38,39} (FIGS. 5C-5F, FIGS. 11E-11F). This indicates that ILC1s regulate LSCs via their secretion of IFN-γ and suggests that the ILC1s or their secreted IFN-γ utilize the JAK-STAT and PI3K-AKT signaling pathways to regulate LSCs. To further investigate this, LSCs were pretreated for 30 min with inhibitors of signaling components involved in these two pathways, prior to co-culture with WT ILC or IFN-γ^{-/-} ILC1s. The JAK2 inhibitor AZD1480 and the JAK1/2/3 inhibitor VX-509 significantly suppressed the observed ILC1-mediated reduction of LSC differentiation into LS⁻K⁺ leukemic progenitor cells and the observed ILC1-mediated induction of LSC differentiation into non-leukemic LS⁺K⁻ cells (FIG. 5G). The number of LSCs also decreased in the presence of AZD1480 or VX-509 when compared to the corresponding control (FIG. 5G). However, the two JAK inhibitors had no effect on LSCs co-cultured with IFN-γ^{-/-} LSCs. Similar results were seen using the AKT inhibitor, afureserertib (FIG. 5H). These data suggest that ILC1s and ILC1-derived IFN-γ regulate the differentiation of LSCs though JAK-STAT and PI3K-AKT signaling pathways.

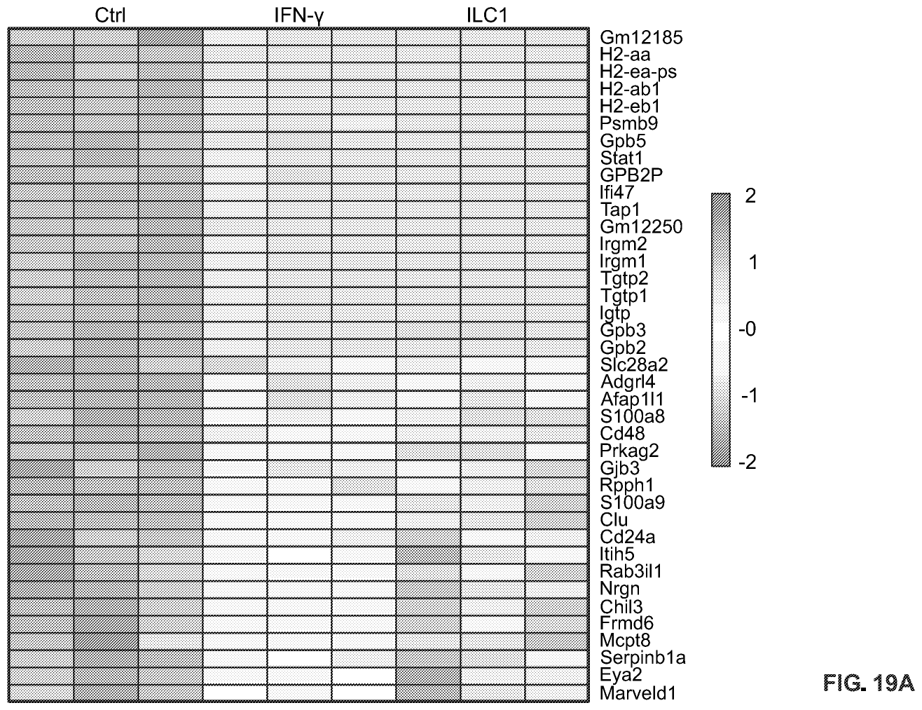

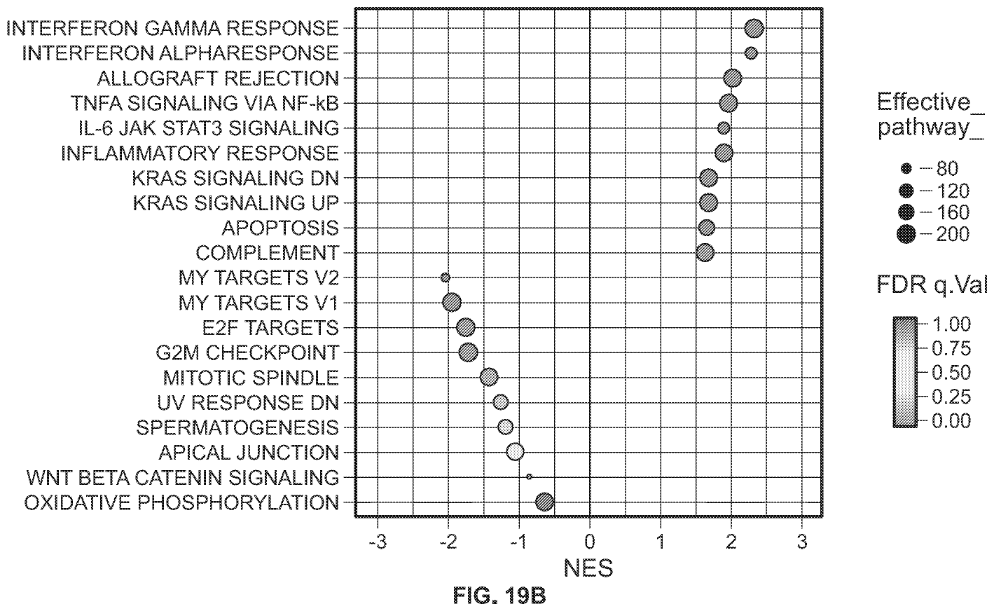

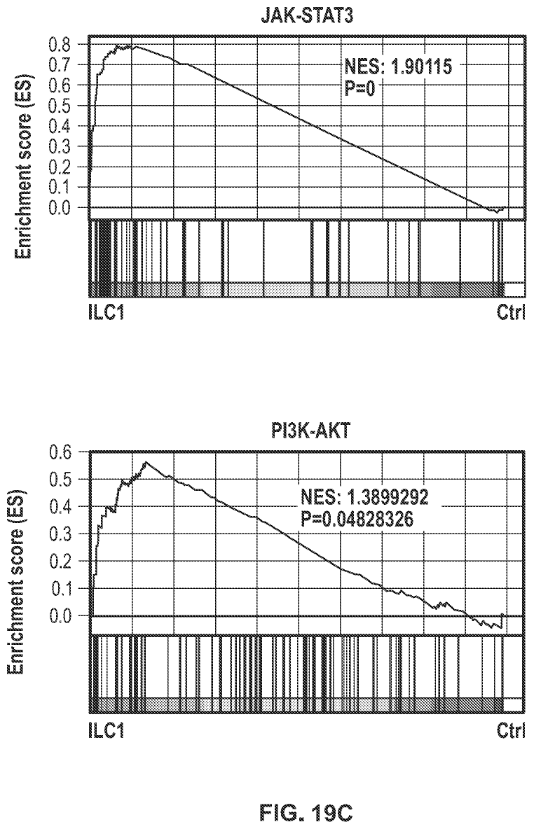

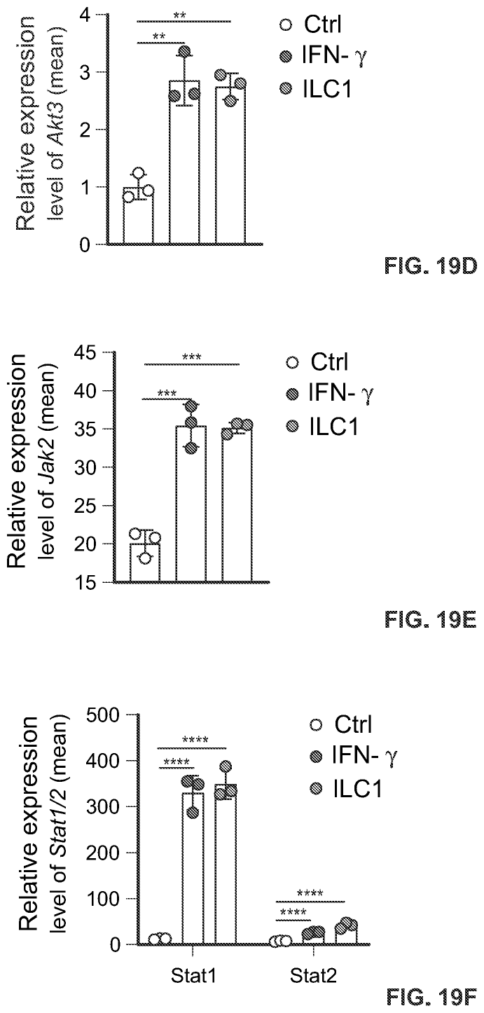

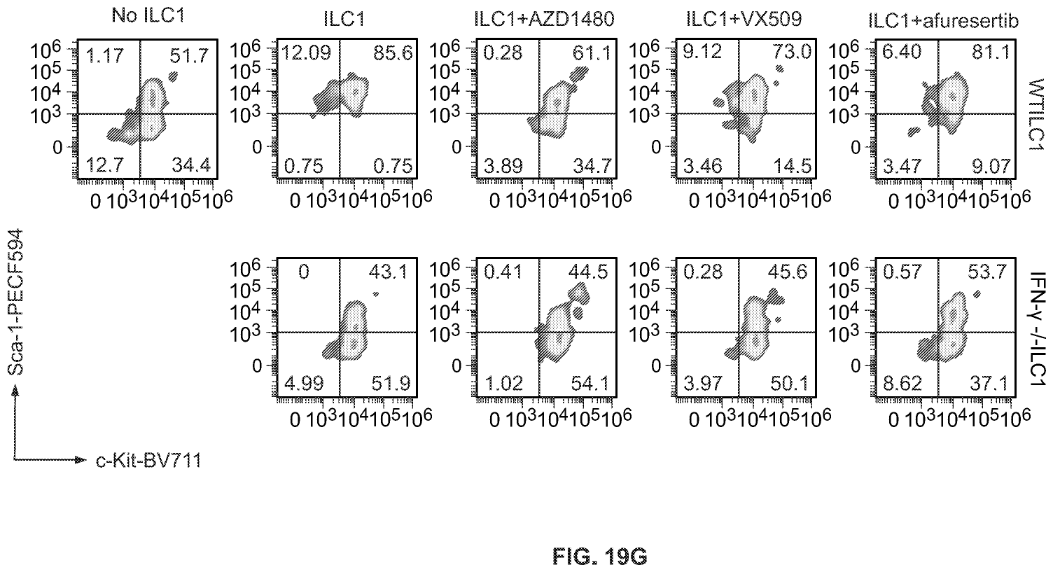

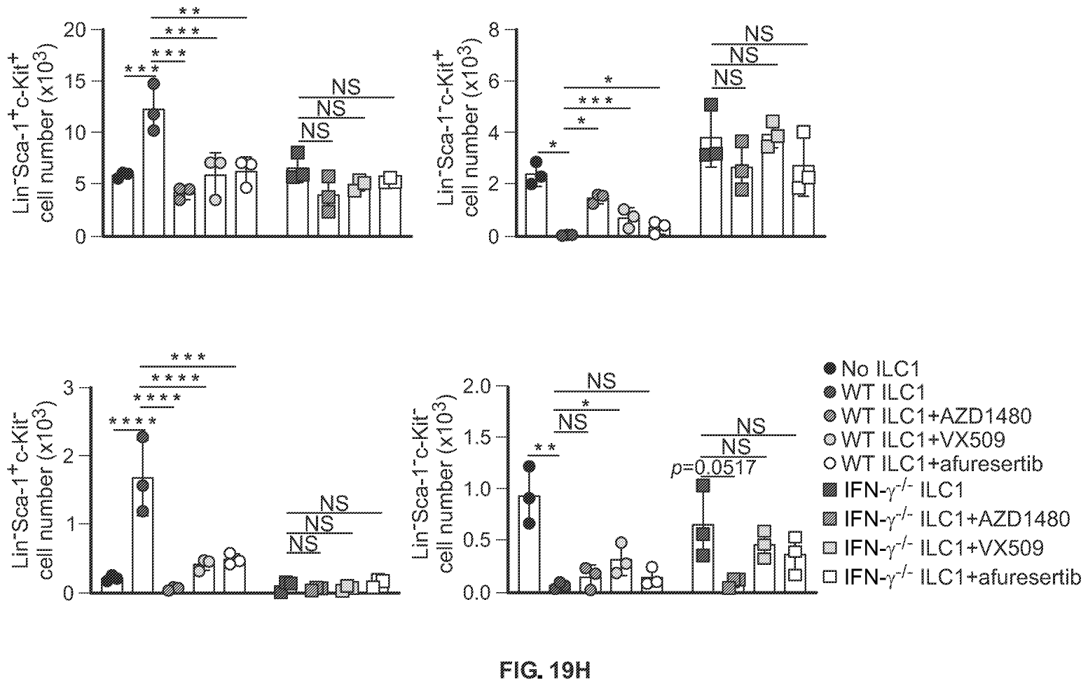

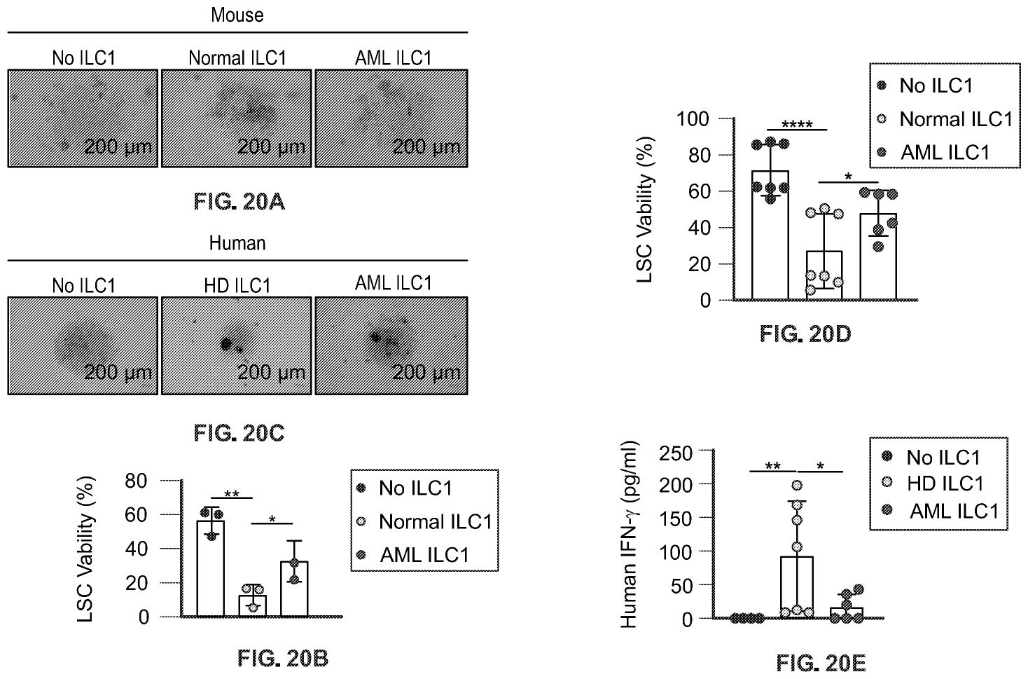

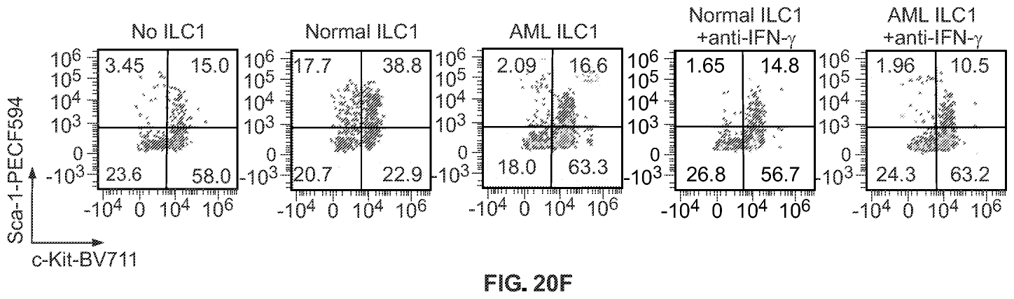

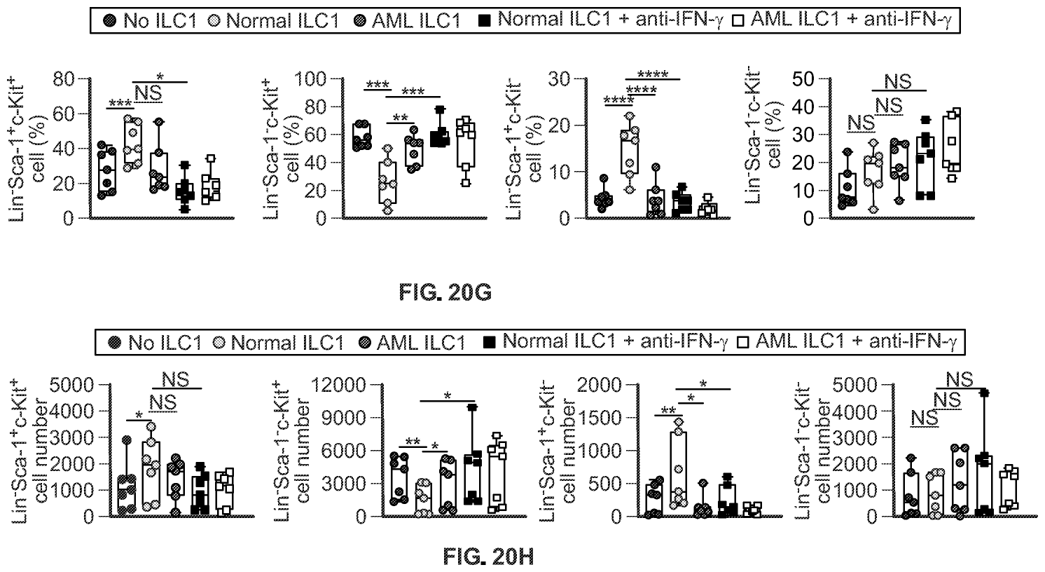

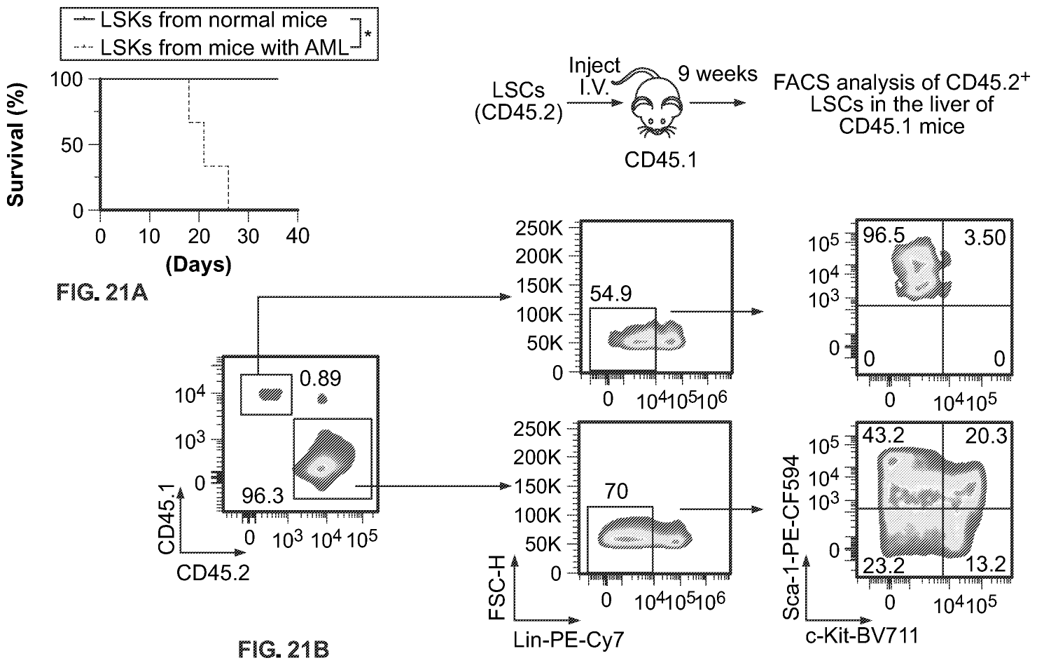

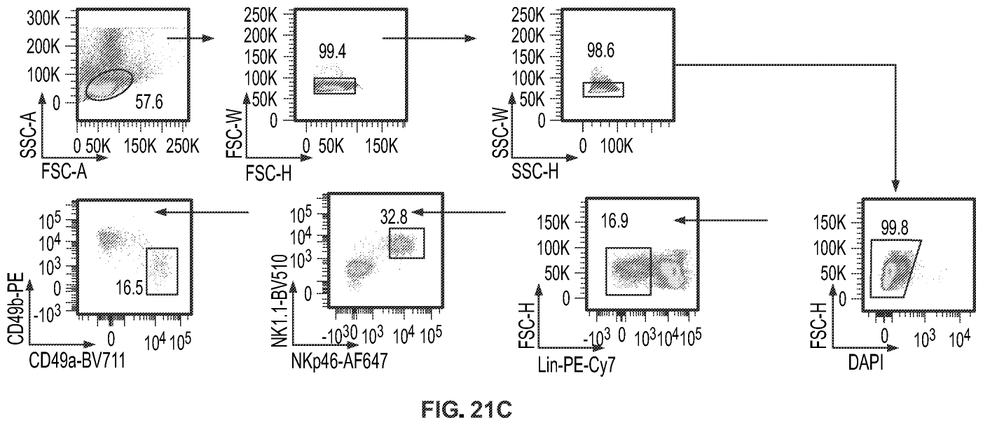

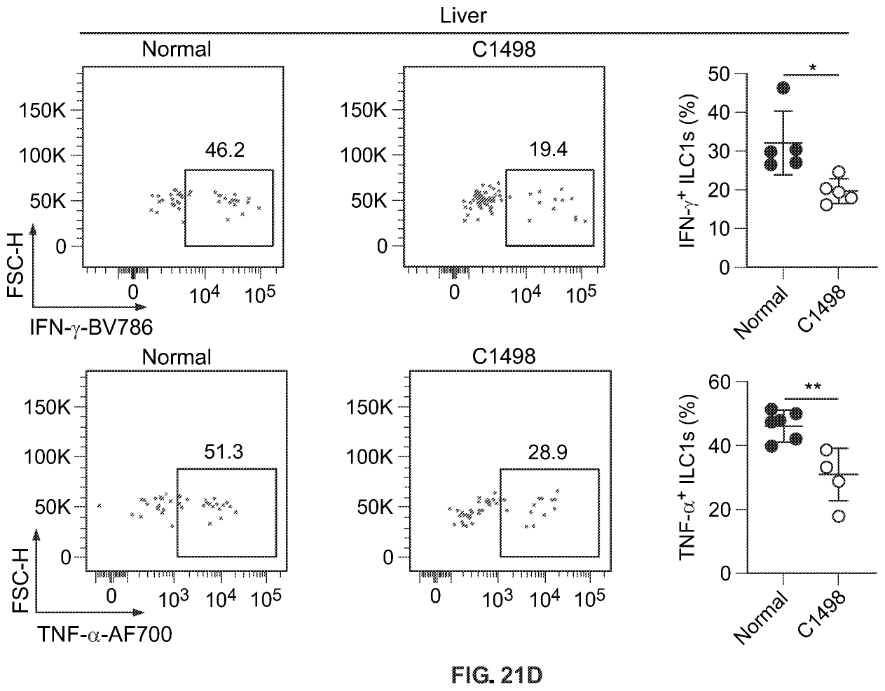

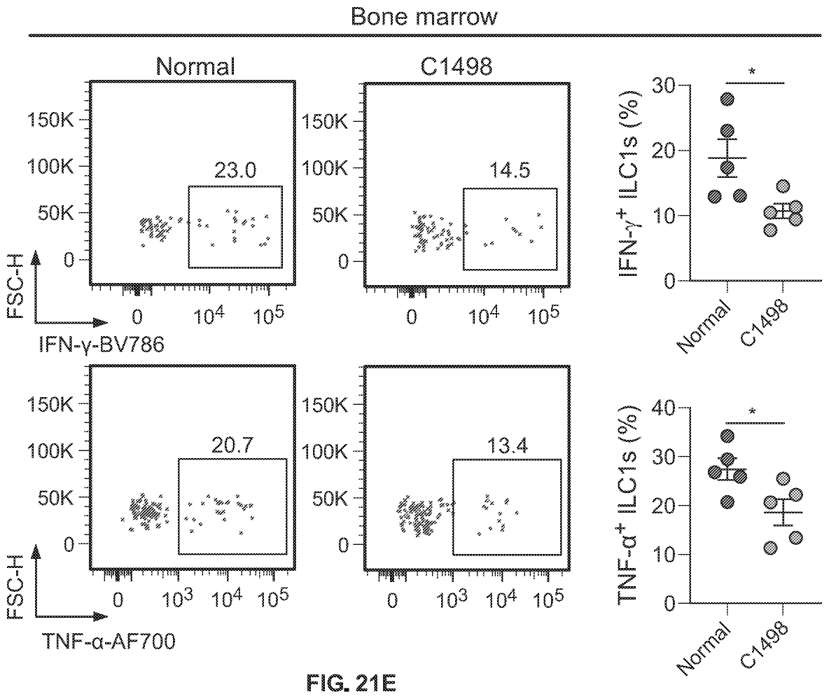

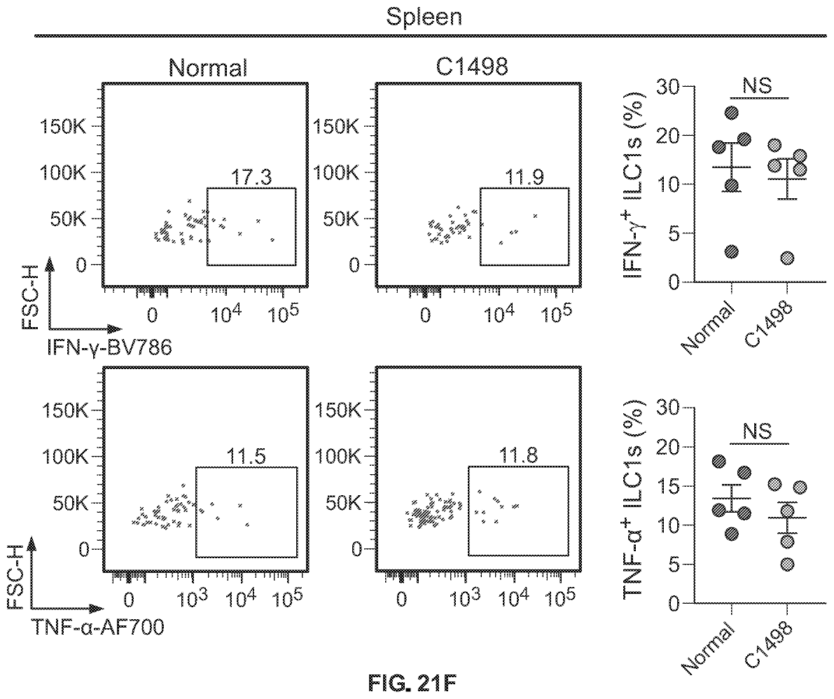

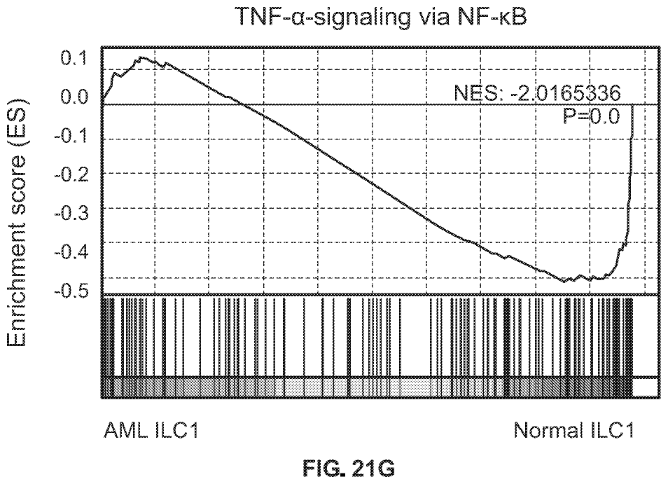

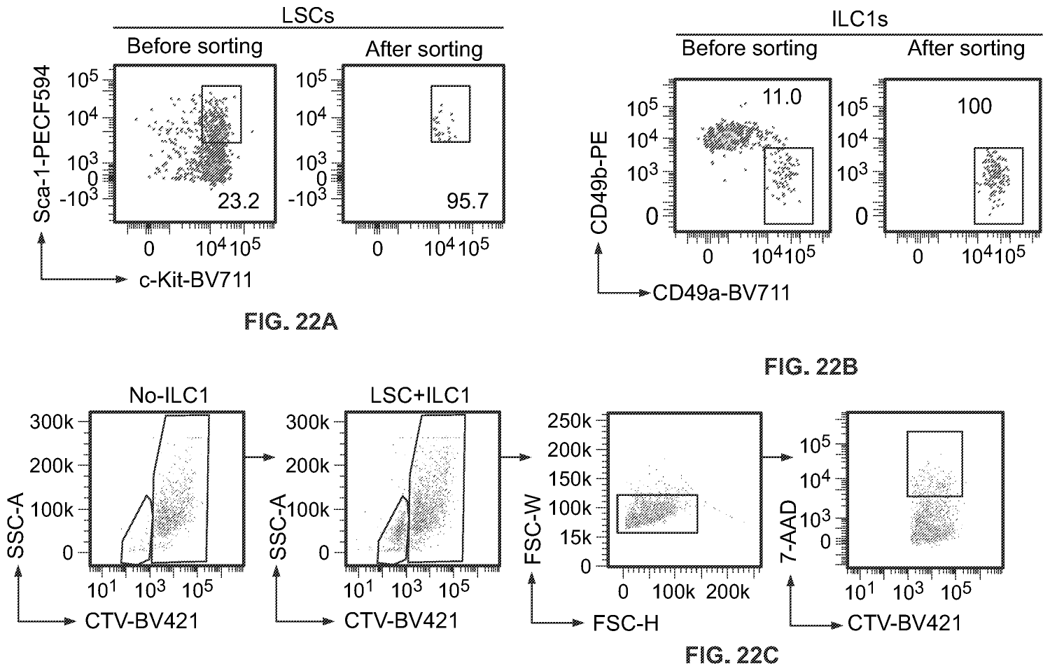

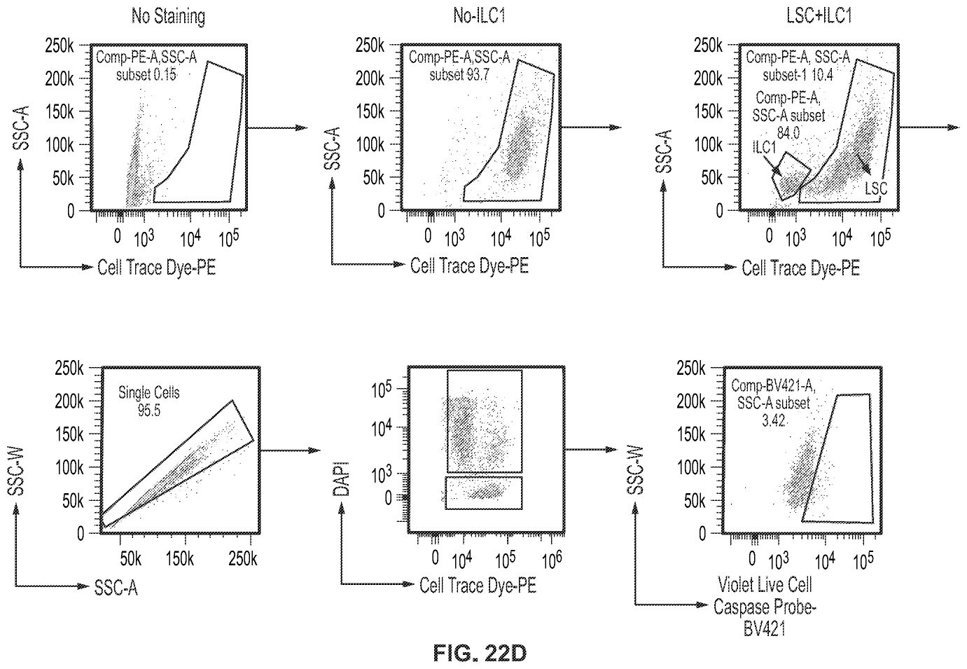

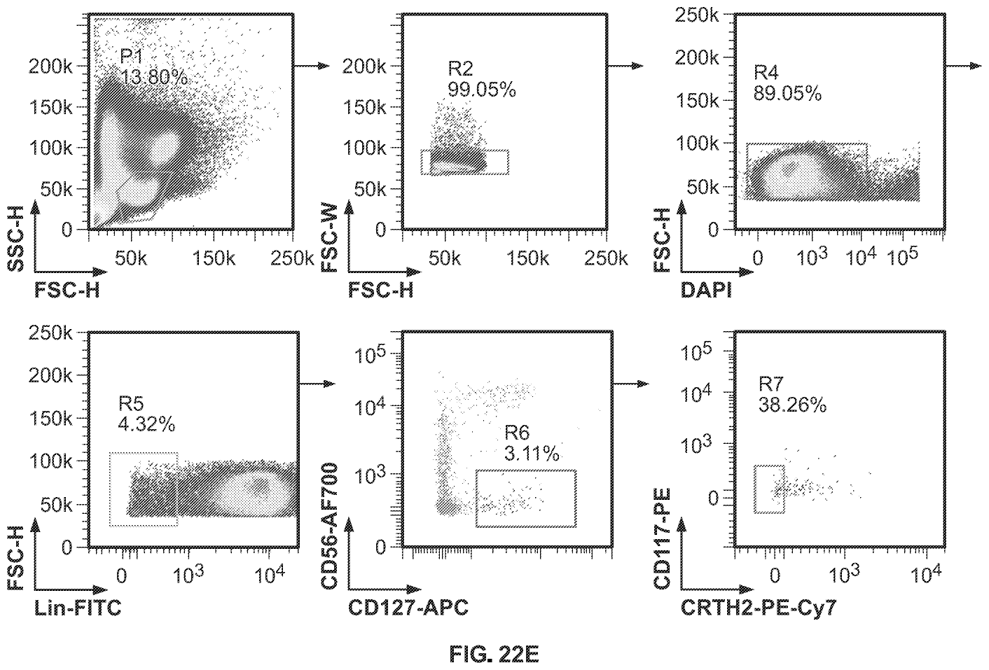

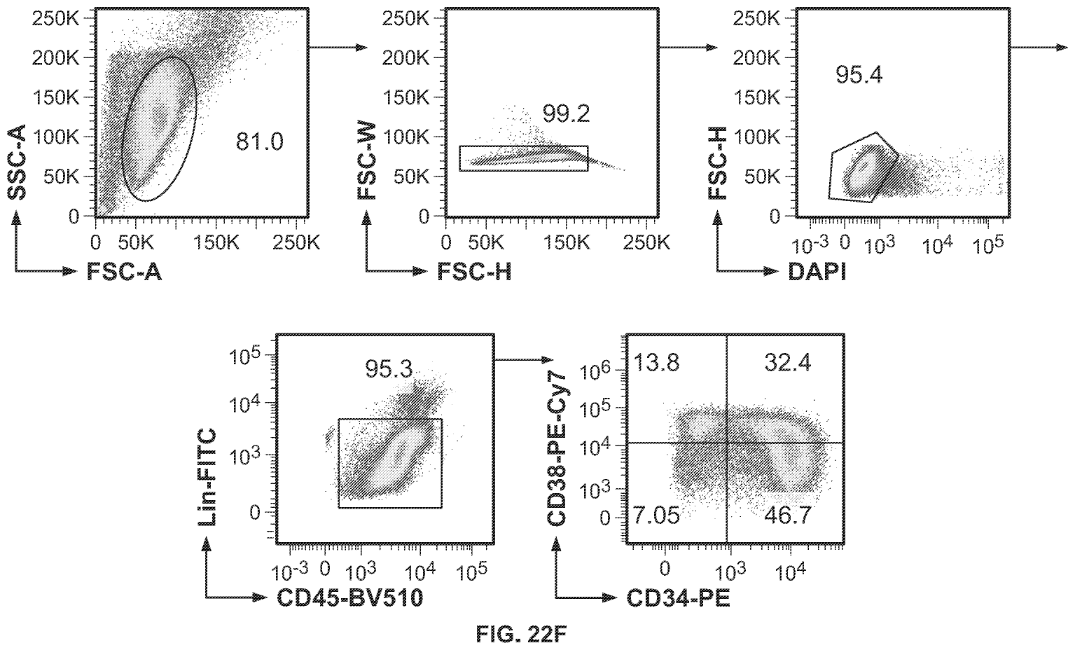

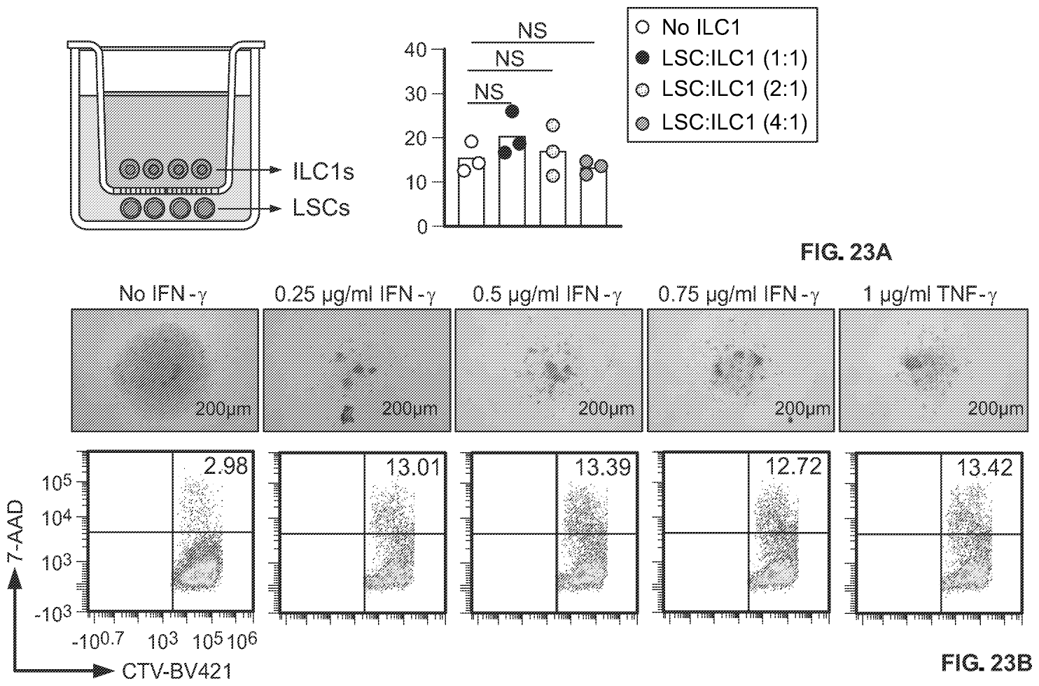

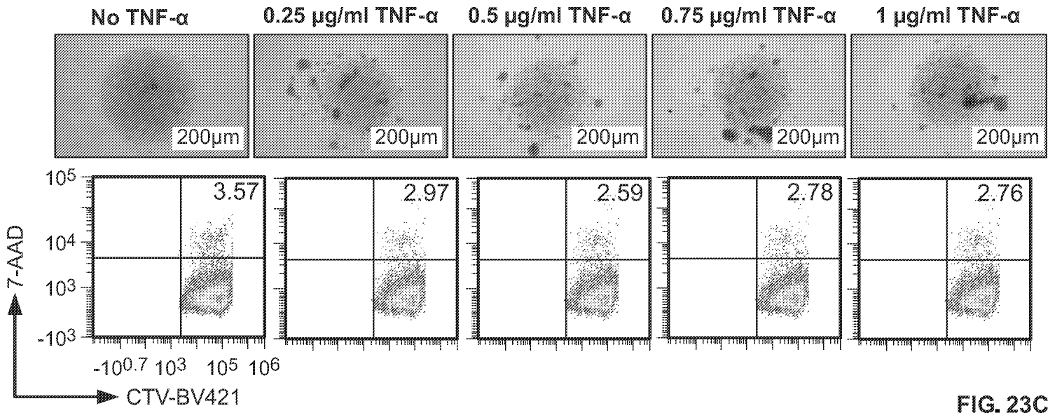

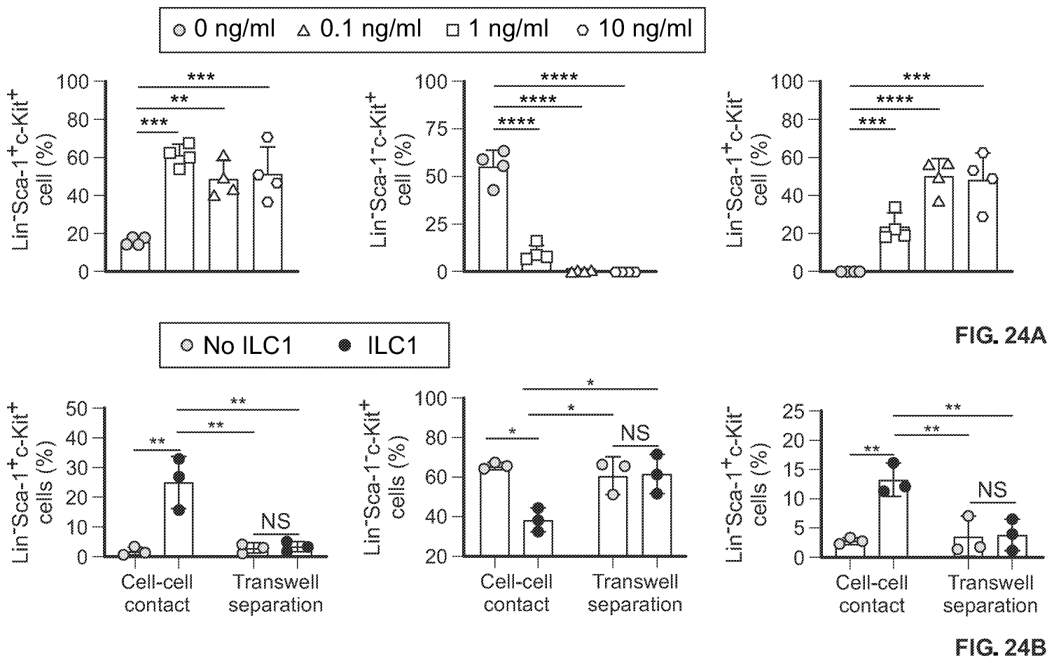

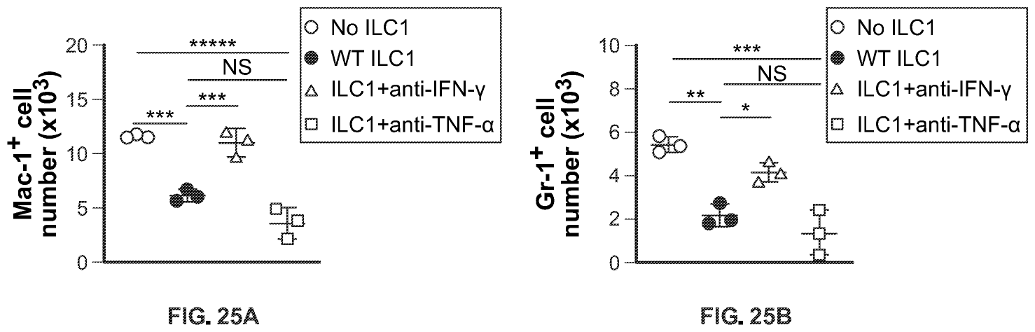

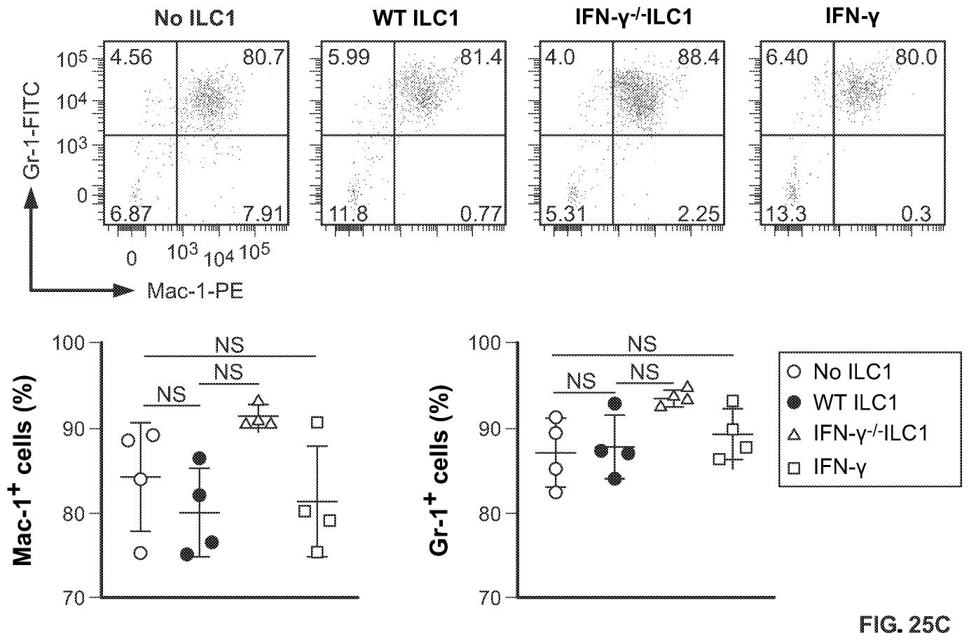

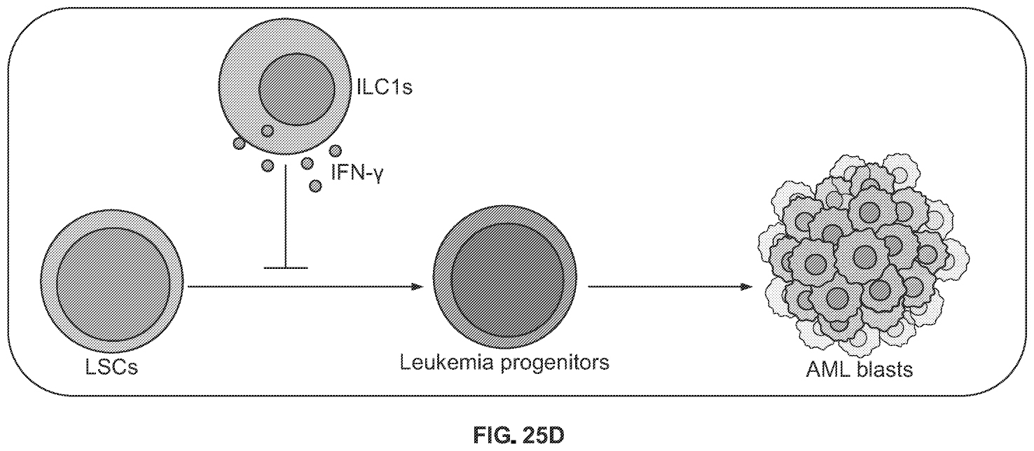

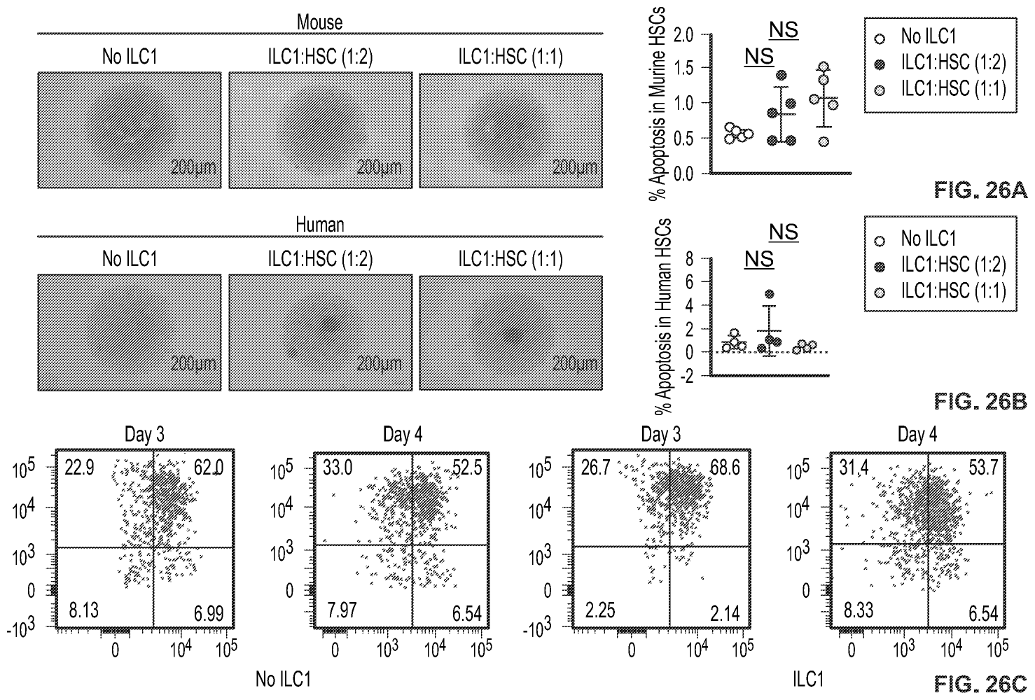

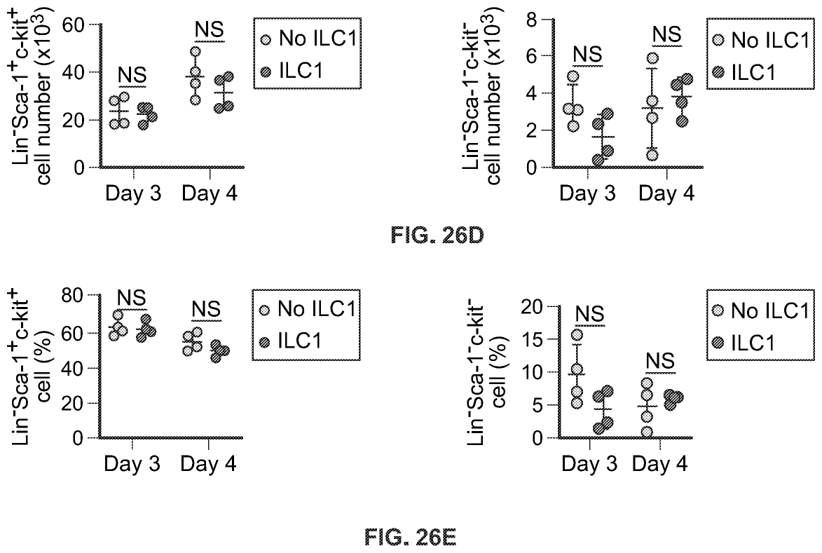

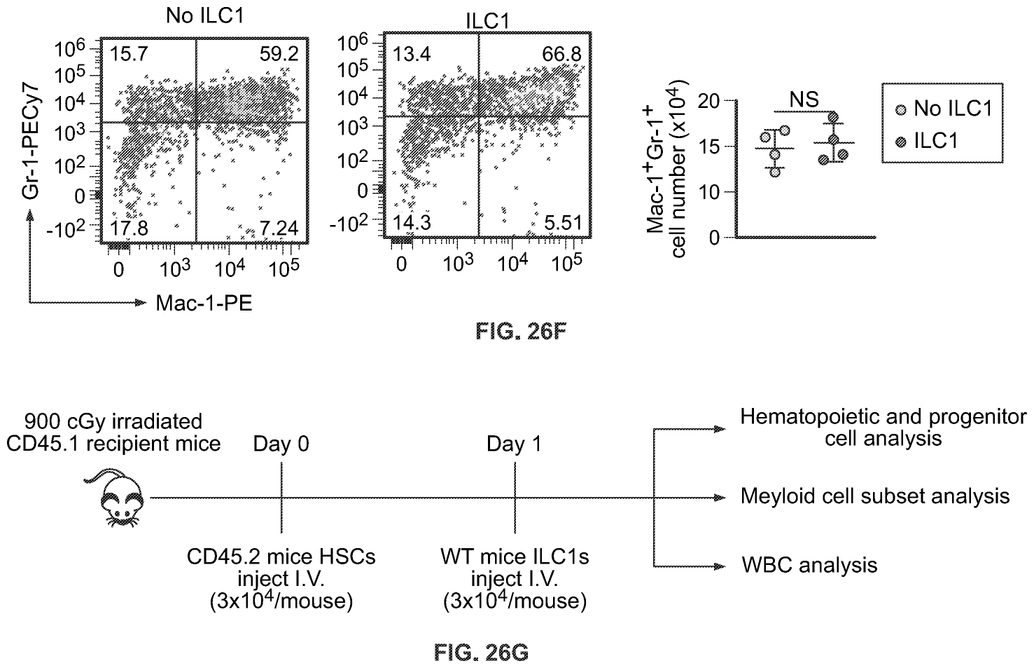

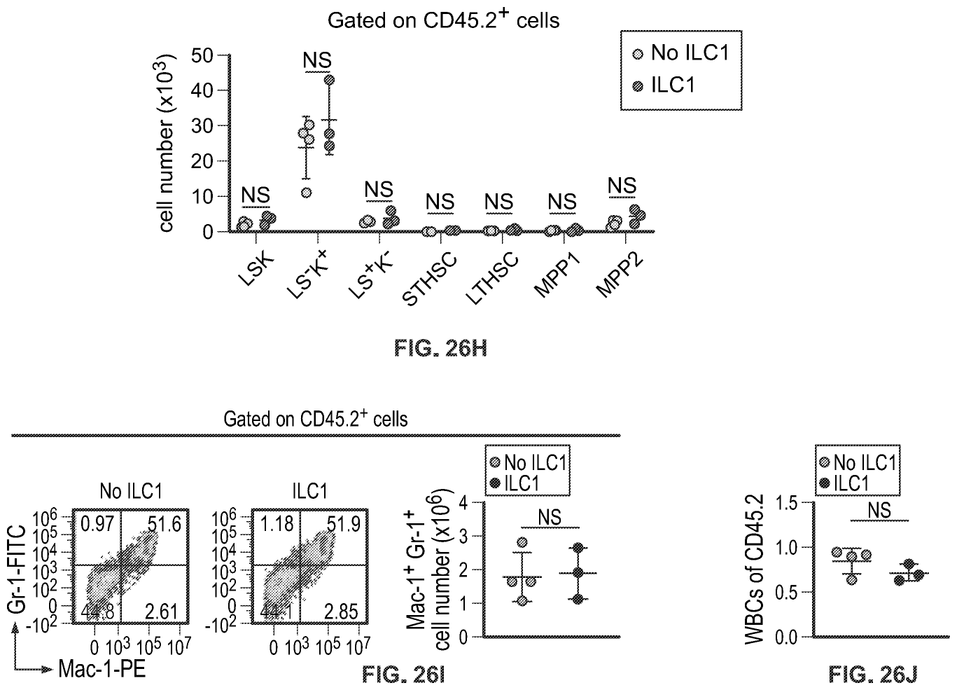

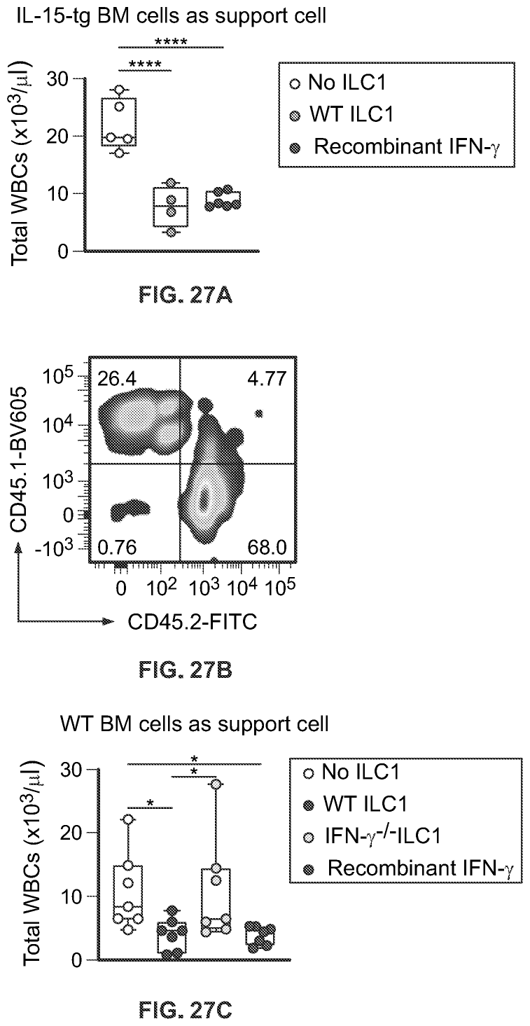

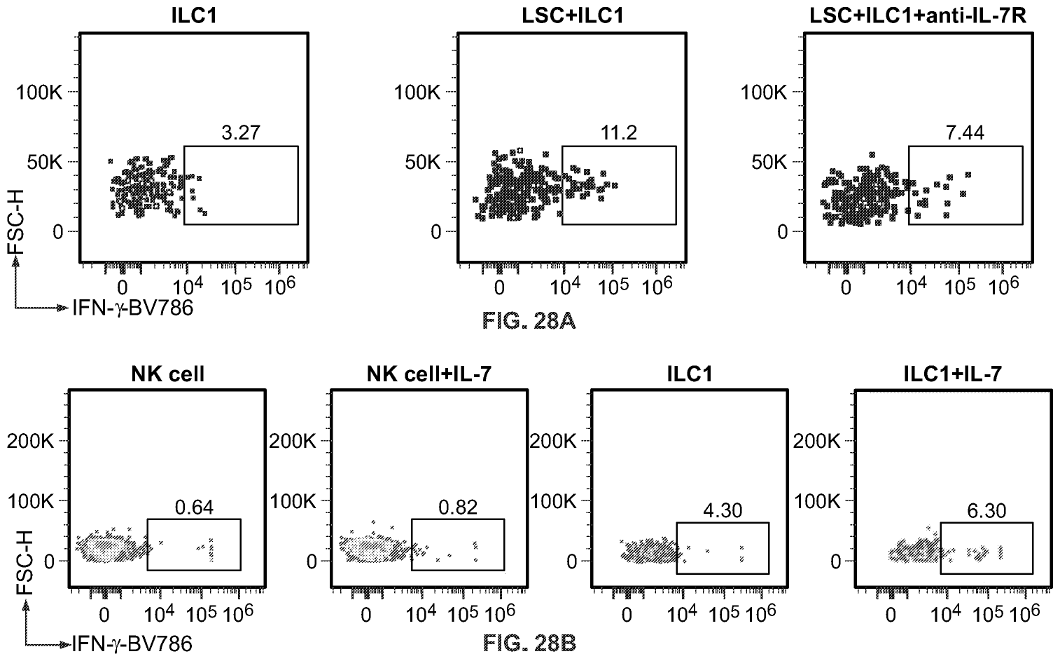

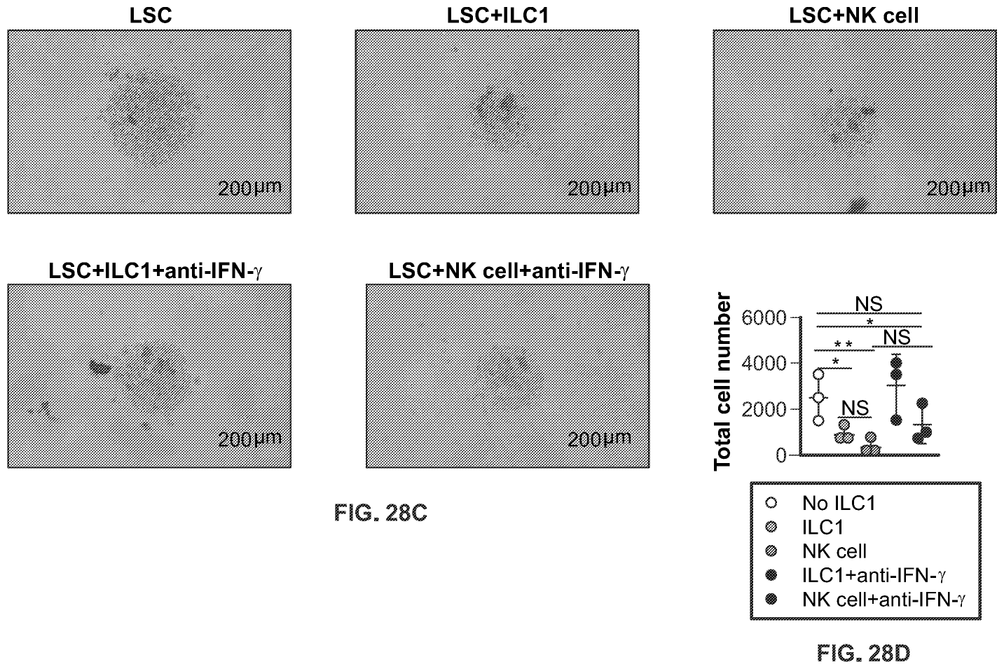

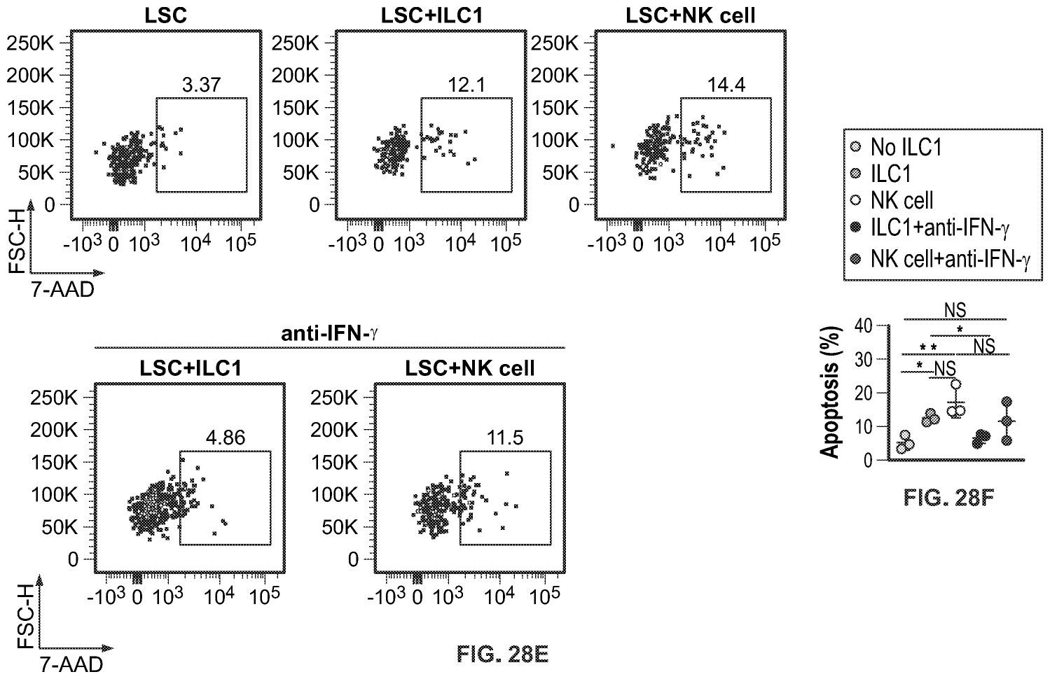

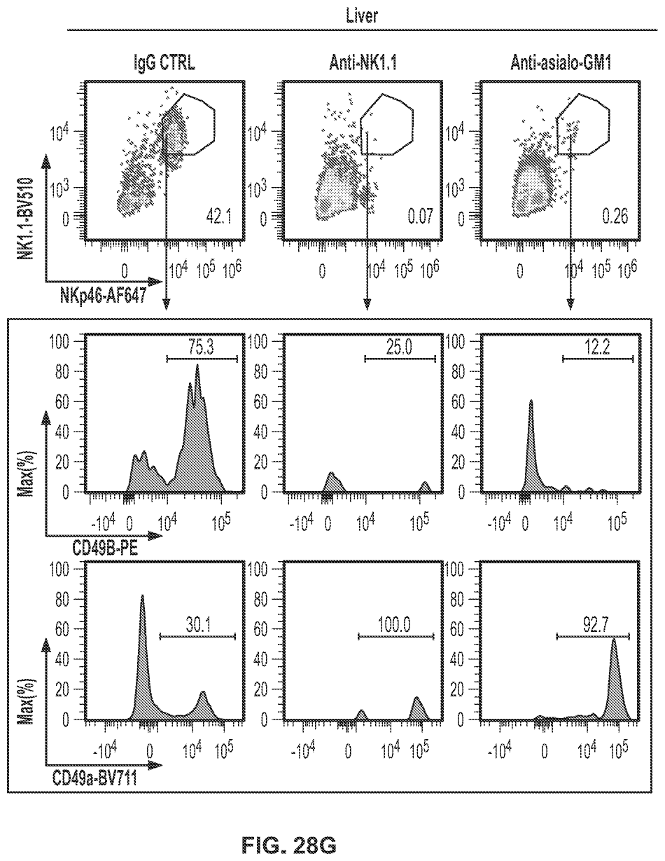

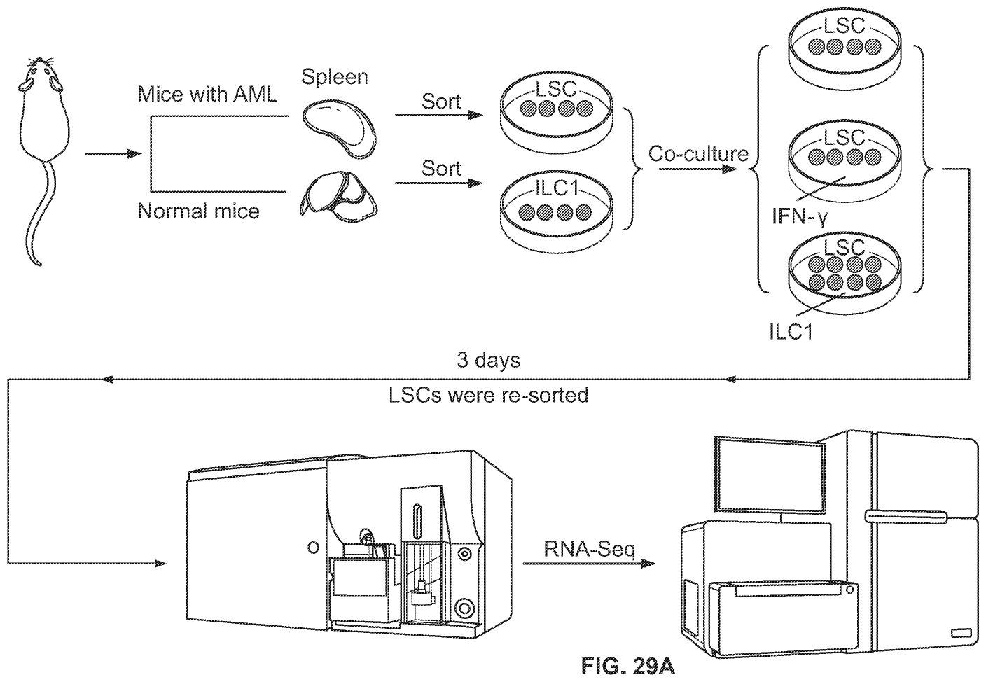

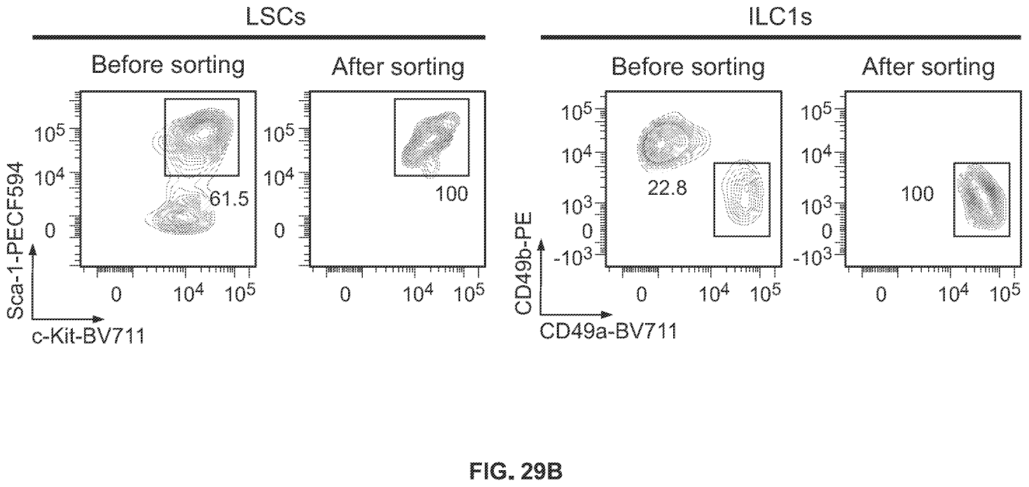

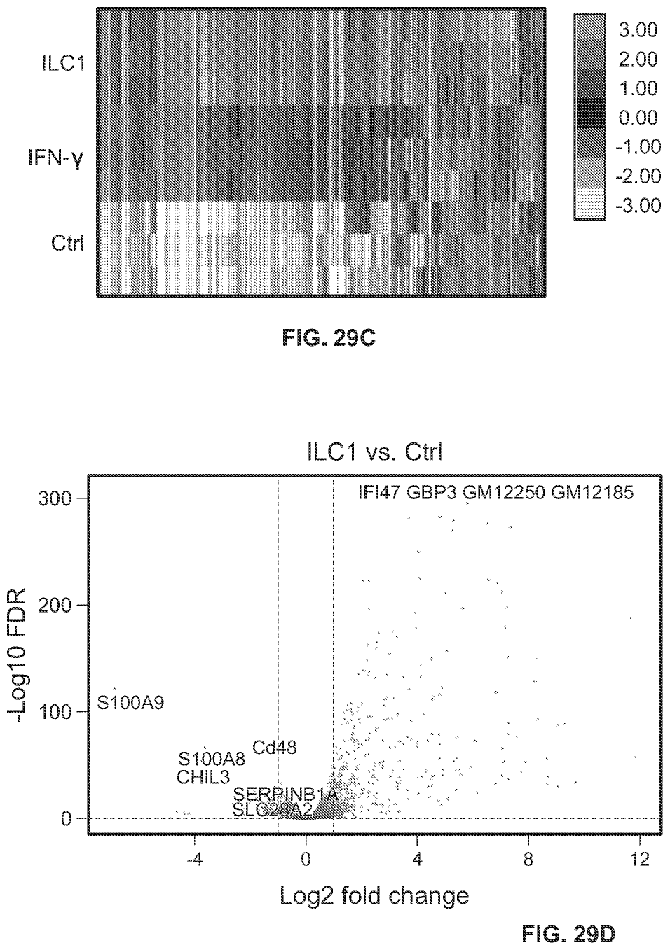

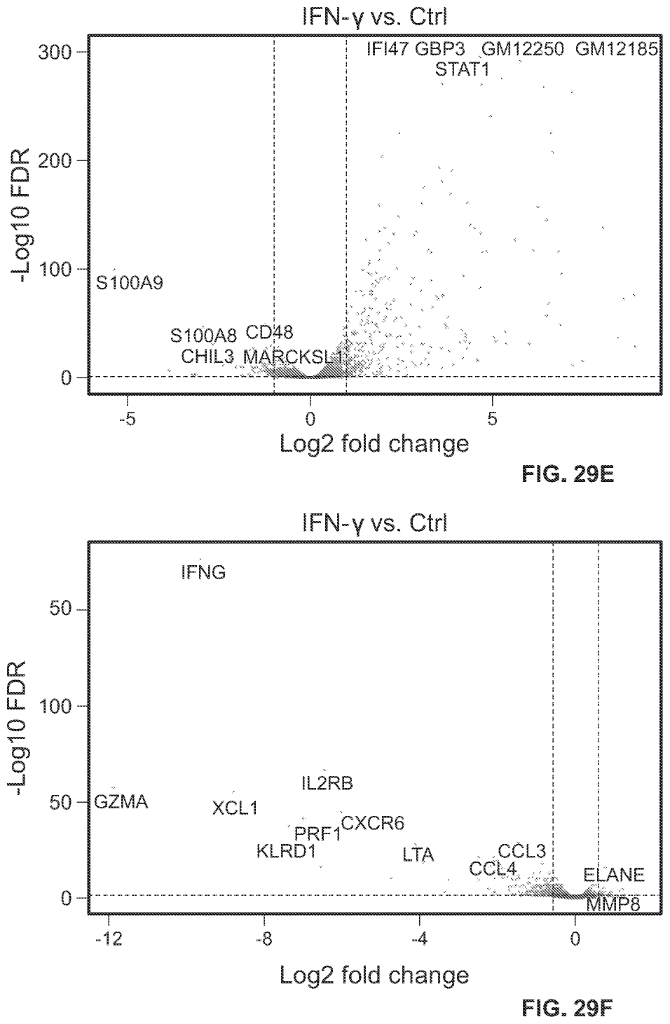

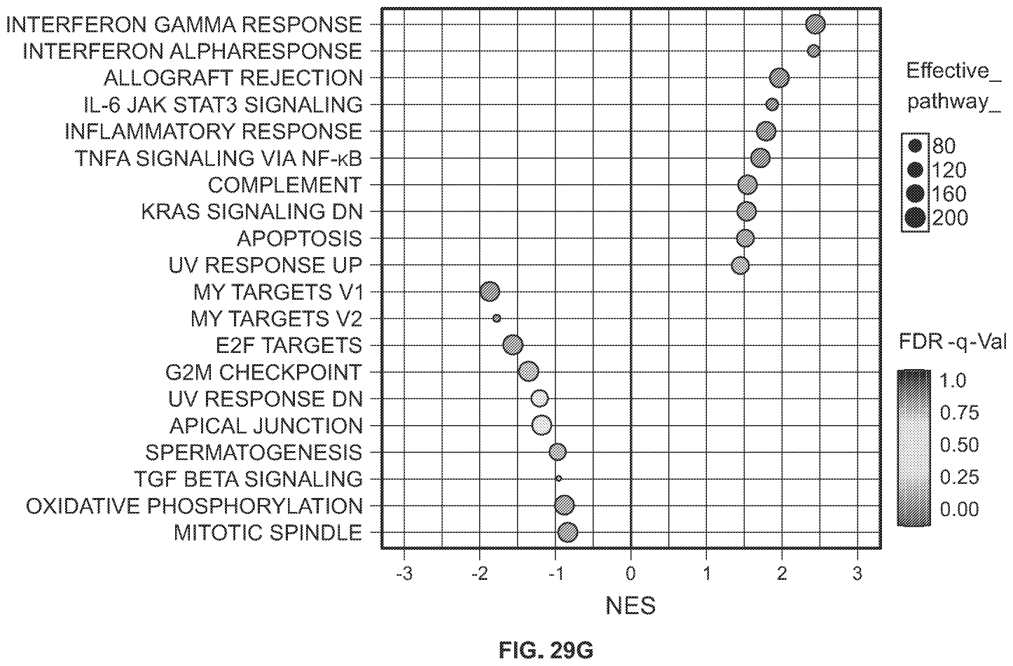

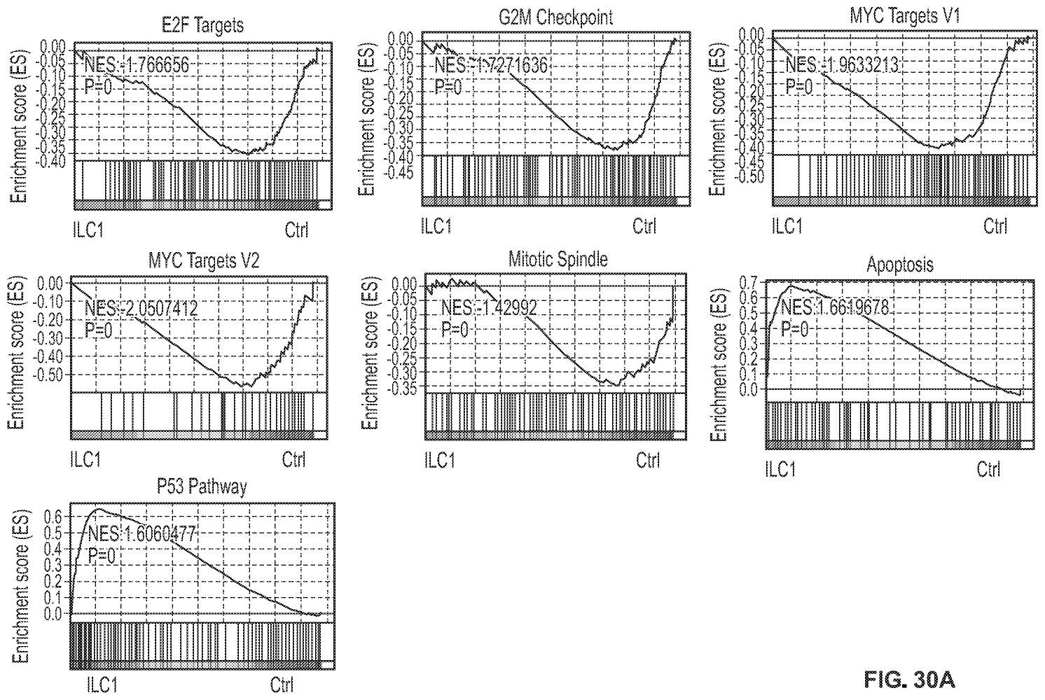

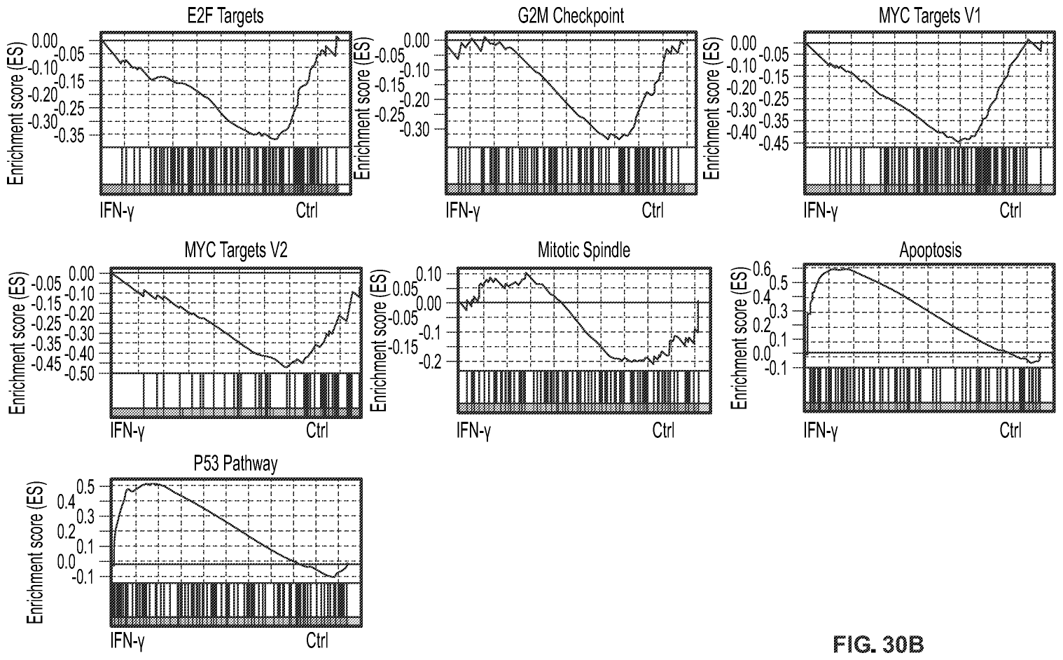

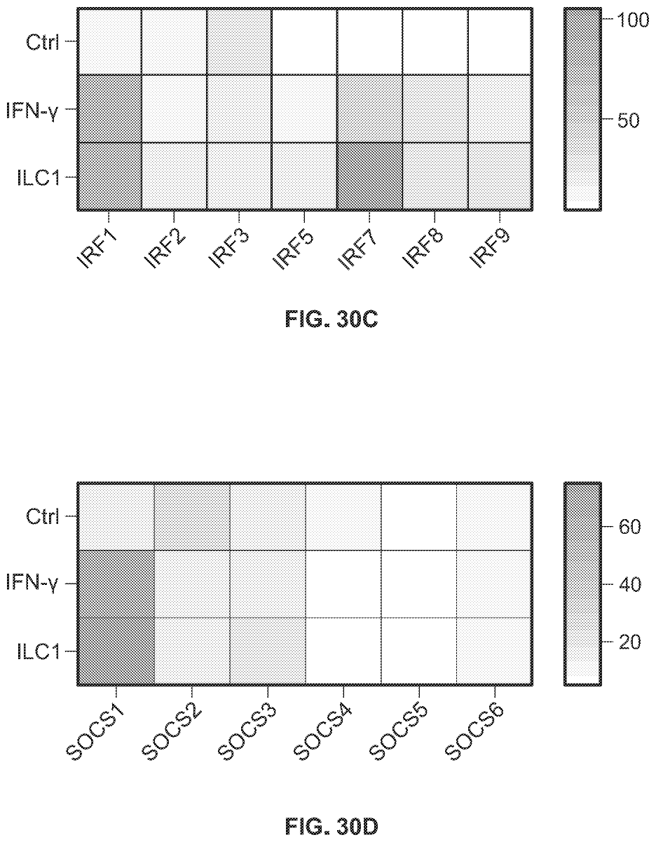

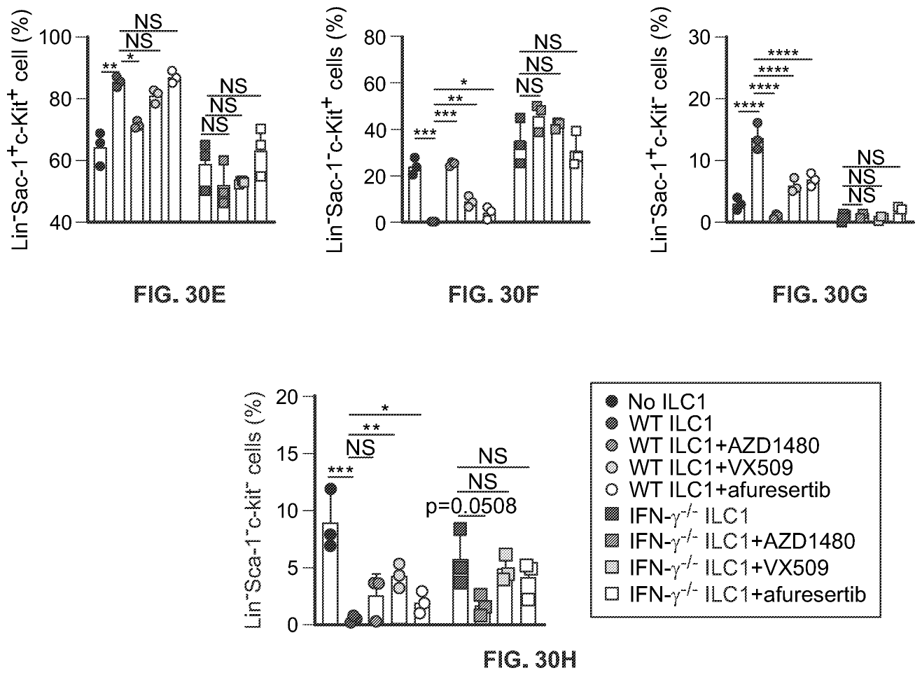

We conducted Ribozero RNA-seq analysis of LSCs co-cultured with or without ILC1s isolated from normal mice or mice treated with or without recombinant murine IFN-γ. Of note, after ILC1-LSC co-culture, we separated the LSCs from the ILC1s using FACS (FIG. 29A-29B). Subsequent RNA-seq revealed that, compared with untreated LSCs, the LSCs co-cultured with ILC1s had 445 significantly upregulated genes and 93 significantly downregulated genes. In LSCs co-cultured with recombinant IFN-γ, 320 genes were significantly upregulated and 82 were significantly downregulated (FIG. 29C). Furthermore, LSCs co-cultured with ILC1s or treated with IFN-γ had a large number of upregulated and downregulated genes in common (FIG. 19A and FIGS. 29D-29E), supporting our conclusion that ILC1s regulate LSCs by producing IFN-γ. Among the upregulated LSC genes unique to the ILC1-LSC co-culture, three of the top ten were chemokines (Ccl3, Ccl4, and Xcl1) (FIG. 29F). These data suggest that the interaction of ILC1s with LSCs may recruit additional immune cells into the tumor microenvironment (TME) to suppress the development of AML. Using gene set enrichment analysis (GSEA), we identified the top 10 pathways to which those upregulated and downregulated genes belonged (FIGS. 19B and 29G). LSCs co-cultured with ILC1s or treated with IFN-γ activated apoptotic pathways in LSCs while significantly suppressing E2F targets, G2M checkpoints, MYC targets, and mitotic spindle pathways. This is consistent with our finding that IFN-γ secreted by ILC1s increased apoptosis of LSCs (FIGS. 30A-30B). Additionally, after co-culture with ILC1s or treatment with IFN-γ, LSCs showed activation of the JAK-STAT and PI3K-AKT and signaling pathways (FIG. 19C) and increased expression of Akt3, Jak2, Stat1/2, Irf1/2/7/8/9, and suppressor of cytokine signaling 1 (Socs1), all of which are downstream of in the IFN-γ signaling pathway (FIGS. 19D-19F and FIGS. 30C-30D)³⁸⁻³⁹. This unbiased analysis further strengthened our conclusion that ILC1s regulate LSCs by secreting IFN-γ. It also suggests that ILC1s or their secreted IFN-γ utilize the JAK-STAT and PI3K-AKT signaling pathways to regulate LSCs. To confirm this, we pretreated LSCs for 30 min with inhibitors of signaling components involved in these two pathways; then we co-cultured them with WT ILC or IFN-γ^{-/-} ILC1s. Compared to co-culture with ILC alone, the JAK2 inhibitor AZD1480 or the JAK1/2/3 inhibitor VX-509 combined with ILC1s significantly increased LSCs differentiation into LS⁻K⁺ leukemic progenitor cells and decreased their differentiation into non-leukemic LS⁺K⁻ cells. There was no significant alteration in Lin⁻Sca-1⁻c-Kit⁻cell population (FIGS. 19G-19F and FIGS. 30E-30H). These findings suggest that ILC1s' effect on LSC differentiation is inhibited by blocking JAK-STAT and PI3K-AKT signaling. Predictably, the two JAK inhibitors did not affect LSCs co-cultured with IFN-γ^{-/-} ILC1s. We obtained similar results using afuresertib, an AKT inhibitor (FIGS. 19G-19H and FIGS. 30E-30H).

Collectively, these data suggest that IFN-γ derived from ILC1s regulates the differentiation of LSCs through JAK-STAT and PI3K-AKT signaling pathways.

### Example 7: ILC1s are Rapidly and Reproducibly Expanded and Exhibit Good Persistence

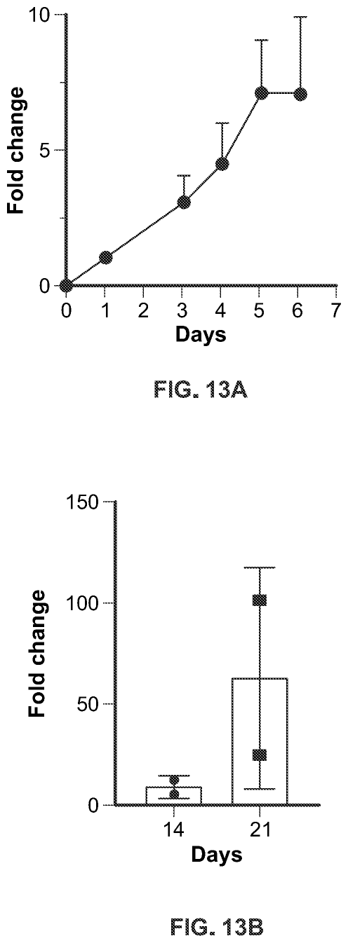

In FIG. 13A, 5,000-10,000 ILC1s isolated from mice liver were cultured with 1000U/ml human IL-2 and 10 ng/ml mouse IL-7 for 6 days. The fold change of ILC1s were shown (n=3).

In FIG. 13B, 10,000-40,000 ILC1s isolated from human PBMC were cultured with 1000U/ml human IL-2, 10 ng/ml human IL-7, and feeder cells K562 (the ratio of K562 and ILC1 is 1:1). The fold change of ILC1s were shown (n=2).

Ex vivo expanded ILC1 cells isolated using methods described herein exhibit rapid, reproducible expansion and show good persistence (FIGS. 13A-13B, FIGS. 4B-4D).

### Example 8: ILC1s—But Not NK Cells—Require DNAM-1 and IL-7Rα for IFN-γ Production When They Interact with LSCs

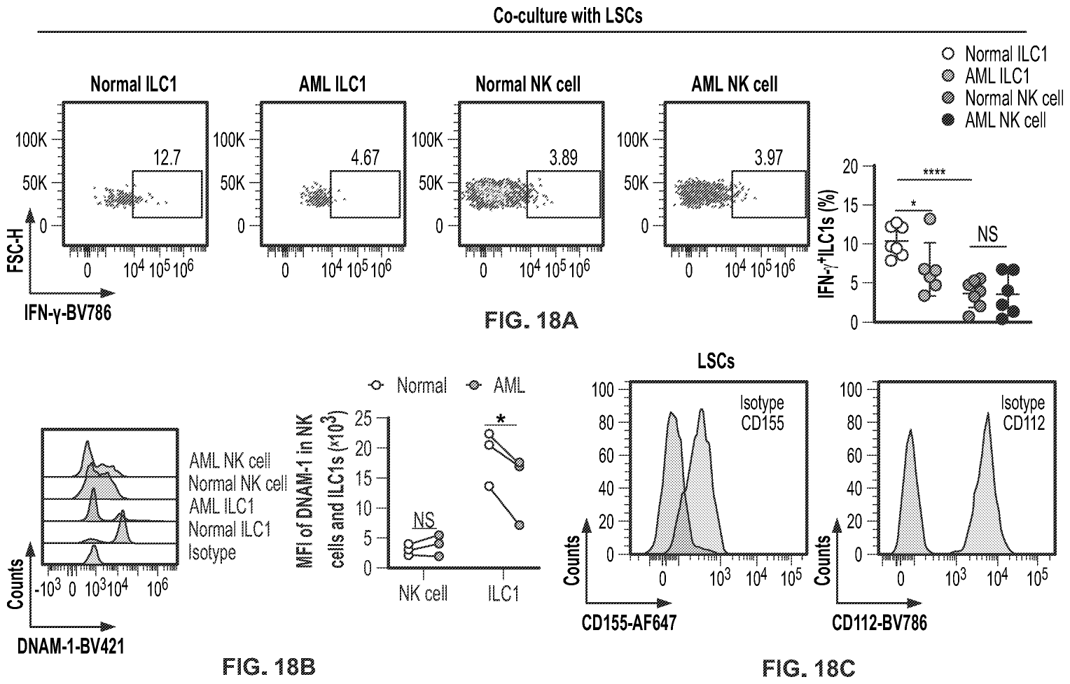

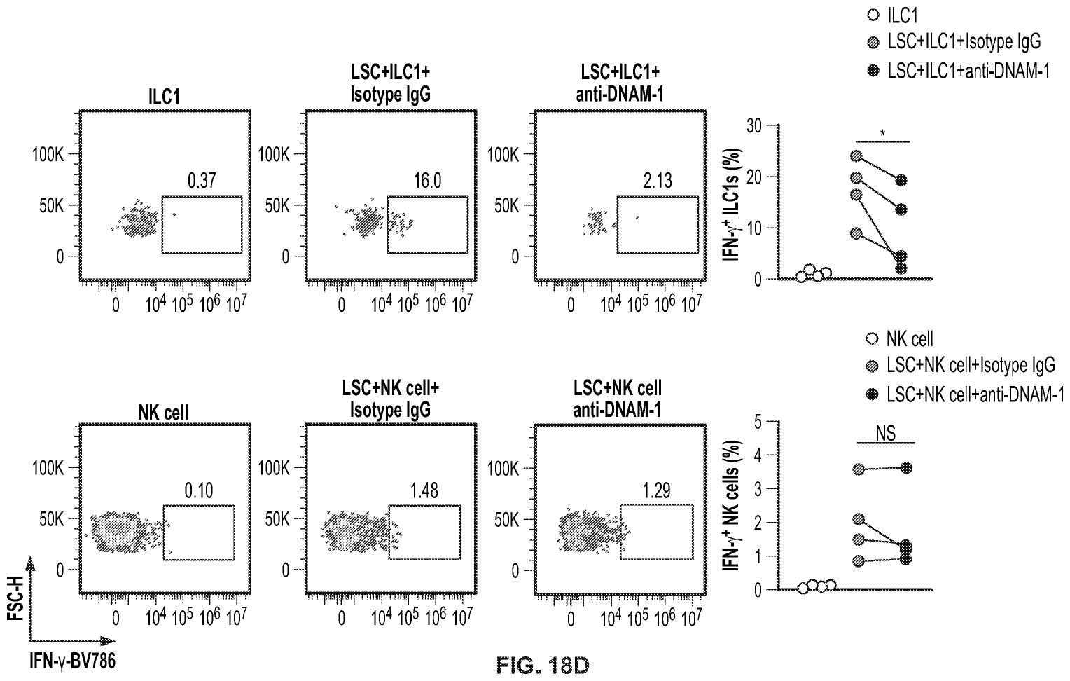

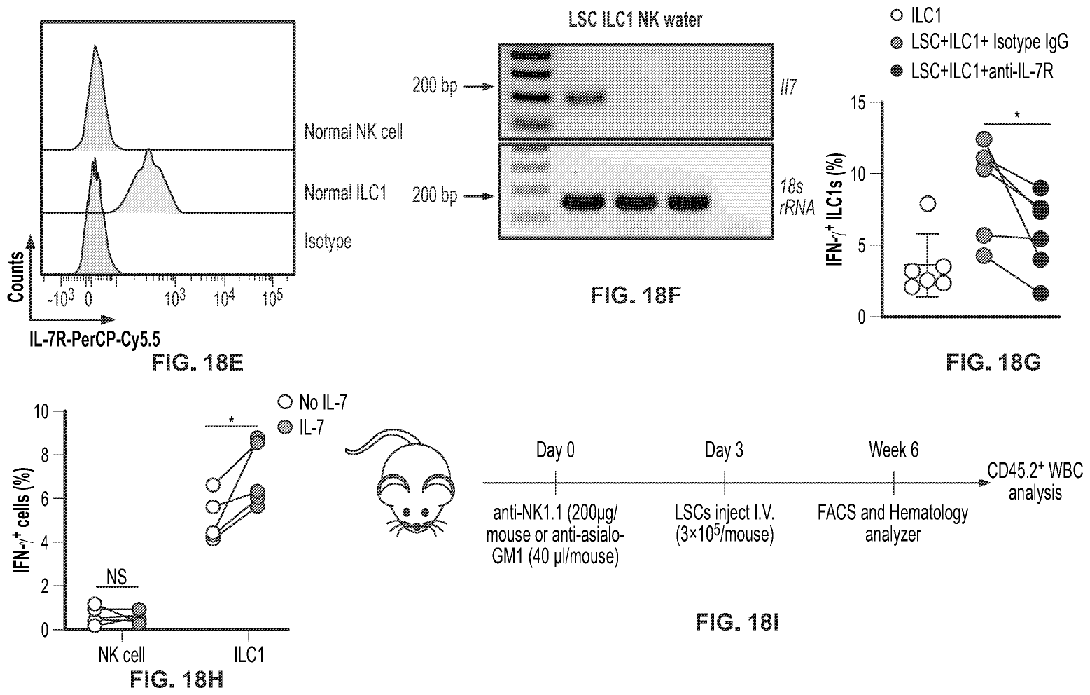

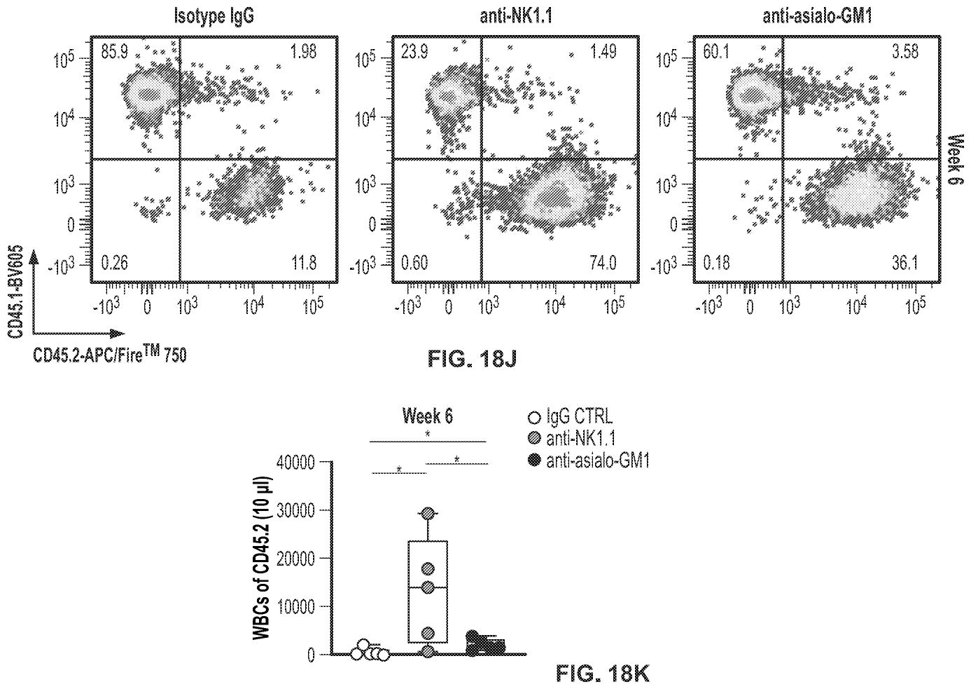

Both ILC1s and NK cells express IFN-γ, and we assessed each for their ability to produce IFN-γ in the presence or absence of AML or LSCs. We sorted those two cell types from the liver of normal mice and Mll^{PTD/WT}: Flt3^{ITD/ITD} mice with AML and co-cultured each preparation separately with LSCs. The ILC1s isolated from mice with AML produced significantly less IFN-γ than those from normal mice. This difference was not observed with the NK cells (FIG. 18A). Additionally, normal ILC1s co-cultured with LSCs produced more IFN-γ than the co-cultured NK cells (FIG. 18A). These results suggest that AML impairs IFN-γ production by liver ILC1s but not by liver NK cells, that normal liver ILC produce more IFN-γ than normal liver NK cells when they interact with LSCs, and ILC1-derived IFN-γ may play a more critical role than NK cells against LSCs.

Our data showed that ILC1s likely utilize cell—cell contact with LSCs to produce IFN-γ (FIG. 14O). This led us to conduct experiments to identify receptors and ligands needed for the effector and target cells to interact. The activation receptor DNAM-1, also known as CD226, is expressed more highly on ILC1s than on NK cells and is critical for IFN-γ production^{51,52}. To confirm that the receptor is also differentially expressed on the two types of innate immune cells (ILC1s and NK cells), we showed that DNAM-1 expression on AML ILC1s was significantly downregulated compared to DNAM-1 expression on normal ILC1s (FIG. 18B). In contrast, this downregulation was not observed on NK cells (FIG. 18B). We also discovered that both ligands of DNAM-1—CD155 and CD112—were highly expressed on LSCs (FIG. 18C). Thus, we hypothesized that ILC1s recognize LSCs at least partially through DNAM-1. As expected, DNAM-1 neutralizing antibody significantly blocked the production of IFN-γ in normal ILC but not in normal NK cells (compared to the control without DNAM-1 neutralizing antibody) (FIG. 18D). That blockade was only partial, however, so we searched for an additional mechanism underlying the interaction of ILC1s with LSCs. We focused on IL-7 receptor a (IL-7Rα), which is expressed during the development and maturation of all ILC subsets, including ILC1s, but is not expressed on liver NK cells⁶⁵⁻⁶⁶. Likewise, IL-7 plays an important role in the development of ILC but not NK cells^{67,68}. In line with the previous reports, we observed high expression of IL-7R on liver ILC1s but not on liver NK cells (FIG. 18E). We also discovered that LSCs produce IL-7 (FIG. 5f). Therefore, we suspected that the IL-7—IL-7R signaling pathway upregulates IFN-γ in normal ILC1s that are co-cultured with LSCs, as recently described in a non-cancer, liver-injury model¹⁸. Indeed, blockading IL-7-IL-7R signaling with an IL-7R neutralizing antibody downregulated the production of IFN-γ in normal ILC1s—but not in NK cells—after interaction with LSCs (FIGS. 18G and 28A). On the other hand, treatment with IL-7 significantly increased IFN-γ production in normal ILC1s but not in normal NK cells (FIGS. 18H and 28B). These results indicate that in the presence of LSCs, ILC1s are more potent IFN-γ producers than NK cells and this effect occurs at least in part via the interaction of ILC1 DNAM-1 and IL-7Rα with their cognate ligands expressed on LSCs, and thus ILC1s are becoming more effective suppressors of leukemia cells.

### Example 9: The Dominant Role of ILC1s and Their Collaboration with NK Cells in Controlling LSC

The above results do not suggest that liver NK cells are impotent against LSCs, as they did enhance apoptosis to some extent when the two cell types were co-cultured. However, IFN-γ neutralizing antibody did not affect their action, suggesting that, unlike ILC1s, the induction of LSC apoptosis by liver NK cells is not occurring primarily through IFN-γ (FIGS. 28C-28F).

To evaluate whether NK cells would slow the progression of AML in vivo, we i.p. injected anti-NK1.1 antibody (resulting in depletion of both NK cells and ILC1s) alone)⁶³ into immunocompetent recipient CD45.1 mice (FIGS. 18I and 28G). Three days later, we isolated LSCs from the spleen of CD45.2 Mll^{PTD/WT}: Flt3^{ITD/ITD} mice with AML and, i.v. injected those donor cells into the mouse depletion model. The preferential depletion of NK cells resulted in a small but significant increase in LSC-derived WBC counts when compared to non-depletion, while depletion of both ILC1s and NK cells produced a ˜20-fold increase in WBC cells when compared to an IgG control. The increase was >6-fold when it was compared to the anti-asialo GM1 antibody group with NK depletion alone (FIGS. 18J-18K). These data highlight the unique features of ILC1s and emphasize their collaboration with NK cells to control LSCs in leukemogenesis.

### Example 10: ILC1s Become Less Able to Target LSCs in AML

The data showed that ILC isolated from the liver of mice with AML produce less IFN-γ and TNF-α than ILC1s isolated from normal mice (FIG. 21D). To determine whether ILC in AML are less able to target LSCs, we sorted ILC from the liver of normal and Mll^{PTD/WT}: Flt3^{ITD/ITD} mice with AML, and co-cultured each of them with splenic LSCs isolated from the Mll^{PTD/WT}: Flt3^{ITD/ITD} mice with AML for 3 days. ILC1s isolated from mice with AML were less able to lyse LSCs compared to ILC1s from normal mice (FIG. 20A), resulting in increased LSC viability (FIG. 20B). We obtained similar results when we compared ILC1s isolated from patients with AML with those from healthy donors (FIGS. 20C-20D). Thus, ILC1s co-cultured with LSCs produced a significantly higher level of IFN-γ if they came from healthy donors rather than from patients with AML (FIG. 20E). Compared to normal ILC1s, those isolated from mice with AML also induced less LSC differentiation into Lin⁻Sca-1⁺c-Kit⁻ non-leukemic cell and were less able to suppress LSC differentiation into Lin⁻Sca-1⁻c-Kit⁺ leukemia progenitor cells (FIG. 20F-20H). However, when the IFN-γ neutralizing antibody was added to the culture, normal ILC1s and ILC1s isolated from mice with AML were similarly ineffective with LSCs (FIG. 20F-20H). These data suggest that the anti-leukemic function of ILC1s in humans with AML is as impaired as it is in mice.

### Example 11: Discussion

ILC1s play critical roles in inflammation and the early anti-viral response^{40,41,62}. However, their role in preventing and/or promoting cancer, including AML, has not been explored⁴². In particular, it is largely unknown whether ILC1s suppress or promote cancer development. Using in vitro studies in mouse and human as well as in vivo mouse models, we showed that the progression of AML can be controlled by normal ILC1s interacting with LSCs. We discovered that ILC1s have dual roles in regulating LSCs in AML: 1) ILC1s induce apoptosis of LSCs at high effector to target ratios; 2) At a lower dose of effector cells, ILC1s suppress the differentiation of LSCs into leukemia progenitor cells and then to myeloid blasts while facilitating the differentiation of LSCs into non-leukemic cells. Importantly, ILC1s do not affect the apoptosis and differentiation of normal stem cells. Without being bound by theory, although both IFN-γ and TNF-α are secreted by ILC1s, our work demonstrates that IFN-γ mediates ILC1-induced effects on LSCs via both the JAK-STAT and PI3K-AKT signaling pathways in mice. In addition, ILC1s produce higher levels of IFN-γ to control LSCs than do NK cells; DNAM-1 and IL-7Rα expressed on ILC1s interact with their cognate ligands expressed on LSCs. Thus, ILC1s may normally perform critical surveillance by spotting and destroying LSCs as well as other cancer stem cells; consequently, a dysfunction in this innate immune cell population can facilitate tumorigenesis and administering these cells can suppress tumorigenesis.

In AML patients who relapse, a small population of leukemia-initiating cells or LSCs is resistant to standard chemotherapy^{4,43}. Therefore, elucidating the mechanism(s) of LSC resistance is a critical unmet challenge, and developing novel approaches to targeting LSCs offers a potential strategy for prolonging relapse-free survival of patients with AML. Chemotherapy and targeted therapy (e.g., tyrosine kinase inhibitors including FDA-approved midostaurin and gilteritinib) can kill leukemic blasts but may also enrich LSCs^{44,45}. We show that normal ILC act directly on LSCs to control the progression of AML in vivo. Therefore, given the special biologic function of ILC1s, expanding autologous or normal allogeneic ILC ex vivo during times of remission or combining expanded ILC1s with an FDA-approved drug or cytokine may have a positive impact on prolonging relapse-free survival of patients with AML.

IFN-γ plays important roles in anti-viral and anti-tumor immunity, and has been used clinically to treat several diseases⁴⁶. However, IFN-γ-based therapies have at least two limitations that preclude routine clinical use for cancer patients. The first is that IFN-γ cannot be delivered into local tumor sites to subsequently achieve effective concentrations in the tumor microenvironment (TME) without significant toxicity⁴⁹⁻⁵¹. The second is that IFN-γ is rapidly cleared from the blood after intravenous administration, further limiting the ability to achieve effective local concentrations. These clinical disadvantages necessitate the development of alternative methods to ensure the effectiveness of IFN-γ in the local milieu of the marrow and/or other organs while limiting toxicity.

This application is the first to provide a promising new approach to treating AML: using a cell-based source of IFN-γ to target LSCs. Although ILC1 s are a minute cell population, they express abundant IFN-γ, especially when they interact with tumor cells in the TME. ILC1s also express high levels of chemokine receptors, including CXCR3 and CXCR6, the respective receptors for CXCL9-1 1 and CXCL16 that are expressed by AML cells^{41,50}. These receptor-ligand interactions may help recruit ILC1 s to the bone marrow or tumor sites, where most LSCs reside⁵¹.

Furthermore, ILC 1 s rapidly and persistently produce IFN-γ locally after contacting LSCs or more mature tumor cells, yielding sufficient cytokine to locally target AML blasts⁵³. Our data suggest that ILC1 s can also induce apoptosis and differentiation of LSCs within the TME. Moreover, ILC1 s are associated with reducing severe progression of graft-versus-host disease (GVHD) after allogeneic HSCT treatment for AML⁶⁵. This suggests that ILC1 s can control AML through their multifaceted roles.

Like ILC1s, NK cells also belong to group 1 ILCs⁶. Although more than a dozen studies have assessed the efficacy of infusing NK cells into patients in remission following AML treatment, some of which showed promising result⁵⁴, none have yet explored therapeutic ex vivo expansion and infusion of ILC1s during AML remission. Our data, especially our in vivo data, provide a strong rationale for developing methodologies to expand normal ILC1 populations rapidly and reproducibly for application as a cellular therapy to prolong relapse-free survival in patients with AML who achieve complete remission but may carry quiescent LSCs. This would be especially valuable for patients who are ineligible for HSCT.

The IFN-γ signaling pathway is associated with several biological responses and plays an important role in innate and adaptive immunity. It not only induces apoptosis of tumor cells⁵¹, but it also activates immune cells, two processes that are crucial for combatting cancer^{46,55}. IFN-γ induces PD-L1 expression in tumor cells, including AML blast cells⁵⁶ and immune cells⁵⁷⁻⁵⁸; it regulates PD-L1 expression mainly through the JAK1/2-STAT1/3-IRF1 axis in melanoma cells⁵⁹. Our data demonstrates that both ILC1 s and recombinant IFN-γ block the differentiation of LSCs into leukemia progenitor cells through the JAK-STAT signaling pathway. This suggests that IFN-γ has a broad reach, covering both tumor cells and immune cells, as well as both mature tumor cells and cancer-stem-like cells among which it can induce different outcomes. The action of IFN-γ on tumors, tumor stem cells, and immune cells can induce PD-L1 expression, which can block T cell responses to tumor cells and their stem cell⁶⁰ , differentiation of cancer stem cells, and activation of immune cells⁶¹. Although these roles are complex and clinical use of IFN-γ should consider all of these effects, the ability of an anti-PD-L1 antibody to block the adverse effects of IFN-γ-upregulated PD-L1 provides a good rationale for combining IFN-γ or if too toxic, combining cells that produce this cytokine, such as ILC1s, with anti-PD-L1 antibody to treat cancers, including AML. Such an anti-leukemic approach may bring new hope to patients with AML, especially relapsed older patients who have a dismal prognosis.

In summary, this study identified novel functions of ILC1s: they can closely regulate AML LSCs by inducing apoptosis; they prevent LSCs from differentiating into leukemia progenitors and then myeloid blasts; and they promote the differentiation of LSCs into a non-leukemic lineage. All of these actions are mediated by IFN-γ that ILC1s secrete when they form cell—cell contact with LSCs. We therefore believe that, by uncovering the mechanisms underlying these processes, our study could unveil a new immunotherapeutic approach—administration of ILC1s that have been expanded ex vivo—to prolong relapse-free survival of patients diagnosed with AML.

### REFERENCES

1 Nair, R., Salinas-Illarena, A. & Baldauf, H. M. New strategies to treat AML: novel insights into AML survival pathways and combination therapies. Leukemia, doi:10.1038/s41375-020-01069-1 (2020).

2 Ballester, G., Tirona, M. T. & Ballester, O. Hematopoietic stem cell transplantation in the elderly. Oncology (Williston Park, N. Y.) 21, 1576-1583; discussion 1587, 1590-1571, 1606 (2007).

3 Siveen, K. S., Uddin, S. & Mohammad, R. M. Targeting acute myeloid leukemia stem cell signaling by natural products. Molecular cancer 16, 13, doi:10.1186/s12943-016-0571-x (2017).

4 Yamashita, M., Dellorusso, P. V., Olson, O. C. & Passegué, E. Dysregulated haematopoietic stem cell behaviour in myeloid leukaemogenesis. Nature reviews. Cancer 20, 365-382, doi:10.1038/s41568-020-0260-3 (2020).

5 Shlush, L. I. et al. Identification of pre-leukaemic haematopoietic stem cells in acute leukaemia. Nature 506, 328-333, doi:10.1038/nature13038 (2014).

6 Klose, C. S. N. & Artis, D. Innate lymphoid cells control signaling circuits to regulate tissue-specific immunity. Cell research 30, 475-491, doi:10.1038/s41422-020-0323-8 (2020).

7 Colonna, M. Innate Lymphoid Cells: Diversity, Plasticity, and Unique Functions in Immunity. Immunity 48, 1104-1117, doi:10.1016/j.immuni.2018.05.013 (2018).

8 Diefenbach, A., Colonna, M. & Koyasu, S. Development, differentiation, and diversity of innate lymphoid cells. Immunity 41, 354-365, doi:10.1016/j.immuni.2014.09.005 (2014).

9 Moral, J. A. et al. ILC2s amplify PD-1 blockade by activating tissue-specific cancer immunity. Nature 579, 130-135, doi:10.1038/s41586-020-2015-4 (2020).

Chevalier, M. F. et al. ILC2-modulated T cell-to-MDSC balance is associated with bladder cancer recurrence. The Journal of clinical investigation 127, 2916-2929, doi:10.1172/jci89717 (2017).

11 Koh, J. et al. IL23-Producing Human Lung Cancer Cells Promote Tumor Growth via Conversion of Innate Lymphoid Cell 1 (ILC1) into ILC3. Clinical cancer research: an official journal of the American Association for Cancer Research 25, 4026-4037, doi:10.1158/1078-0432.Ccr-18-3458 (2019).

12 Carrega, P. et al. NCR(+)ILC3 concentrate in human lung cancer and associate with intratumoral lymphoid structures. Nature communications 6, 8280, doi:10.1038/ncomms9280 (2015).

13 Zhang, Y. et al. IL-22 promotes tumor growth of breast cancer cells in mice. Aging 12, 13354-13364, doi:10.18632/aging.103439 (2020).

14 Eisenring, M., vom Berg, J., Kristiansen, G., Saller, E. & Becher, B. IL-12 initiates tumor rejection via lymphoid tissue-inducer cells bearing the natural cytotoxicity receptor NKp46. Nature immunology 11, 1030-1038, doi:10.1038/ni.1947 (2010).

15 Jovanovic, I. P. et al. Interleukin-33/ST2 axis promotes breast cancer growth and metastases by facilitating intratumoral accumulation of immunosuppressive and innate lymphoid cells. International journal of cancer 134, 1669-1682, doi:10.1002/ijc.28481 (2014).

16 Seillet, C., Belz, G. T. & Huntington, N. D. Development, Homeostasis, and Heterogeneity of NK Cells and ILC1. Current topics in microbiology and immunology 395, 37-61, doi:10.1007/82_2015_474 (2016).

17 Wang, Y., Dong, W., Zhang, Y., Caligiuri, M. A. & Yu, J. Dependence of innate lymphoid cell 1 development on NKp46. PLoS biology 16, e2004867, doi:10.1371/journal.pbio.2004867 (2018).

18 Mopin, A., Driss, V. & Brinster, C. A Detailed Protocol for Characterizing the Murine C1498 Cell Line and its Associated Leukemia Mouse Model. Journal of visualized experiments: JoVE, doi:10.3791/54270 (2016).

19 Almishri, W. et al. TNFα Augments Cytokine-Induced NK Cell IFNγ Production through TNFR2. Journal of innate immunity 8, 617-629, doi:10.1159/000448077 (2016).

20 Zhang, B. et al. Altered microenvironmental regulation of leukemic and normal stem cells in chronic myelogenous leukemia. Cancer cell 21, 577-592, doi:10.1016/j.ccr.2012.02.018 (2012).

21 Zhang, B. et al. Bone marrow niche trafficking of miR-126 controls the self-renewal of leukemia stem cells in chronic myelogenous leukemia. Nature medicine 24, 450-462, doi:10.1038/nm.4499 (2018).

22 Zorko, N. A. et al. Mll partial tandem duplication and Flt3 internal tandem duplication in a double knock-in mouse recapitulates features of counterpart human acute myeloid leukemias. Blood 120, 1130-1136, doi:10.1182/blood-2012-03-415067 (2012).

23 Artis, D. & Spits, H. The biology of innate lymphoid cells. Nature 517, 293-301, doi:10.1038/nature14189 (2015).

24 Appelbaum, F. R., Rowe, J. M., Radich, J. & Dick, J. E. Acute myeloid leukemia. Hematology. American Society of Hematology. Education Program, 62-86, doi:10.1182/asheducation-2001.1.62 (2001).

25 Hoshii, T. et al. mTORC1 is essential for leukemia propagation but not stem cell self-renewal. The Journal of clinical investigation 122, 2114-2129, doi:10.1172/jci62279 (2012).

26 Kumar, R., Fossati, V., Israel, M. & Snoeck, H. W. Lin-Scal+kit- bone marrow cells contain early lymphoid-committed precursors that are distinct from common lymphoid progenitors. Journal of immunology (Baltimore, Md.: 1950) 181, 7507-7513, doi:10.4049/jimmunol.181.11.7507 (2008).

27 Peng, C. et al. LSK derived LSK− cells have a high apoptotic rate related to survival regulation of hematopoietic and leukemic stem cells. PLoS One 7, e38614, doi:10.1371/journal.pone.0038614 (2012).

28 Thomas, D. & Majeti, R. Biology and relevance of human acute myeloid leukemia stem cells. Blood 129, 1577-1585, doi:10.1182/blood-2016-10-696054 (2017).

29 Joshi, K., Zhang, L., Breslin, S. J. P. & Zhang, J. Leukemia Stem Cells in the Pathogenesis, Progression, and Treatment of Acute Myeloid Leukemia. Advances in experimental medicine and biology 1143, 95-128, doi:10.1007/978-981-13-7342-8 5 (2019).

30 Graf, M. et al. Expression of MAC-1 (CD11b) in acute myeloid leukemia (AML) is associated with an unfavorable prognosis. American journal of hematology 81, 227-235, doi:10.1002/ajh.20526 (2006).

31 Adane, B. et al. The Hematopoietic Oxidase NOX2 Regulates Self-Renewal of Leukemic Stem Cells. Cell reports 27, 238-254.e236, doi:10.1016/j.celrep.2019.03.009 (2019).

32 Park, S. M. et al. IKZF2 Drives Leukemia Stem Cell Self-Renewal and Inhibits Myeloid Differentiation. Cell stem cell 24, 153-165.e157, doi:10.1016/j.stem.2018.10.016 (2019).

33 Sharma, A. et al. Constitutive IRF8 expression inhibits AML by activation of repressed immune response signaling. Leukemia 29, 157-168, doi:10.1038/1eu.2014.162 (2015).

34 Fehniger, T. A. et al. Fatal leukemia in interleukin 15 transgenic mice follows early expansions in natural killer and memory phenotype CD8+ T cells. The Journal of experimental medicine 193, 219-231, doi:10.1084/jem.193.2.219 (2001).

35 Bank, U. et al. c-FLIP is crucial for IL-7/IL-15-dependent NKp46(+) ILC development and protection from intestinal inflammation in mice. Nature communications 11, 1056, doi:10.1038/s41467-020-14782-3 (2020).

36 Klose, C. S. & Diefenbach, A. Transcription factors controlling innate lymphoid cell fate decisions. Current topics in microbiology and immunology 381, 215-255, doi:10.1007/82_2014_381 (2014).

37 Pandey, R. et al. SHP2 inhibition reduces leukemogenesis in models of combined genetic and epigenetic mutations. The Journal of clinical investigation 129, 5468-5473, doi:10.1172/jci130520 (2019).

38 Villarino, A. V., Kanno, Y. & O'Shea, J. J. Mechanisms and consequences of Jak-STAT signaling in the immune system. Nature immunology 18, 374-384, doi:10.1038/ni.3691 (2017).

39 Shuai, K. & Liu, B. Regulation of JAK—STAT signalling in the immune system. Nature Reviews Immunology 3, 900-911, doi:10.1038/nri1226 (2003).

40 Wang, S. et al. Transdifferentiation of tumor infiltrating innate lymphoid cells during progression of colorectal cancer. Cell research 30, 610-622, doi:10.1038/s41422-020-0312-γ (2020).

41 Weizman, O. E. et al. ILC1 Confer Early Host Protection at Initial Sites of Viral Infection. Cell 171, 795-808.e712, doi:10.1016/j.ce11.2017.09.052 (2017).

42 Crinier, A. et al. Multidimensional molecular controls defining NK/ILC1 identity in cancers. Seminars in immunology, 101424, doi:10.1016/j.smim.2020.101424 (2020).

43 Taussig, D. C. et al. Leukemia-initiating cells from some acute myeloid leukemia patients with mutated nucleophosmin reside in the CD34(−) fraction. Blood 115, 1976-1984, doi:10.1182/blood-2009-02-206565 (2010).

44 Trabanelli, S. et al. CD127+ innate lymphoid cells are dysregulated in treatment naïve acute myeloid leukemia patients at diagnosis. Haematologica 100, e257-260, doi:10.3324/haematol.2014.119602 (2015).

45 Levis, M. & Perl, A. E. Gilteritinib: potent targeting of FLT3 mutations in AML. Blood advances 4, 1178-1191, doi:10.1182/bloodadvances.2019000174 (2020).

46 Castro, F., Cardoso, A. P., Gonçalves, R. M., Serre, K. & Oliveira, M. J. Interferon-Gamma at the Crossroads of Tumor Immune Surveillance or Evasion. Frontiers in immunology 9, 847, doi:10.3389/fimmu.2018.00847 (2018).

47 Sharma, A. & Jusko, W. J. Characteristics of indirect pharmacodynamic models and applications to clinical drug responses. British journal of clinical pharmacology 45, 229-239, doi:10.1046/j.1365-2125.1998.00676.x (1998).

48 Razaghi, A., Owens, L. & Heimann, K. Review of the recombinant human interferon gamma as an immunotherapeutic: Impacts of production platforms and glycosylation. Journal of biotechnology 240, 48-60, doi:10.1016/j.jbiotec.2016.10.022 (2016).

49 Kurzrock, R. et al. Pharmacokinetics, single-dose tolerance, and biological activity of recombinant gamma-interferon in cancer patients. Cancer research 45, 2866-2872 (1985).

50 Maloy, K. J. & Uhlig, H. H. ILC1 populations join the border patrol. Immunity 38, 630-632, doi:10.1016/j.immuni.2013.03.005 (2013).

51 Houshmand, M. et al. Bone marrow microenvironment: The guardian of leukemia stem cells. World journal of stem cells 11, 476-490, doi:10.4252/wjsc.v11.i8.476 (2019).

52 Dunn, G. P., Koebel, C. M. & Schreiber, R. D. Interferons, immunity and cancer immunoediting. Nature reviews. Immunology 6, 836-848, doi:10.1038/nri1961 (2006).

53 Munneke, J. M. et al. Activated innate lymphoid cells are associated with a reduced susceptibility to graft-versus-host disease. Blood 124, 812-821, doi:10.1182/blood-2013-11-536888 (2014).

54 Berrien-Elliott, M. M. et al. Multidimensional Analyses of Donor Memory-Like NK Cells Reveal New Associations with Response after Adoptive Immunotherapy for Leukemia. Cancer discovery 10, 1854-1871, doi:10.1158/2159-8290.Cd-20-0312 (2020).

55 Gresser, I. Biologic effects of interferons. The Journal of investigative dermatology 95, 66s-71s, doi:10.1111/1523-1747.ep12874776 (1990).

56 Krönig, H. et al. Interferon-induced programmed death-ligand 1 (PD-L1/B7-H1) expression increases on human acute myeloid leukemia blast cells during treatment. European journal of haematology 92, 195-203, doi:10.1111/ejh.12228 (2014).

57 Hartley, G. et al. Immune regulation of canine tumour and macrophage PD-L1 expression. Veterinary and comparative oncology 15, 534-549, doi:10.1111/vco.12197 (2017).

58 Munir, S. et al. Inflammation induced PD-L1-specific T cells. Cell stress 3, 319-327, doi:10.15698/cst2019.10.201 (2019).

59 Garcia-Diaz, A. et al. Interferon Receptor Signaling Pathways Regulating PD-L1 and PD-L2 Expression. Cell reports 19, 1189-1201, doi:10.1016/j.celrep.2017.04.031 (2017).

60 Srivastava, S. et al. Immunogenic Chemotherapy Enhances Recruitment of CAR-T Cells to Lung Tumors and Improves Antitumor Efficacy when Combined with Checkpoint Blockade. Cancer cell, doi.org/10.1016/j.ccell.2020.11.005 (2020).

61 Song, M. et al. Low-Dose IFNγ Induces Tumor Cell Stemness in Tumor Microenvironment of Non-Small Cell Lung Cancer. Cancer research 79, 3737-3748, doi:10.1158/0008-5472.Can-19-0596 (2019).

62. Shannon, J. P. et al. Group 1 innate lymphoid-cell-derived interferon-γ maintains anti-viral vigilance in the mucosal epithelium. Immunity (2021).

63. Nabekura, T., Riggan, L., Hildreth, A. D., O'Sullivan, T. E. & Shibuya, A. Type 1 Innate Lymphoid Cells Protect Mice from Acute Liver Injury via Interferon-γ Secretion for Upregulating Bc1-xL Expression in Hepatocytes. Immunity 52, 96-108.e109 (2020).

64. Gao, Y. et al. Tumor immunoevasion by the conversion of effector NK cells into type 1 innate lymphoid cells. Nature immunology 18, 1004-1015 (2017).

65. Robinette, M. L. et al. Transcriptional programs define molecular characteristics of innate lymphoid cell classes and subsets. Nat Immunol 16, 306-317 (2015).

66. Sojka, D. K. et al. Tissue-resident natural killer (NK) cells are cell lineages distinct from thymic and conventional splenic NK cells. Elife 3, e01659 (2014).

67. Spits, H., Bernink, J. H. & Lanier, L. NK cells and type 1 innate lymphoid cells: partners in host defense. Nat Immunol 17, 758-764 (2016).

68. Klose, C. S. N. et al. Differentiation of type 1 ILCs from a common progenitor to all helper-like innate lymphoid cell lineages. Cell 157, 340-356 (2014).

69. Smith, M. et al. Adult acute myeloid leukaemia. Critical reviews in oncology/hematology 50, 197-222 (2004).

70. Filén, J. J. et al. Quantitative proteomics reveals GIMAP family proteins 1 and 4 to be differentially regulated during human T helper cell differentiation. Molecular & cellular proteomics : MCP 8, 32-44 (2009).

71. Laouedj, M. et al. S100A9 induces differentiation of acute myeloid leukemia cells through TLR4. Blood 129, 1980-1990 (2017).

72. Shipounova Nifontova, I. N., Bigil'diev, A. E., Svinareva, D. A. & Drize, N. I. Characteristics of leukemia stem cells of murine myeloproliferative disease involving the liver. Bulletin of experimental biology and medicine 149, 293-297 (2010).

74. Bolger, A. M., Lohse, M. & Usadel, B. Trimmomatic: a flexible trimmer for Illumina sequence data. Bioinformatics (Oxford, England) 30, 2114-2120 (2014).

15 75. Chen, S., Zhou, Y., Chen, Y. & Gu, J. fastp: an ultra-fast all-in-one FASTQ preprocessor. Bioinformatics (Oxford, England) 34, i884-i890 (2018).

76. Dobin, A. et al. STAR: ultrafast universal RNA-seq aligner. Bioinformatics (Oxford, England) 29, 15-21 (2013).

77. Anders, S. & Huber, W. Differential expression analysis for sequence count data. Genome biology 11, R106 (2010).

78. Robinson, M. D., McCarthy, D. J. & Smyth, G. K. edgeR: a Bioconductor package for differential expression analysis of digital gene expression data. Bioinformatics (Oxford, England) 26, 139-140 (2010).

79. Mootha, V. K. et al. PGC-lalpha-responsive genes involved in oxidative phosphorylation are coordinately downregulated in human diabetes. Nature genetics 34, 267-273 (2003).

80. Subramanian, A. et al. Gene set enrichment analysis: a knowledge-based approach for interpreting genome-wide expression profiles. Proceedings of the National Academy of Sciences of the United States of America 102, 15545-15550 (2005).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method comprising:
(c) isolating a population of type I innate lymphoid cells (ILC1s); and
(d) culturing the population of ILC1s in growth media under conditions and for a time to expand the population of ILC1s.

2. The method of claim 1, wherein the population of ILC1s are human.

3. The method of claim 2, wherein the population of ILC1s are isolated from blood, peripheral blood, or peripheral blood mononuclear cells (PBMCs).

4. The method of claim 2, wherein the population of ILC1s comprise 50%, 60%, 70%, 80%, 85%, 90%, 95%, 96%, 97%, 98%, 99%, or 100% ILC1s.

5. The method of claim 4, wherein at least 50%, at least 60%, at least 70%, at least 80%, at least 85%, at least 90%, at least 95%, at least 96%, at least 97%, at least 98%, or at least 99% of the cells in the population of ILC1s comprise cells selected from:
Lin⁻CD56⁻CD127⁺c-Kit⁻CRTH2⁻,
Lin⁻CD56⁻CD127⁺,
Lin⁻CD56⁻CD127⁺c-Kit⁻,
Lin⁻CD56⁻CD127⁺c-Kit⁻CRTH2⁻EOMES⁻,
Lin⁻CD56⁻CD127⁺c-Kit⁻CRTH2⁻CXCR3⁺CXCR6⁺, or
Lin⁻CD56⁻CD127⁺c-Kit⁻CRTH2⁻EOMES⁻CXCR3⁺CXCR6⁺.

6. The method of any one of the preceding claims, wherein the population of ILC1s is contacted with or cultured in the presence of at least one of IL-2, IL-12, IL-15, or IL-7.

7. The method of any one of the preceding claims, wherein the isolated population of ILC1s is co-cultured with feeder cells.

8. The method of claim 7, wherein the feeder cells comprise K562 cells.

9. The method of any one of claim 7 or 8, wherein the ILC1feeder cell ratio is 1:1, 1:2, 1:3, 1:4, 1:5, 2:1, 3:1, 4:1, or 5:1.

10. An isolated population of ILC1 cells, wherein at least 50%, at least 60%, at least 30 70%, at least 80%, at least 85%, at least 90%, at least 95%, at least 96%, at least 97%, at least 98%, or at least 99% of the cells are selected from:
Lin⁻CD56⁻CD127⁺c-Kit⁻CRTH2⁻,
Lin⁻CD56⁻CD127⁺,
Lin⁻CD56⁻CD127⁺c-Kit⁻,
35 Lin⁻CD56⁻CD127⁺c-Kit⁻CRTH2⁻EOMES⁻,
Lin⁻CD56⁻CD127⁺c-Kit⁻CRTH2⁻CXCR3⁺CXCR6⁺, or
Lin⁻CD56⁻CD127⁺c-Kit⁻CRTH2⁻EOMES⁻CXCR3⁺ CXCR6⁺.

11. A composition comprising the population of ISCls of any of the preceding claims.

12. A method of treating a cancer or leukemia, the method comprising administering to a subject in need thereof a population of ex vivo expanded ILC or the composition of claim 11.

13. A method of killing, eliminating, or reducing cancer cells, leukemia cells, leukemia stem cells (LSCs), leukemia progenitor cells, myeloid blasts, or cells expressing CXCL9-11 or CXCL16, the method comprising administering to a subject in need thereof a population of ex vivo expanded ILC or the composition of claim 11.

14. A method of reducing or ameliorating a symptom associated with a cancer or leukemia, the method comprising administering to a subject in need thereof a population of ex vivo expanded ILC or the composition of claim 11.

15. A method of inhibiting or reducing leukemogenesis, the method comprising administering to a subject in need thereof a population of ex vivo expanded ILC1s or the composition of claim 11.

16. A method of inhibiting or reducing differentiation of LSCs into leukemia progenitor cells or myeloid blasts, the method comprising administering to a subject in need thereof a population of ex vivo expanded ILC or the composition of claim 11.

17. A method of promoting or increasing differentiation of LSCs to non-leukemic cells, the method comprising administering to a subject in need thereof a population of ex vivo expanded ILC or the composition of claim 11.

18. A method of prolonging relapse-free survival, preventing relapse, or decreasing the risk of relapse in a cancer or leukemia patient, the method comprising administering to a subject in need thereof a population of ex vivo expanded ILC1s or the composition of claim 11.

19. A method of increasing INF-γ concentration or prolonging INF-γ presence in a tumor microenvironment, the method comprising administering to a subject in need thereof a population of ex vivo expanded ILC or the composition of claim 11.

20. The method of any of claims 12-19, wherein the ex vivo expanded ILC1s are human.

21. The method of any of claims 12-19, wherein the ILC1s of the composition or the ex vivo expanded ILC1s are autologous or allogenic.

22. The method of claim 21, wherein the autologous ILC1s are isolated from the patient during remission or any cancer-free time.

23. The method of any of claims 12-19, wherein the population of ex vivo expanded ILC1 cells or the composition is administered in single or repeat dosing.

24. The method of any of claims 12-23, wherein an effective amount of the population of ex vivo expanded ILC1 cells or the composition is administered.

25. The method of any of claims 12-24, wherein the population of ex vivo expanded ILC1 cells or the composition is administered locally or systemically.

26. The method of any of claims 12-25, wherein the population of ex vivo expanded ILC1s or the composition is infused or administered intravenously, locally or directly injected, injected into tumor microenvironment, or administered intratumorally.

27. The method of any of claims 12-26, wherein at least one symptom of a cancer or a leukemia is reduced, ameliorated, or relieved.

28. The method of any one of claims 12, 14, 16, 18, and 27, wherein the leukemia is any one of acute lymphocytic leukemia (ALL), acute myeloid leukemia (AML), chronic lymphocytic leukemia (CLL), chronic myeloid leukemia (CML), hairy cell leukemia (HCL), or myelodysplastic syndromes (MDS).

29. The method of any of claims 12-28, wherein the population of ex vivo expanded ILC1 cells or the composition is administered before remission, during remission, or during relapse.

30. The method of any of claims 12-29, wherein the population of ex vivo expanded ILC1 cells or the composition is administered before, after, or in combination with one or more of IFN-γ, a cytokine, IL-15, an anti-PD-L1 antibody or a PD-L1 inhibitor, an anti-PD-1 antibody or a PD-1 inhibitor, a chemotherapy, a kinase inhibitor (e.g., midostaurin and gilteritinib), or radiation therapy.

31. The isolated population of ILC1 cells of any of claim 10, wherein the cells comprise a vector or recombinant nucleic acid molecule encoding human IL-15 and/or human IL-12.
